SEQUENCE LISTING (30 sequences)

SEQ ID NO: 1 - DNA, 22 bp, artificial
  primer
cgcgtgaaggatgaaggttttc

SEQ ID NO: 2 - DNA, 20 bp, artificial
  primer
tcagttaaagcccagcaggc

SEQ ID NO: 3 - DNA, 21 bp, artificial
  primer
cgcggtaaaacgtaggtcaca

SEQ ID NO: 4 - DNA, 22 bp, artificial
  primer
ctgggacgttgtttctgagttt

SEQ ID NO: 5 - DNA, 22 bp, artificial
  primer
ggaatcttccacaatggacgca

SEQ ID NO: 6 - DNA, 22 bp, artificial
  primer
catggagttccactgtcctctt

SEQ ID NO: 7 - DNA, 22 bp, artificial
  primer
gtccatcgcttaacggtggatc

SEQ ID NO: 8 - DNA, 17 bp, artificial
  primer
accacctgtgaacccgc

SEQ ID NO: 9 - DNA, 18 bp, artificial
  primer
gcactccacctggggagt

SEQ ID NO: 10 - DNA, 22 bp, artificial
  primer
caaccttcctccgggttatcca

SEQ ID NO: 11 - DNA, 20 bp, artificial
  primer
ggcttgctggacaaatactg

SEQ ID NO: 12 - DNA, 20 bp, artificial
  primer
ctaggctcgtcagaaggatg

SEQ ID NO: 13 - DNA, 18 bp, artificial
  primer
ggtaggggagggtggaac

SEQ ID NO: 14 - DNA, 17 bp, artificial
  primer
gcggtcccgcgtgggtt

SEQ ID NO: 15 - DNA, 22 bp, artificial
  primer
ggagtgctaataccggatgtga

SEQ ID NO: 16 - DNA, 20 bp, artificial
  primer
ccgtggcttcctcgtttact

SEQ ID NO: 17 - DNA, 24 bp, artificial
  primer
cgtgaaggaagaagtatctcggta

SEQ ID NO: 18 - DNA, 21 bp, artificial
  primer
tcagttaccgtccagcaggcc

SEQ ID NO: 19 - DNA, 20 bp, artificial
  primer
  misc_feature (4)..(4): s is g or c
  misc_feature (9)..(9): y is t or c
  misc_feature (12)..(12): y is t or c
gtgstgcayggytgtcgtca

SEQ ID NO: 20 - DNA, 20 bp, artificial
  primer
  misc_feature (6)..(6): r is g or a
  misc_feature (12)..(12): r is g or a
acgtcrtccccrccttcctc

SEQ ID NO: 21 - DNA, 17 bp, artificial
  primer
attaccgcggctgctgg

SEQ ID NO: 22 - DNA, 15 bp, artificial
  primer
  misc_feature (14)..(14): r is g or a
acgggcggtgtgtrc

SEQ ID NO: 23 - DNA, 21 bp, artificial
  primer
  misc_feature (9)..(9): n is a, c, g, or t
cgggtgctncccactttcatg

SEQ ID NO: 24 - DNA, 18 bp, artificial
  primer
  misc_feature (2)..(2): n is a, c, g, or t
gntaccttgttacgactt

SEQ ID NO: 25 - DNA, 22 bp, artificial
  primer
  misc_feature (6)..(6): y is t or c
tacggytaccttgttacgactt

SEQ ID NO: 26 - DNA, 17 bp, artificial
  primer
  misc_feature (10)..(10): w is a or t
  misc_feature (15)..(15): r is g or a
aaggaggtgwtccarcc

SEQ ID NO: 27 - DNA, 18 bp, artificial
  primer
  misc_feature (12)..(12): m is a or c
agagtttgatcmtggctc

SEQ ID NO: 28 - DNA, 21 bp, artificial
  primer
gattctggctcaggatgaacg

SEQ ID NO: 29 - DNA, 20 bp, artificial
  primer
  misc_feature (12)..(12): m is a or c
agagtttgatcmtggctcag

SEQ ID NO: 30 - DNA, 20 bp, artificial
  primer
ccagcagccgcggtaatacg

(19) United States
(12) Patent Application Publication
(10) Pub. No.: US 2021/0355431 A1
(43) Pub. Date: Nov. 18, 2021

(54) METHOD OF MANUFACTURING A CONSORTIUM OF BACTERIAL STRAINS

(71) Applicant: PHARMABIOME AG, SCHLIEREN (CH)

(72) Inventors: FABIENNE KURT, ZÜRICH (CH); TOMAS DE WOUTERS, ZÜRICH (CH); CHRISTOPHE LACROIX, KILCHBERG (CH); FLORIAN NILS ROSENTHAL, ZÜRICH (CH)

(21) Appl. No.: 17/285,112

(22) PCT Filed: Oct. 15, 2019
(86) PCT No.: PCT/EP2019/078011

§ 371 Date: Apr. 14, 2021

(30) Foreign Application Priority Data
Oct. 15, 2018 (EP) 18200455.6

(51) Int. Cl. C12N 1/20

(52) U.S. Cl. CPC C12N 1/20

(57) ABSTRACT

A method of manufacturing an in vitro assembled consortium of selected bacterial strains by an anaerobic batch co-cultivation is provided. The consortium comprises a plurality of functional groups of the selected bacterial strains. Each functional group performs at least one metabolic pathway of an anaerobic microbiome. Further aspects concern a method of providing an in vitro assembled consortium of selected live, viable bacterial strains and compositions comprising an in vitro assembled consortium.

### TECHNICAL FIELD

The present invention relates to the fields of biotechnology, microbiology and medicine and in particular to a production process for manufacturing consortia of living bacterial strains.

### BACKGROUND ART

The transfer of a fecal microbiota transplant (FMT), i.e. fresh fecal material, from a donor to a patient is known as an effective treatment of intestinal microbiota dysbiosis, particularly of intestinal infections such as CDI (Clostridium difficile infection) and IBD (inflammatory bowel diseases) with a striking efficacy of over 90% for recurrent CDI and fast recovery of bowel function. However, FMT bears significant risks to the patient, due to lack of understanding of compatibility of the patient and the donor's microbiota, that can result in undesired immune reactions and variability in the efficiency of implantation and efficacy of the therapeutic treatment.

WO2018189284 addresses these drawbacks of FMT and provides novel compositions comprising specific consortia of living bacterial strains useful for treatment of intestinal microbiome dysbiosis. These in vitro assembled consortia—in contrast to FMT—correspond to collections of specific and known bacterial strains, in particular of strains providing metabolic functions of a healthy intestinal microbiome. The in vitro assembled consortia are shown to be more efficient and safer in the treatment of dysbiosis and intestinal inflammation, when compared to the traditional FMT therapy. Furthermore, they are suitable for the treatment of a broad range of diseases and disorders.

Zihler et al. 2013 disclose a fermentation-based intestinal model for controlled ecological studies and propose a method to cultivate intestinal microbiomes in their totality starting from fecal material.

Although maintenance of a stable composition in such intestinal microbiome cultivation is possible, the document is silent on the cultivation of in vitro assembled consortia of anaerobic bacteria.

Above-mentioned WO2018189284, the content thereof being incorporated by reference, describes manufacturing of an exemplary in vitro assembled consortium comprising selected bacterial strains by continuous co-cultivation under anaerobic conditions. Continuous co-cultivation conditions, however, are not suitable for an industrial production process of highly standardised products, such as live biological therapeutic products. Because the reproducibility of product quality for products obtained from a continuous cultivation process can hardly be guaranteed to a level that is required for the safety of therapeutic products. Continuous cultivation is susceptible to product variability in particular due to genetic shift of the cultured bacterial strains, batch variations between production batches and intra batch variations during continuous harvesting. In addition, continuous culturing has significant economic drawbacks due to the necessary close monitoring by highly qualified personal for 24-hour operation of bioreactors over several days.

More generally, in the microbiome industry, it is well established how difficult it is to produce a consortium of bacteria at an industrial scale with concerns of reproducibility, yield and robustness.

Current production processes are not designed for the fermentation and subsequent stabilization of strict anaerobic intestinal bacteria since industrial production of bacterial cultures have long been focusing on aerobic or aerotolerant single strain fermentations such as for probiotics. Indeed, consortia often comprise bacteria which are difficult to cultivate together, particularly due to their different requirements for growth or their different growth rates on the same cultivation medium. Therefore, the current industry standard is the production of consortia is the batch-wise production of every single strain of the consortium in under strains specific conditions. In the context of a co-cultivated consortium, it is especially important to prevent the loss of slow growers and/or sensitive bacteria that are often outcompeted when co-cultivated in vitro. Finally, the solutions developed on laboratory scale are not always relevant at the industrial scale and their transposition can be difficult, even sometimes impossible as many intestinal microbes only show limited growth when removed from their intestinal micro-environment and require strain specific, complex media that are strain specific and do not meet industrial production standards for definition of composition or GMP compatibility.

Accordingly, there is a need for a biotechnological production process for efficient and stable multiplication of in vitro assembled consortia comprising selected bacterial strains.

### SUMMARY OF THE INVENTION

Hence, it is a general object of the invention to provide a method for manufacturing a larger quantity of particularly designed in vitro assembled consortia of bacterial strains using a mixed bacterial inoculum, i.e. an inoculum comprising several different bacterial strains, in particular comprising 3 or 4 or 5 or more than 5 strains and comprising in particular up to 10, 15, 20, 50 strains. Multiplication of the bacterial strains used as an inoculum in an anaerobic co-cultivation results in the production of the same in vitro assembled consortium as used for inoculation, i.e. allowing the maintenance and growth of each of the bacteria composing the consortium and the production of metabolites. Thus, the process of manufacture shall ensure that the product of the manufacturing process exhibits the same qualities as the original in vitro assembled consortium that was used as inoculum, in particular with respect to its microbial composition. Thereby, in vitro assembled consortium after its manufacture in a larger quantity shall still provide the same metabolic functions as the original in vitro assembled consortium and accordingly exhibit the same metabolic profile and enable the same therapeutic efficacy as the in vitro assembled consortium used as inoculum for the manufacturing process. Thus, it is an object of the invention to provide a reproducible biotechnological production process for in vitro assembled consortia that ensures a reproducible product quality and efficacy. It is a particular object of the invention to provide a constant product quality of the in vitro assembled consortia as required for products for use in medical therapy. It is a further object of the invention to provide a method of deliberately designing in vitro assembled consortia that can be manufactured in an industrial scale.

These objectives are achieved by methods and applications as outlined in the specification and defined in the independent claims. Preferred embodiments are disclosed in the specification and in the dependent claims.

In a first aspect, the invention concerns a method of manufacturing an in vitro assembled consortium of selected live, viable bacterial strains by an anaerobic co-cultivation in a dispersing medium,

wherein the consortium comprises a plurality of functional groups, each group comprising at least one of the selected bacterial strains,

wherein each functional group of selected bacterial strains performs at least one metabolic pathway of an anaerobic microbiome, in particular of an intestinal microbiome,

wherein the method of manufacturing comprises the steps of

I. providing a sample of the assembled consortium as an inoculum,

wherein the sample of the consortium is obtained from a prior continuous anaerobic co-cultivation process of the selected bacterial strains until a stable microbial profile and a stable metabolic profile characteristic of the in vitro assembled consortium has been established, and

wherein the sample is obtained as a preserved sample;

II. adding the inoculum to the dispersing medium in a bioreactor thereby forming a culture-suspension of the selected bacterial strains;

III. multiplying the selected bacterial strains in the culture suspension by co-cultivation until a stable microbial profile and a stable metabolic profile characteristic of the in vitro assembled consortium is established;

IV. harvesting the consortium of the selected live, viable bacterial strains;

V. optionally, subjecting the harvested consortium to one or more post-treatment steps; characterized in that step III is performed in an anaerobic batch fermentation process or in an anaerobic fed-batch fermentation process.

Particularly, the dispersing medium comprises selected nutrients comprising sugars, starches, fibers and proteins;

Preferably, in step III the criteria (a) and (b), optionally (c) and/or optionally (d) are fulfilled, wherein: according to criteria (a) the selected bacterial strains perform a degradation of the selected nutrients directly, or indirectly via an intermediate metabolite, preferably to an end metabolite, such as a short chain fatty acid, in particular to one or more of acetate, propionate and butyrate;

according to criteria (b) the plurality of functional groups enables metabolic cross-feeding interactions during co-cultivation by comprising a functional group which produces a particular intermediate metabolite and by comprising a functional group consuming said intermediate metabolite, in particular said intermediate metabolite being selected from formate, lactate and succinate;

according to criteria (c) a concentration in the culture-suspension of any intermediate metabolite produced during the degradation is below the concentration inhibiting proliferation of all bacterial strains provided in one of the functional groups; wherein in particular the intermediate metabolite is selected from formate, lactate and succinate;

according to criteria (d) a concentration in the culture-suspension of one or more inhibitory compound produced as a by-product of the degradation, in particular H₂, or a concentration in the culture-suspension of environmental O₂, is below the concentration inhibiting proliferation of all bacterial strains provided in one of the functional groups.

In a second aspect, the invention concerns an in vitro method for manufacturing a consortium of at least three bacterial strains,

wherein each bacterial strain performs at least one metabolic pathway of an anaerobic trophic network, in particular of an intestinal microbiome,

wherein, in said trophic network, the consortium performs a conversion of a substrate into an end metabolite, preferably into a short chain fatty acid, even more preferably selected from acetate, propionate and butyrate, and

wherein the bacterial strains of the consortium are selected to enable metabolic cross-feeding interactions or collaboration between each other during co-cultivation, so as the consortium comprises at least one first bacterium being able to produce an intermediate metabolite and at least one second bacterium which converts said intermediate metabolite, preferably said intermediate metabolite being selected from formate, lactate and succinate;

wherein the method of manufacturing comprises the steps of:

I. providing a sample of the consortium as an inoculum comprising said at least three bacterial strains,

wherein the inoculum is obtained from a prior continuous anaerobic co-cultivation process of the bacterial strains, at least until a stable microbial profile and a stable metabolic profile are obtained, and

wherein the inoculum is provided as a preserved inoculum, preferably a lyophilized or cryopreserved inoculum;

II. adding the inoculum to a culture medium;

III. multiplying the bacterial strains by co-cultivation in the culture medium at least until a stable microbial profile and a stable metabolic profile are obtained, wherein this step is performed in an anaerobic batch or fed-batch fermentation process;

IV. harvesting the consortium of bacterial strains; and

V. optionally, subjecting the harvested consortium to one or more post-treatment or further processing steps.

Preferably, in step 11:
the bacterial strains enable to maintain concentrations in the culture medium of intermediate metabolites of the trophic network, preferably selected from formate, lactate and succinate, below a concentration inhibiting proliferation of at least one bacterial strain of the consortium;
the bacterial strains enable to maintain concentrations in the culture medium of inhibitory by-products of the trophic network, preferably selected from H₂, and O₂, below a concentration inhibiting proliferation of at least one bacterial strain of the consortium.

Preferably in step I, the continuous anaerobic co-cultivation process is preceded by a batch fermentation process.

In particular, the stable microbial profile exhibits an abundance of each of the bacterial strains in the consortium of 10⁵-10¹⁴ 16S rRNA gene copies per ml of the culture suspension or medium, and the stable metabolic profile fulfils one or more of the following criteria:
a concentration of one or more of the intermediate metabolites, preferably selected from formate, lactate and succinate, in the medium is below 15 mM, in particular below 10 mM, 5 mM, 1 mM or more particular below 0.1 mM.
a concentration of one or more of the end metabolites, preferably selected from propionate, butyrate and acetate, is above 5 mM, in particular above 10 mM, more particular above 15 mM, 20 mM or 40 mM.

Preferably, the intermediate metabolite is one or more of formate, lactate and succinate, and the end metabolite is one or more of acetate, propionate and butyrate.

In particular, the stable metabolic profile fulfils one or more of the following criteria:
a concentration of one or more of the intermediate metabolites formate, lactate and succinate in the medium is below 15 mM, in particular below 10 mM, 5 mM, 1 mM or more particular below 0.1 mM.
a concentration of one or more of propionate and butyrate is above 5 mM, in particular above 10 mM, more particular above 15 mM and/or a concentration of acetate is above 10 mM, in particular above 20 mM, more particular above 40 mM.

Preferably, the microbial profile and the metabolic profile are stable during a period of at least 3 days, in particular at least 5 or 7 days.

In particular, the sample of the consortium of step I is selected from a sample preserved by a cryopreservation method or a sample preserved by lyophilisation.

Preferably, the sample of the consortium of step I is cryopreserved in glycerol and wherein the medium of step 11 comprises glycerol as a carbon source, preferably so as to enhance butyrate production.

In particular, the inoculum of step I comprises a sufficient amount of the bacterial strains to achieve a concentration of 10³ to 10¹⁴ 16S rRNA gene copies per ml of the culture-suspension as quantified by qPCR in the bioreactor after addition to the bioreactor in step II and prior to step 11.

Preferably, step III is performed as a fed-batch fermentation process comprising two or more sub-steps of batch cultivation, in particular for a duration of 12 up to 24 or up to 48 hours, wherein between each of the sub-steps a further portion of a dispersing medium providing one or more of the complex compounds, selected from sugars, starches, fibers and proteins is added to the bioreactor and wherein in particular step III is performed as a two-step fed-batch fermentation process comprising the steps of:

III-1 batch fermentation for the duration of one day, in particular for 24 hours, with a dilution of the inoculum into the dispersing medium ranging from 1% to 20% of inoculum to dispersing medium (v/v);

III-2 addition of dispersing medium, in particular the addition of a volume of dispersing equal to the volume of the culture-suspension in the bioreactor;

III-3 continuation of the fermentation for another day, in particular for a further 24 hours.

In one embodiment, during step III or prior to step IV, one or more parameter regarding the microbial profile and/or regarding the metabolic profile of the culture suspension is measured,

wherein optionally the measured value of the one or more parameter is compared to a standard value of said one or more parameter and

wherein the standard value of said one or more parameter corresponds to the value as measured in a culture-suspension comprising the dispersing medium and the selected bacterial strains grown in an anaerobic continuous co-cultivation until said measured value has stabilized over a period of at least 3 days, in particular at least 5 or 7 days.

Preferably, the standard value of the one or more parameter corresponds to a standard value as indicated below:
a concentration of succinate below 15 mM, 10 mM, 5 mM, 1 mM or 0.1 mM
a concentration of formate below 15 mM, 10 mM, 5 mM, 1 mM or 0.1 mM
a concentration of lactate below 15 mM, 10 mM, 5 mM, 1 mM or 0.1 mM
a concentration of acetate above 10 mM, 20 mM or 40 mM
a concentration of propionate above 5 mM, 10 mM or 15 mM
a concentration of butyrate above 5 mM, 10 mM or 15 mM
a redox value below −300 mV, −350 mV or −400 mV,
an optical density above 1.5, 2 or 3
a viability of over 50%, 60% or 70%
an abundance of bacterial strains of 10⁵-10¹⁴ 16S rRNA gene copies per ml

In one embodiment, in step IV, the bacterial strains are harvested during the late exponential phase of growth or at the beginning of the stationary phase of growth.

Preferably, a sample of the consortium harvested in step IV is used directly or is preserved and subsequently used as the inoculum of step I in another round of performing the method according to one of the previous claims.

In a particular aspect, the method according to the invention comprises an additional preparatory stage prior to step I, wherein in the preparatory stage the inoculum of step I comprising the consortium is manufactured from a single-strain sample of each of the bacterial strains of the consortium, wherein said preparatory stage comprises the steps of:

(a) providing single strain samples of the bacterial strains,

(b) inoculating the strains into the dispersing medium in a bioreactor thereby forming a culture suspension and co-cultivating the culture suspension in an anaerobic continuous co-cultivation,

(c) harvesting the consortium of the bacterial strains from the bioreactor after the culture-suspension has established a stable microbial profile and a stable metabolic profile,

(d) optionally subjecting the harvested consortium of the bacterial strains to one or more post-treatment steps.

In a third aspect, the invention concerns an in vitro method for manufacturing an inoculum of at least three bacterial strains,

wherein each bacterial strain performs at least one metabolic pathway of an anaerobic trophic network, in particular of an intestinal microbiome,

wherein, in said trophic network, the consortium performs a conversion of a substrate into an end metabolite, preferably into a short chain fatty acid, even more preferably selected from acetate, propionate and butyrate, and

wherein the bacterial strains of the consortium are selected to enable metabolic cross-feeding interactions or collaboration between each other during co-cultivation, so as the consortium comprises at least one first bacterium being able to produce an intermediate metabolite and at least one second bacterium which converts said intermediate metabolite, preferably said intermediate metabolite being selected from formate, lactate and succinate;

wherein the method of manufacturing comprises the steps of:

(a) providing single bacterial strain samples of the bacterial strains,

(b) inoculating the single bacterial strains into a single culture medium and co-cultivating the bacterial strains in the culture medium by an anaerobic continuous co-cultivation process at least until a stable microbial profile and a stable metabolic profile is reached,

(c) harvesting the bacterial strains, and

(d) subjecting the harvested consortium of the bacterial strains to a preservation treatment, preferably cryopreservation or lyophilisation.

Preferably, in step (b) the anaerobic continuous co-cultivation is preceded by a step of batch fermentation co-cultivation.

Preferably, in step (b)
the bacterial strains enable to maintain concentrations in the culture medium of intermediate metabolites of the trophic network, preferably one or more selected from formate, lactate and succinate, below a concentration inhibiting proliferation of at least one bacterial strain of the consortium;
the bacterial strains enable to maintain concentrations in the culture medium of inhibitory by-products of the trophic network, preferably one or more selected from H₂, and O₂, below a concentration inhibiting proliferation of at least one bacterial strain of the consortium.

In particular, the stable microbial profile comprises an abundance of each of the bacterial strains in the consortium of 10¹-10¹⁴ 16S rRNA gene copies per ml of the culture medium, and the stable metabolic profile comprises:

(i) a concentration of one or more of the intermediate metabolites, preferably selected from formate, lactate, succinate, in the medium is below 15 mM, in particular below 10 mM, 5 mM, 1 mM or more particular below 0.1 mM; and/or

(ii) a concentration of one or more of end metabolites, preferably selected from propionate, butyrate and acetate, is above 5 mM, in particular above 10 mM, more particular above 15 mM, above 20 mM, or above 40 mM.

Preferably, in step (c) the bacterial strains are harvested during the exponential phase of growth or at the beginning of the stationary phase of growth.

Preferably, step (a) comprises the steps of:

(a1) providing and separately cultivating said single strain samples in the presence of a substrate specific for each of said strains thereby obtaining single-strain cultures,

(a2) combining said single-strain cultures of (a1) into a culture-suspension and co-cultivating them under anaerobic conditions in the presence of a dispersing medium,

wherein in particular, the dispersing medium comprises nutrients selected from pectin, arabinogalactan, beta-glucan, soluble starch, resistant starch, fructo-oligosacharides, galacto-oligosacharides, xylan, arabinoxylans, cellulose, yeast extract, casein, skimmed milk, and peptone, wherein in particular a pH value is adjusted within a range of pH 5-7, more particularly a range of pH 5.5-6.5 and

wherein in particular after a duration of 1 or 2 days of co-cultivation half of the volume of the culture-suspension is replaced by the same volume of fresh dispersing medium, and wherein step (a2) is terminated once metabolites succinate, formate and lactate are each below 15 mM.

In particular, in one or both of the optional steps selected from step V and/or step d) of the methods disclosed herein, the harvested consortium is subjected to a preservation-treatment,

wherein the culture-suspension harvested from the bioreactor is handled and stored under protection from oxygen,

wherein the preservation-treatment is selected from cryopreservation and lyophilisation, wherein the post-treatment of cryopreservation comprises the steps of:
mixing the harvested culture-suspension with a cryoprotective solution in particular obtaining a 1:1(v/v) mixture of culture-suspension and glycerol or
centrifuging the harvested culture-suspension and resuspending an obtained pellet in a mixture of the cryoprotective solution and the dispersing medium, in particular in a 1:1 (v/v) mixture of glycerol and the dispersing medium
shock freezing with liquid N₂ or gradually freeze to a storage temperature of at least −20° C., in particular at 20° C. to −80° C.,

wherein the post-treatment of lyophilisation comprises the steps of:
centrifuging the harvested culture-suspension and wash an obtained pellet with a buffer solution
resuspending the pellet in a lyophilisation solution and lyophilise
subsequent storage at a temperature of 4° C. or lower or at room temperature.

Preferably, the sample of the consortium provided as inoculum in step I is a preserved sample of the consortium preserved according to the preservation treatment disclosed above,

wherein a cryopreserved sample of the consortium is thawed at room temperature and inoculated into the bioreactor with an inoculation ratio of 0.1-25% (v/v), in particular with a 0.5-2% (v/v); or

wherein a lyophilised sample of a culture suspension is re-suspended in the dispersing medium and inoculated into the bioreactor with an inoculation ratio of 0.1-25% (v/v), in particular 0.5-2% (v/v); and wherein the total amount of the selected bacterial strains added to the bioreactor in step 11 provides for a concentration of 10³-10¹⁴ 16S rRNA gene copies as quantified by qPCR per ml of the culture suspension in the bioreactor prior to step III.

In a particular aspect, the consortium comprises at least one bacterium for each of functional groups A1 to A9, optionally in combination with one or several bacteria of groups A10 to A15, and wherein functional groups A1 to A15 are:
(A1) Resistant starch degrading formate and acetate producers;
(A2) Starch degrading-, acetate-consuming and butyrate-producers;
(A3) Oxygen-reducing lactate- and formate-producers;
(A4) Starch-reducing lactate- and formate-producers;
(A5) Protein- and lactate-utilizing and propionate-producers;
(A6) Starch-, protein- and lactate-utilizing and butyrate-producers;
(A7) Starch- and protein-degrading formate- and lactate-producers;
(A8) Protein-, succinate-utilizing, and propionate-producers;
(A9) Hydrogen- and formate-utilizing and acetate-producers;
(A10) is an additional functional group of succinate producers;
(A11) Protein—utilizing and acetate and butyrate producers;
(A12) proteins, fibers, starches or sugars consumers and biogenic amines producers such as y-aminobutyric acid (GABA), cadaverine, dopamine, histamine, putrescine, serotonin, spermidine and/or tryptamine producers;
(A13) primary bile acids consumers and secondary metabolite producers;
(A14) vitamins producers such as cobalamin (B12), folate (B9) or riboflavin (B2);
(A15) mucus degraders.

Preferably, the bacterial strains are selected from:

at least one bacterial strain consuming sugars, fibers, and resistant starch, and producing formate and acetate (A1);

at least one bacterial strain consuming sugars, starch and acetate, and producing formate and butyrate (A2);

at least one bacterial strain consuming sugars and oxygen, and producing lactate (A3);

at least one bacterial strain consuming sugars, starch, and carbon dioxide, and producing lactate, formate and acetate (A4);

at least one bacterial strain consuming lactate or proteins, and producing propionate and acetate (A5);

at least one bacterial strain consuming lactate and starch, and producing acetate, butyrate and hydrogen (A6);

at least one bacterial strain consuming sugar, starch, and formate and producing lactate, formate and acetate (A7);

at least one bacterial strain consuming succinate, and producing propionate and acetate (A8); and

at least one bacterial strain consuming sugars, fibers, formate and hydrogen, and producing acetate and optionally butyrate (A9); and

optionally

at least one bacterial strain consuming sugars, fibers, and resistant starch, and producing succinate (A10);

at least one bacterial strain consuming proteins and producing acetate and butyrate (A11);

at least one bacterial strain consuming proteins, fibers, starches or sugars producing biogenic amines such as y-aminobutyric acid (GABA), cadaverine, dopamine, histamine, putrescine, serotonin, spermidine and/or tryptamine (A12);

at least one bacterial strain consuming primary bile acids and producing secondary metabolites (A13);

at least one bacterial strain producing vitamins such as cobalamin (B12), folate (B9) or riboflavin (B2), (A14); and/or

at least one bacterial strain consuming mucus (A15).

More preferably, the bacterial strains comprise:

at least one bacterial strain selected from the genera Ruminococcus, Dorea, Clostridium and Eubacterium (A1);

at least one bacterial strain selected from the genera Faecalibacterium, Roseburia, Anaerostipes and Eubacterium (A2);

at least one bacterial strain selected from the genera Lactobacillus, Streptococcus, Escherichia, Lactococcus and Enterococcus (A3);

at least one bacterial strain of the genus Bifidobacterium or Roseburia (A4);

at least one bacterial strain selected from the genera Clostridium, Propionibacterium, Veillonella, Coprococcus and Megasphaera (A5);

at least one bacterial strain selected from the genera Anaerostipes, Clostridium and Eubacterium (A6);

at least one bacterial strain of the genus Collinsella or Roseburia (A7);

at least one bacterial strain selected from the genera Phascolarctobacterium and Dialister (A8); and

at least one bacterial strain selected from the genera Blautia, Eubacterium and an archaea of the genus Methanobrevibacter or Methanomassiliicoccus (A9);

optionally at least one bacterial strain selected from the genera Alistipes, Bacteroides, Blautia, Clostridium, Ruminococcus and Prevotella (A10); and

optionally

at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;

at least one bacterial strain selected from the genera Clostridium, Coprococcus, Eubacterium, Flavonifractor and Flintibacter (A11);

at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);

at least one bacterial strain selected from the genera Anaerostipes, Blautia, Clostridium and Faecalibacterium (A13)

at least one bacterial strain selected from the genera Bacteroides, Bifidobacterium, Blautia, Clostridium, Faecalibacterium, Lactobacillus, Prevotella and Ruminococcus (A14); and/or

at least one bacterial strain selected from the genera Akkermansia, Bacteroides, Bifidobacterium and Ruminococcus (A15).

Even more preferably, the bacterial strains of the consortium comprise:

at least one bacterium selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1); at least one bacterium selected from Faecalibacterium prausnitzii, Anaerostipes hadrus, Roseburia intestinalis and any combination thereof (A2);

at least one bacterium selected from Lactobacillus rhamnosus, Streptococcus salivarius, Escherichia coli, Lactococcus lactis, Enterococcus caccae, Enterococcus faecalis and any combination thereof (A3);

at least one bacterium selected from Roseburia hominis, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum and any combination thereof (A4);

at least one bacterium selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);

at least one bacterium selected from Anaerostipes caccae, Clostridium indolis, Eubacterium hallii, Eubacterium limosum, Eubacterium ramulus and any combination thereof (A6);

at least one bacterium selected from Roseburia hominis, Collinsella aerofaciens, Collinsella intestinalis, Collinsella stercoris and any combination thereof (A7);

at least one bacterium selected from Phascolarctobacterium faecium, Dialister succinatiphilus, Dialister propionifaciens and any combination thereof (A8); and

at least one bacterium selected from Blautia hydrogenotrophica, Blautia producta, Methanobrevibacter smithii, Candidatus Methanomassiliicoccus intestinalis, Eubacterium limosum and any combination thereof (A9); and
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a preferred aspect, the consortium of bacterial strains comprises: Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6), Collinsella aerofaciens (A7), Phascolarctobacterium faecium (A8), and Blautia hydrogenotrophica (A9) and optionally Bacteroides xylanisolvens (A10).

In another preferred aspect, the consortium of bacterial strains comprises: Ruminococcus bromii (A1), Faecalibacterium prausnitzii (A2), Lactobacillus rhamnosus (A3), Bifidobacterium adolescentis (A4), Anaerotignum (former Clostridium) lactatifermentans (A5), Eubacterium limosum (A6 and A9), Collinsella aerofaciens (A7) and Phascolarctobacterium faecium (A8) and optionally Bacteroides xylanisolvens (A10).

In a fourth aspect, the invention relates to a composition comprising an in vitro assembled consortium of selected live, viable bacterial strains, wherein the consortium is obtainable according to the method according to the invention.

In a fifth aspect, the invention concerns an Inoculum obtainable by a method according to the method according to the invention.

In a sixth aspect, the invention concerns the use of an inoculum according to the invention, for preparing a consortium of viable bacterial strains.

In a seventh aspect, the invention relates to a composition comprising (i) viable bacterial strains and (ii) at least one end metabolite selected from the group consisting of acetate, propionate and butyrate, and mixtures thereof, wherein the composition comprises:

at least one bacterial strain consuming sugars, fibers, and resistant starch, and producing formate and acetate (A1), preferably selected from the genera Ruminococcus, Dorea and Eubacterium;

at least one bacterial strain consuming sugars, starch and acetate, and producing formate and butyrate (A2), preferably selected from the genera Faecalibacterium, Roseburia and Anaerostipes;

at least one bacterial strain consuming sugars and oxygen, and producing lactate (A3), preferably selected from the genera Lactobacillus, Streptococcus, Escherichia, Lactococcus and Enterococcus;

at least one bacterial strain consuming sugars, starch, and carbon dioxide, and producing lactate, formate and acetate (A4), preferably of the genus Bifidobacterium or Roseburia;

at least one bacterial strain consuming lactate or degrading proteins, and producing propionate and acetate (A5), preferably selected from the genera Clostridium, Propionibacterium, Veillonella and Megasphaera;

one Eubacterium limosum strain consuming sugars, fibers, formate, hydrogen, lactate and starch, and producing acetate, butyrate and hydrogen (A6) and (A9),

at least one bacterial strain consuming sugar, starch, and formate and producing lactate, formate and acetate, preferably of the genus Collinsella or Roseburia (A7); and

at least one bacterial strain consuming succinate, and producing propionate and acetate, preferably selected from the genera Phascolarctobacterium and Dialister (A8);

optionally

at least one bacterial strain consuming sugars, fibers, and resistant starch, and producing succinate (A10), preferably selected from the genera Alistipes, Bacteroides, Blautia, Barnesiella, Clostridium, Ruminococcus and Prevotella (A10);

at least one bacterial strain consuming proteins and producing acetate and butyrate (A11), preferably selected from the genera Clostridium, Coprococcus, Eubacterium, Flavonifractor and Flintibacter (A11);

at least one bacterial strain consuming proteins, fibers, starches or sugars producing biogenic amines such as y-aminobutyric acid (GABA), cadaverine, dopamine, histamine, putrescine, serotonin, spermidine and/or tryptamine (A12), preferably selected from the genera Bacteroides, Barnesiella, Bifidobacterium, Clostridium (only tryptamine producers), Enterococcus, Faecalibacterium, Lactobacillus and Ruminococcus (only tryptamine producers) (A12);

at least one bacterial strain consuming primary bile acids and producing secondary metabolites (A13), preferably selected from the genera Anaerostipes, Blautia, Clostridium and Faecalibacterium (A13);

at least one bacterial strain producing vitamins such as cobalamin (B12), folate (B9) or riboflavin (B2), (A14), preferably selected from the genera Bacteroides, Bifidobacterium, Blautia, Clostridium, Faecalibacterium, Lactobacillus, Prevotella and Ruminococcus (A14); and/or

at least one bacterial strain consuming mucus (A15), preferably selected from the genera Akkermansia, Bacteroides, Bifidobacterium and Ruminococcus (A15),

wherein the composition comprises at least 10⁹ bacterial cells per ml and wherein each of the bacterial strains has a viability over 50%, preferably over 70%; and wherein the consortium does not comprise any bacterium from the genus Blautia, especially Blautia hydrogenotrophica, nor an archaea of the genus Methanobrevibacter or Methanomassiliicoccus.

In a eight aspect, the invention concerns a composition comprising (i) viable bacteria strains, and (ii) at least one end metabolite selected from the group consisting of acetate, propionate and butyrate, and mixtures thereof, wherein the composition comprises:

at least one bacterial strain consuming sugars, fibers, and resistant starch, and producing formate and acetate (A1), preferably selected from the genera Ruminococcus, Dorea and Eubacterium;

at least one bacterial strain consuming sugars, starch and acetate, and producing formate and butyrate (A2), preferably selected from the genera Faecalibacterium, Roseburia and Anaerostipes;

at least one bacterial strain consuming sugars and oxygen, and producing lactate (A3), preferably selected from the genera Lactobacillus, Streptococcus, Escherichia, Lactococcus and Enterococcus;

one Roseburia hominis strain consuming sugars, starch, formate and carbon dioxide, and producing lactate, formate and acetate (A4) and (A7);

at least one strain consuming lactate or proteins, and producing propionate and acetate (A5), preferably selected from the genera Clostridium, Propionibacterium, Veillonella and Megasphaera;

at least one strain consuming lactate and starch, and producing acetate, butyrate and hydrogen (A6), preferably selected from the genera Anaerostipes, Clostridium and Eubacterium,

at least one strain consuming succinate, and producing propionate and acetate (A8), preferably selected from the genera Phascolarctobacterium and Dialister; and

at least one strain consuming sugars, fibers, formate and hydrogen, and producing acetate and optionally butyrate (A9); preferably selected from the genera Blautia or Eubacterium; and optionally

at least one bacterial strain consuming sugars, fibers, and resistant starch, and producing succinate (A10), preferably selected from the genera Alistipes, Bacteroides, Blautia, Barnesiella, Clostridium, Ruminococcus and Prevotella (A10);

at least one bacterial strain consuming proteins and producing acetate and butyrate (A11), preferably selected from the genera Clostridium, Coprococcus, Eubacterium, Flavonifractor and Flintibacter (A11);

at least one bacterial strain consuming proteins, fibers, starches or sugars producing biogenic amines such as y-aminobutyric acid (GABA), cadaverine, dopamine, histamine, putrescine, serotonin, spermidine and/or tryptamine (A12), preferably selected from the genera Bacteroides, Barnesiella, Bifidobacterium, Clostridium (only tryptamine producers), Enterococcus, Faecalibacterium, Lactobacillus and Ruminococcus (only tryptamine producers) (A12);

at least one bacterial strain consuming primary bile acids and producing secondary metabolites (A13), preferably selected from the genera Anaerostipes, Blautia, Clostridium and Faecalibacterium (A13);

at least one bacterial strain producing vitamins such as cobalamin (B12), folate (B9) or riboflavin (B2), (A14), preferably selected from the genera Bacteroides, Bifidobacterium, Blautia, Clostridium, Faecalibacterium, Lactobacillus, Prevotella and Ruminococcus (A14); and/or

at least one bacterial strain consuming mucus (A15), preferably selected from the genera Akkermansia, Bacteroides, Bifidobacterium and Ruminococcus (A15),

wherein bacteria strains are present in a total concentration of at least 10⁹ bacteria per ml of composition; and wherein each of the bacteria strains has a viability of over 50%, preferably over 70%.

In a ninth aspect, the invention concerns a composition comprising (i) viable bacteria strains, at least one end metabolite selected from the group consisting of acetate, propionate and butyrate, and mixtures thereof, wherein the composition comprises:

at least one strain consuming sugars, fibers, and resistant starch, producing formate and acetate (A1), preferably selected from the genera Ruminococcus, Dorea and Eubacterium;

at least one strain consuming sugars, starch and acetate, and producing formate and butyrate (A2), preferably selected from the genera Faecalibacterium, Roseburia and Anaerostipes;

at least one strain consuming sugars and oxygen, producing lactate (A3), preferably selected from the genera Lactobacillus, Streptococcus, Escherichia, Lactococcus and Enterococcus;

one Roseburia hominis strain consuming sugars, starch, formate and carbon dioxide, and producing lactate, formate and acetate (A4) and (A7);

at least one strain consuming lactate or proteins, producing propionate and acetate (A5), preferably selected from the genera Clostridium, Propionibacterium, Veillonella and Megasphaera;

one Eubacterium limosum strain consuming sugars, fibers, formate, hydrogen, lactate and starch, and producing acetate, butyrate and hydrogen (A6) and (A9), and

at least one strain consuming succinate, producing propionate and acetate (A8), preferably selected from the genera Phascolarctobacterium and Dialister;

optionally

at least one bacterial strain consuming sugars, fibers, and resistant starch, and producing succinate (A10), preferably selected from the genera Alistipes, Bacteroides, Blautia, Barnesiella, Clostridium, Ruminococcus and Prevotella (A10);

at least one bacterial strain consuming proteins and producing acetate and butyrate (A11), preferably selected from the genera Clostridium, Coprococcus, Eubacterium, Flavonifractor and Flintibacter (A11);

at least one bacterial strain consuming proteins, fibers, starches or sugars producing biogenic amines such as y-aminobutyric acid (GABA), cadaverine, dopamine, histamine, putrescine, serotonin, spermidine and/or tryptamine (A12), preferably selected from the genera Bacteroides, Barnesiella, Bifidobacterium, Clostridium (only tryptamine producers), Enterococcus, Faecalibacterium, Lactobacillus and Ruminococcus (only tryptamine producers) (A12);

at least one bacterial strain consuming primary bile acids and producing secondary metabolites (A13), preferably selected from the genera Anaerostipes, Blautia, Clostridium and Faecalibacterium (A13);

at least one bacterial strain producing vitamins such as cobalamin (B12), folate (B9) or riboflavin (B2), (A14), preferably selected from the genera Bacteroides, Bifidobacterium, Blautia, Clostridium, Faecalibacterium, Lactobacillus, Prevotella and Ruminococcus (A14); and/or

at least one bacterial strain consuming mucus (A15), preferably selected from the genera Akkermansia, Bacteroides, Bifidobacterium and Ruminococcus (A15),

wherein bacteria strains are present in a total concentration of at least 10⁹ bacteria per ml of composition;

and wherein each of the bacteria strains has a viability of over 50%, preferably over 70%.

Preferably, the composition comprises:

at least one bacterium selected from the group consisting of Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);

at least one bacterium selected from the group consisting of Faecalibacterium prausnitzii, Anaerostipes hadrus, Roseburia intestinalis and any combination thereof (A2);

at least one bacterium selected from the group consisting of Lactobacillus rhamnosus, Streptococcus salivarius, Escherichia coli, Lactococcus lactis, Enterococcus caccae and any combination thereof (A3);

at least one bacterium selected from the group consisting of Roseburia hominis, Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum and any combination thereof (A4);

at least one bacterium selected from the group consisting of Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);

one strain of Eubacterium limosum (A6) and (A9);

at least one bacterium selected from the group consisting of Roseburia hominis, Collinsella aerofaciens, Collinsella intestinalis, Collinsella stercoris and any combination thereof (A7); and

at least one bacterium selected from the group consisting of Phascolarctobacterium faecium, Dialister succinatiphilus, Dialister propionifaciens and any combination thereof (A8);

optionally

at least one bacterial strain consuming sugars, fibers, and resistant starch, and producing succinate (A10), preferably selected from the genera Alistipes, Bacteroides, Blautia, Barnesiella, Clostridium, Ruminococcus and Prevotella (A10);

at least one bacterial strain consuming proteins and producing acetate and butyrate (A11), preferably selected from the genera Clostridium, Coprococcus, Eubacterium, Flavonifractor and Flintibacter (A11);

at least one bacterial strain consuming proteins, fibers, starches or sugars producing biogenic amines such as y-aminobutyric acid (GABA), cadaverine, dopamine, histamine, putrescine, serotonin, spermidine and/or tryptamine (A12), preferably selected from the genera Bacteroides, Barnesiella, Bifidobacterium, Clostridium (only tryptamine producers), Enterococcus, Faecalibacterium, Lactobacillus and Ruminococcus (only tryptamine producers) (A12);

at least one bacterial strain consuming primary bile acids and producing secondary metabolites (A13), preferably selected from the genera Anaerostipes, Blautia, Clostridium and Faecalibacterium (A13);

at least one bacterial strain producing vitamins such as cobalamin (B12), folate (B9) or riboflavin (B2), (A14), preferably selected from the genera Bacteroides, Bifidobacterium, Blautia, Clostridium, Faecalibacterium, Lactobacillus, Prevotella and Ruminococcus (A14); and/or

at least one bacterial strain consuming mucus (A15), preferably selected from the genera Akkermansia, Bacteroides, Bifidobacterium and Ruminococcus (A15).

More preferably, the composition comprises:

at least one bacterium selected from the group consisting of Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);

at least one bacterium selected from the group consisting of Faecalibacterium prausnitzii, Anaerostipes hadrus, Roseburia intestinalis and any combination thereof (A2);

at least one bacterium selected from the group consisting of Lactobacillus rhamnosus, Streptococcus salivarius, Escherichia coli, Lactococcus lactis, Enterococcus caccae and any combination thereof (A3); one strain of Roseburia hominis (A4) and (A7);

at least one bacterium selected from the group consisting of Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);

at least one bacterium selected from the group consisting of Anaerostipes caccae, Clostridium indolis, Eubacterium hallii, Eubacterium limosum, Eubacterium ramulus and any combination thereof (A6);

at least one bacterium selected from the group consisting of Roseburia hominis, Collinsella aerofaciens, Collinsella intestinalis, Collinsella stercoris and any combination thereof (A7);

at least one bacterium selected from the group consisting of Phascolarctobacterium faecium, Dialister succinatiphilus, Dialister propionifaciens and any combination thereof (A8); and

at least one bacterium selected from the group consisting of Blautia hydrogenotrophica, Blautia producta, Methanobrevibacter smithii, Candidatus Methanomassiliicoccus intestinalis, Eubacterium limosum and any combination thereof (A9); and
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally at least one bacterium selected from Clostridium butyricum, Coprococcus eutactus, Eubacterium hallii, Flavonifractor plautii and Flintibacter butyricum and any combination thereof (A11);
optionally at least one bacterium selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally at least one bacterium selected from Anaerostipes caccae, Blautia hydrogenotrophica, Clostridium boletae, Clostridium scindens, Clostridium symbiosum and Faecalibacterium prausnitzii and any combination thereof (A13)
optionally at least one bacterium selected from Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally at least one bacterium selected from Akkermansia muciniphila, Bacteroides fragilis, Bacteroides thetaiotaomicron, Bifidobacterium bifidum, Ruminococcus gnavus and Ruminococcus torques and any combination thereof (A15).

Even more preferably, the composition comprises:

at least one bacterium selected from the group consisting of Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);

at least one bacterium selected from the group consisting of Faecalibacterium prausnitzii, Anaerostipes hadrus, Roseburia intestinalis and any combination thereof (A2);

at least one bacterium selected from the group consisting of Lactobacillus rhamnosus, Streptococcus salivarius, Escherichia coli, Lactococcus lactis, Enterococcus caccae, Enterococcus faecalis and any combination thereof (A3);

one strain of Roseburia hominis (A4) and (A7);

at least one bacterium selected from the group consisting of Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);

one strain of Eubacterium limosum (A6) and (A9);

at least one bacterium selected from the group consisting of Phascolarctobacterium faecium, Dialister succinatiphilus, Dialister propionifaciens and any combination thereof (A8); and
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally at least one bacterium selected from Clostridium butyricum, Coprococcus eutactus, Eubacterium hallii, Flavonifractor plautii and Flintibacter butyricum and any combination thereof (A11);
optionally at least one bacterium selected from Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally at least one bacterium selected from Anaerostipes caccae, Blautia hydrogenotrophica, Clostridium bolteae, Clostridium scindens, Clostridium symbiosum and Faecalibacterium prausnitzii and any combination thereof (A13)
optionally at least one bacterium selected from Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally at least one bacterium selected from Akkermansia muciniphila, Bacteroides fragilis, Bacteroides thetaiotaomicron, Bifidobacterium bifidum, Ruminococcus gnavus and Ruminococcus torques and any combination thereof (A15).

Most preferably, the composition comprises: Ruminococcus bromii (A1), Faecalibacterium prausnitzii (A2), Lactobacillus rhamnosus (A3), Bifidobacterium adolescentis (A4), Anaerotignum lactatifermentans (A5), Eubacterium limosum (A6 and A9), Collinsella aerofaciens (A7) and Phascolarctobacterium faecium (A8) and optionally Bacteroides xylanisolvens (A10).

Preferably, the composition is free of, or essentially free of, other viable, live bacteria.

In particular, the composition is free of, or essentially free of intermediate metabolites, preferably selected from the group consisting of succinate, formate and lactate.

In a particular aspect, the composition is for use as a medicament.

Preferably, the composition is for use as a pharmaceutical composition to treat cancer, preferably colorectal cancer, allo-HSCT associated diseases or Graft versus Host Disease (GvHD).

In a particular aspect, the composition is for use in combination with one or more immuno-suppressive or anti-cancer agents.

### FIGURES

The invention will be better understood when consideration is given to the figures and the following detailed description thereof.

FIG. 1 Is a schematic illustration of the key functions of the intestinal microbiome and shows the following functional groups:

(A1) Resistant starch degraders utilizing one or more of the pathways 1,2;

(A2) Starch degrading-, acetate-consuming butyrate-producers utilizing one or more of the pathways 1, 3, 4, 7;

(A3) Oxygen-reducing lactate- and formate-producers utilizing one or more of the pathways 1, 4, 11;

(A4) Starch-reducing lactate- and formate-producers utilizing one or more of the pathways 1, 2, 4;

(A5) Protein- and lactate-utilizing propionate-producers utilizing one or more of the pathways 13, 9;

(A6) Starch-, protein- and lactate-utilizing butyrate-producers utilizing one or more of the pathways 3, 8;

(A7) Starch- and protein-degrading formate- and lactate-producers utilizing one or more of the pathways 1, 2, 4;

(A8) Protein-, succinate-utilizing, propionate-producers utilizing one or more of the pathways 10;

(A9) Hydrogen- and formate-utilizing acetate-producers utilizing one or more of the pathways 6,12;

(A10) is an additional functional group comprising succinate producers utilizing the pathway 5.

An exemplary in vitro assembled consortium is named PB002. PB002 comprises (A1) to (A9). The functional group (A10) is not included in PB002.

Another exemplary in vitro assembled consortium is named PB003. It comprises all of the functional groups included in PB002 except A8 and comprises the additional strains C. scindens and B. fragilis of the functional groups A1 and A10 respectively, i.e. 10 strains as further described regarding FIG. 6.

FIG. 2: Stabilization of a plurality of functional groups in a bioreactor using continuous fermentation: Short chain fatty acid concentrations in a 300 ml bioreactor during establishment and stabilization of the exemplary bacterial consortium PB002 comprising a plurality of functional groups encompassing A1 to A9. The inoculum for the bioreactor was prepared in two steps, first obtaining a single strain culture of the selected bacterial strains of each functional group, cultivating each strain for 48 h in an individually adapted dispersing medium, followed by a mixing of the single strain cultures and co-cultivating under anaerobiosis for obtaining the inoculum. The x-axis indicates the time in days starting at day 0 for inoculation of the bioreactor. The y-axis represents the concentration of the quantified metabolites in mM of of acetate ( [symbol] ), propionate ( [symbol] ), butyrate ( [symbol] ), formate ( [symbol] ), lactate ( [symbol] ) and succinate ( [symbol] ). The results show that after two batch fermentations to prepare the inoculum comprising the plurality of the selected strains, it takes 7 days of continuous fermentation to reach a steady state, i.e. an equilibrium, in which all desired metabolites are produced at the desired concentration and no intermediate metabolites are accumulated. This indicates that intermediate metabolites produced by some of the selected functional groups are consumed by other selected functional groups. End-metabolites are at the targeted ratios confirming the quality of the stable consortium.

FIG. 3: Establishment of a plurality of functional groups in a continuous fermentation using cryopreserved inoculum: Initial stabilization phase of a bioreactor inoculated with stored reactor effluent (−20° C.) from a previous continuous co-cultivated fermentation of the exemplary consortium PB002 results in a fast stabilization of the continuous fermentation. All bacterial strains and the desired interactions were fully established after 4 days of fermentation already resulting in a stable production of the desired end metabolite (acetate, propionate, butyrate) as well as a successful consumption of intermediate metabolites (formate, lactate) to end metabolites that are comparable to the values of the previous fermentation used to produce the inoculum (time points −3 to −1). The x-axis indicates the time in days starting at day 0 for inoculation of the bioreactor. The y-axis represents the concentration of metabolites in mM of acetate ( [symbol] ), propionate ( [symbol] ), butyrate ( [symbol] ), formate ( [symbol] ), lactate ( [symbol] ), and succinate ( [symbol] ).

These data show that a continuously co-cultivated consortium of functional groups harvested as reactor effluent, preserved by cryopreservation and stored at a temperature of −20° C. or lower, e.g. −80° C. can be used directly as inoculum in a subsequent manufacturing process to obtain more of the same consortium. Thereby simplifying the subsequent manufacturing process. The steps of obtaining a single strain culture of the selected bacterial strains, cultivating each strain for 48 h in an individually adapted dispersing medium, followed by a mixing of the single strain cultures under anaerobiosis for inoculation can be replaced by a cryopreserved inoculum comprising the plurality of selected strains preserved as a co-cultivated consortium as shown with the exemplary consortium PB002.

FIG. 4: Establishment of a plurality of functional groups in a continuous fermentation using preserved consortia: Measured metabolite concentration of continuously co-cultured exemplary consortium PB002 in the bioreactor supernatant at day 7 after inoculation. The tested groups include: (1) control reactor inoculated with mix of independently cultured fresh cultures of the 9 strains contained in PB002 (prepared in two steps as described in FIG. 2 above); (2) bioreactor inoculated with cryopreserved PB002, stored for 3 month at −20° C. in a cryoprotective glycerol solution; (3) bioreactor inoculated with a mix of the 9 single strains of PB002 stored independently for 3 months in the described glycerol solution and mixed before inoculation after thawing; (4) bioreactor inoculated with 6-month-old lyophilised PB002 stored at 4° C. and re-suspended in the dispersing medium for inoculation; (5) bioreactor inoculated with a mix of 6-month-old independently lyophilised cultures of the 9 strains contained in PB002. Column 2 and 4 represent bioreactors inoculated with the cryopreserved PB002 consortium and the lyophilised PB002 consortium, respectively, using the stable PB002 consortia after co-cultivation for preservation such as described in FIG. 2 above. Metabolites are represented in mM of acetate ( [symbol] ), propionate ( [symbol] ), butyrate ( [symbol] ), succinate ( [symbol] ), lactate ( [symbol] ), formate ( [symbol] ). After 7 days of continuous cultivation as described in FIG. 2, the bioreactors using the co-cultured and stored PB002 suspensions (2) and (4) as inoculum showed presence of all major end metabolites, acetate, propionate and butyrate in correct ratios compared to the control reactor (1). In the bioreactors inoculated with the strains of PB002 independently stored as single strains and mixed prior to inoculation (3) and (5) did not result in the desired metabolic profiles as compared the control (1) indicating the lack of establishment of all functional groups within the consortium PB002.

These data show that cryopreservation and lyophilisation of a stable consortium as produced in example 3 maintains the metabolic profile of the stable consortium after the preservation and storage process, including the thawing or rehydration process for the lyophilised consortium, respectively, and results in rapid re-establishment of all functional groups within the consortium PB002 after storage resulting in the metabolic profile characteristic of the stable consortium previous to conservation during subsequent anaerobic co-cultivation. Consortia stored after continuous co-cultivation exhibit an increased stress-resistance when preserved by lyophilisation or cryopreservation as compared to the single strains of the consortium preserved and stored separately.

FIG. 5: Metabolic profiles in anaerobic co-cultivation of the exemplary in vitro assembled consortium PB002 when preserved as a previously co-cultured consortium comprising the plurality of functional groups and all of the selected strains versus the metabolic profiles in anaerobic co-cultivation from inoculation with the collection of all of the selected strains wherein each of the strains was individually preserved. Absolute abundances of all strains of the continuously cultured consortium PB002 at day 7 after inoculation. The tested groups include: (1) control reactor inoculated with a mix of independently cultured fresh cultures of the 9 strains of PB002 (prepared in the two steps (a1) and (a2) as described above); (2) bioreactor inoculated with cryopreserved PB002, stored for 3 month at −20° C. in a cryoprotective glycerol solution; (3) bioreactor inoculated mix of the 9 single strains contained in PB002 stored independently for 3 months in the glycerol solution and mixed before inoculation after thawing; (4) bioreactor inoculated with 6-month-old lyophilised PB002 stored at 4° C. and re-suspended in the dispersing medium for inoculation; (5) bioreactor inoculated with a mix of 6-month-old independently lyophilised cultures of the 9 strains in contained in PB002. Abundances of each strain representing a functional group were quantified using specific qPCR primers as described in example 4 and are indicated in copies of the gene/ml of culture for the strains representing A1 ( [symbol] ), A2 ( [symbol] ), A3 ( [symbol] ), A4 ( [symbol] ), A5 ( [symbol] ), A6 ( [symbol] ), A7 ( [symbol] ), A8 ( [symbol] ), and A9 ( [symbol] ). Error bars represent standard deviations of 2 technical replicates. Two-way ANOVA was performed. The figure shows qPCR quantification of the different strains representing the plurality of functional groups in each reactor at day 7 after inoculation. ⁽*⁾ indicates a significant change in abundance of the relative abundance of a functional group for the bioreactors (2), (3), (4) and (5) as compared to the control reactor (1). Significance is defined with a p-value <0.05 based on two-way ANOVA analysis.

The data demonstrate that the strains individually preserved by cryopreservation or lyophilisation used for inoculation of reactors 3 and 5, respectively when used as inoculum for co-cultivation do not establish themselves at the desired abundance characteristic of the exemplary stable consortium PB002 as shown in the control reactor (1). For example (3) and/or (5) deviate significantly from (1) for the following functional groups A1, A2, A4, A5, A9, with some of the selected bacterial strains missing entirely. In contrast, the use of an inoculum produced by preservation of the selected strains after co-cultivation as a stable consortium using cryopreservation (2) or lyophilisation (4), respectively, show the establishment of all functional groups A1 to A9 at comparable levels to the control reactor (1).

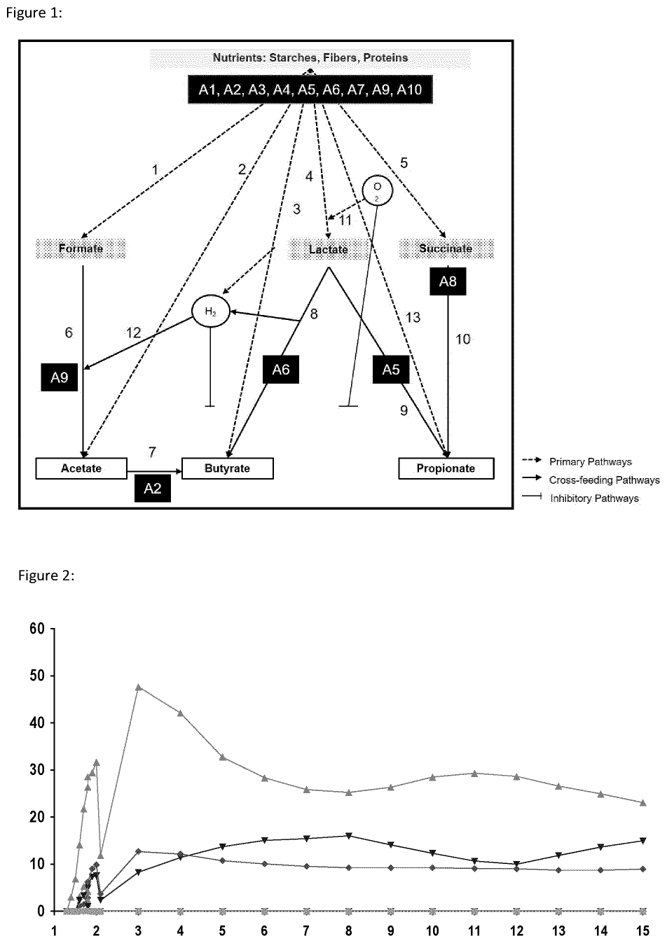

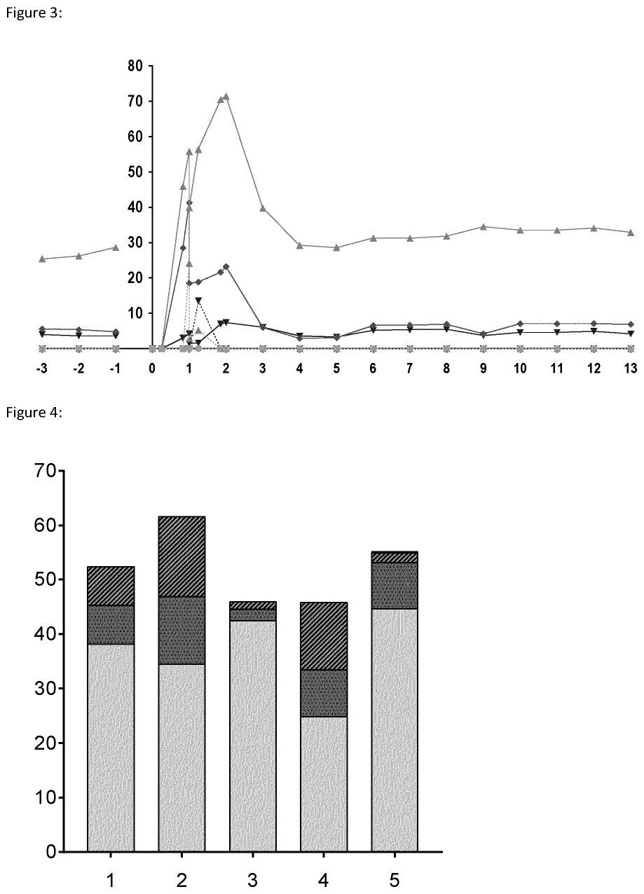

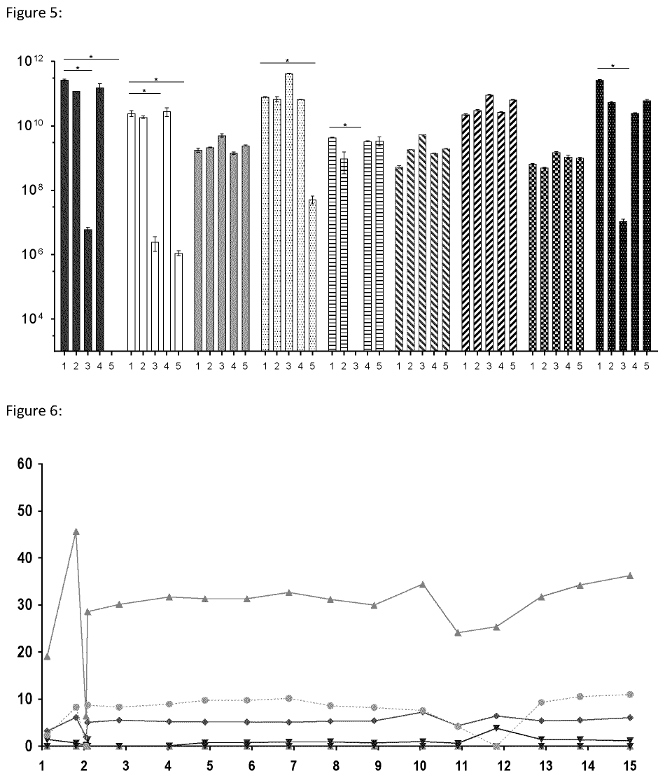

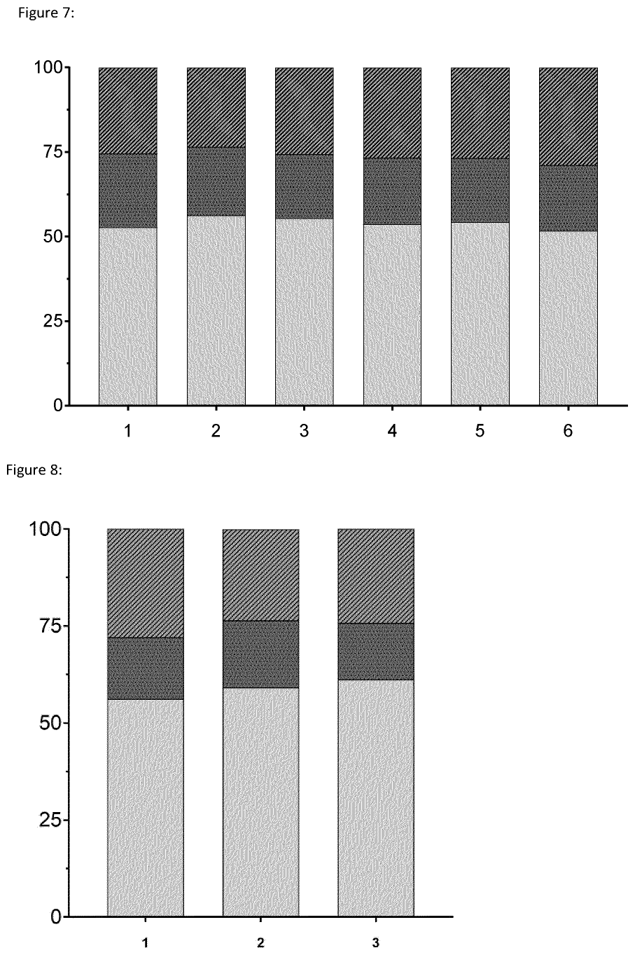

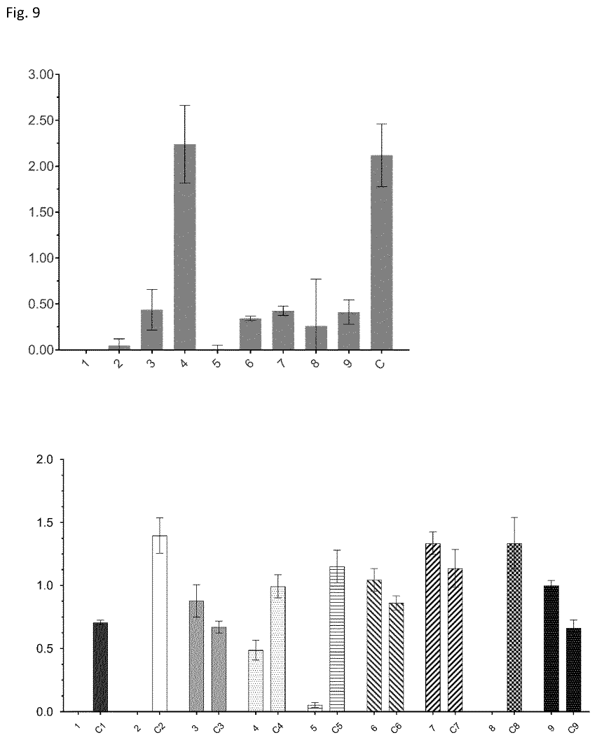

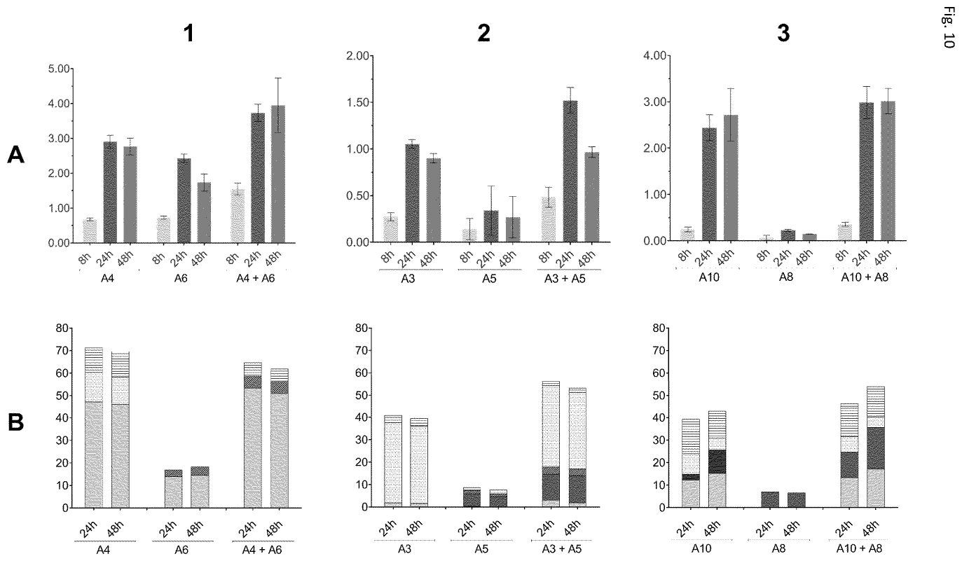

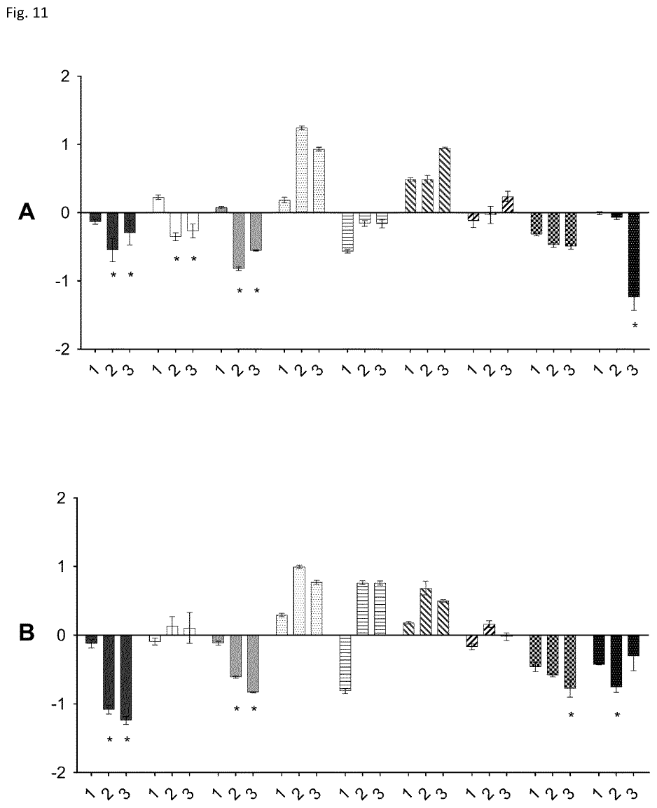

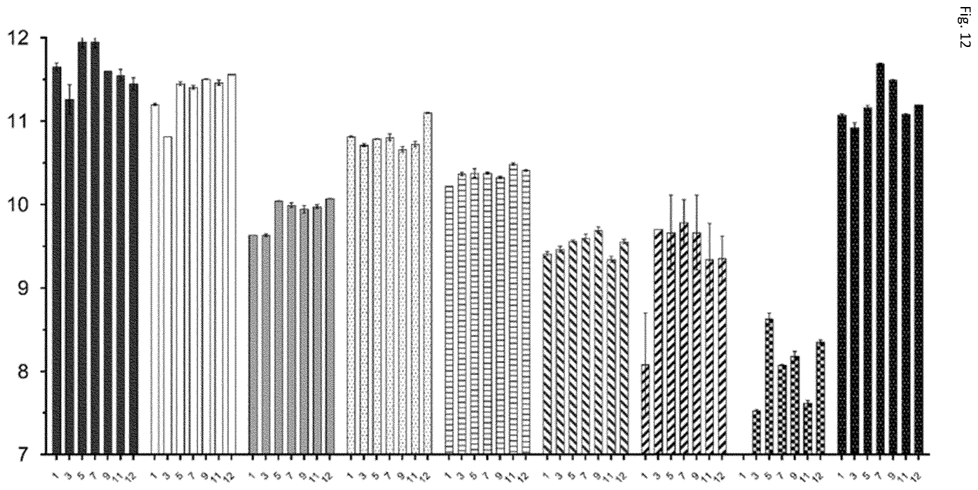

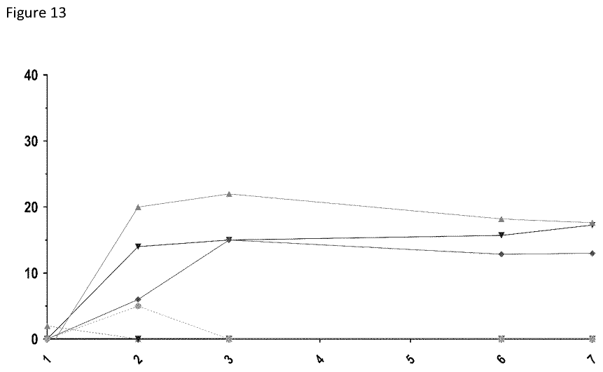

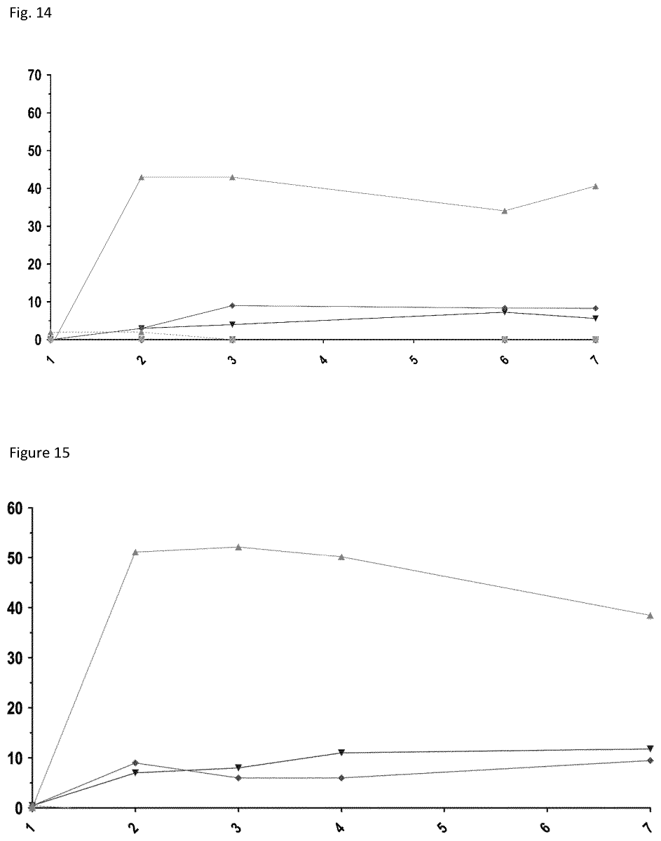

FIG. 6: Maintenance of the plurality of functional groups in consortium PB003: Metabolite concentrations in a 300 ml bioreactor during establishment and stabilization of an exemplary bacterial consortium consisting of functional groups A1 to A7 and A9 to A10 using 10 strains (two strains of functional group A1 were used). The x-axis indicates the time in days starting at day 0 for inoculation of the bioreactor. The y-axis represents the concentration of metabolites in mM of acetate ( [symbol] ), propionate ( [symbol] ), butyrate ( [symbol] ) formate ( [symbol] ), lactate ( [symbol] ), and succinate ( [symbol] ), This is an exemplary embodiment of a stable, in vitro assembiej consortium of a plurality of functional groups derived according to example 1 using the method described in example 2. The stabilized consortium shows a metabolic profile according to the scheme in FIG. 1, with the end-metabolites acetate and butyrate at desired stabilizing close to 30 mM and 5 mM respectively and non-inhibiting concentrations of succinate stabilizing close to 10 mM.

The data demonstrate a successful application of the approach in FIG. 1 proving the concept of assembling according to functional groups.

FIG. 7: Maintenance of the plurality of functional groups in the preserved inoculum of PB002:

Relative concentrations of metabolites in the culture suspension of continuously co-cultured exemplary consortium PB002 in six different bioreactors at day 8 after inoculation with cryopreserved or lyophilised inocula produced from co-cultivated PB002. (1) to (3) are the metabolic profiles of three independent bioreactors inoculated with cryopreserved PB002 inocula stored for at least 3 months at −20° C. in glycerol solution; (4) to (6) are the metabolic profiles of three independent bioreactors produced by inoculation with lyophilised PB002 inocula, stored at 4° C. for at least 3 months. All used inocula of PB002 (cryopreserved and lyophilised) were produced under continuous fermentation for at least 8 days prior [symbol] to cryopreservation/lyophilisation and storage as described in example 3. Metabolites are represented as % of the total bacterial metabolites produced; acetate ( [symbol] ), propionate ( [symbol] ), butyrate ( [symbol] ), succinate ( [symbol] ), lactate ( [symbol] ), formate ( [symbol] ). The co-cultured PB002 suspensions showed presence of all desired end metabolites, acetate, propionate and butyrate in comparable ratios, reproducible among the different bioreactors and independent of the stabilization procedure.

The data demonstrate the reproducible maintenance of the plurality of functional groups resulting in the desired the metabolic profile for the exemplary consortium PB002 when co-cultivated using the cryopreserved or lyophilised inocula of PB002 produced under continuous fermentation for at least 8 days prior to cryopreservation or lyophilisation and storage as described in example 3.

FIG. 8: Use of preserved consortium for batch fermentation of a plurality of functional groups: Mean bacterial metabolite concentration of co-cultured exemplary consortium PB002 in three different bioreactors after 48 h of batch fermentation inoculated with lyophilised PB002 consortium. (1) to (3) were produced by inoculation of a bioreactor with three individually lyophilised PB002 inocula, stored at 4° C. for at least 3 months. PB002 inocula were produced under continuous fermentation conditions for at least 8 days before lyophilisation and storage. Metabolites are represented as relative abundances of total bacterial metabolites [%] produced; acetate ( [symbol] ), propionate ( [symbol] ), butyrate ( [symbol] ), succinate ( [symbol] ), lactate ( [symbol] ), formate ( [symbol] ).

After 48 h of batch cultivation all three repetitions showed the presence of all major end metabolites, acetate, propionate and butyrate in physiologically relevant ratios (Chassard and Lacroix, 2013). These data demonstrate a) the reproducibility of the establishment of the plurality of functional groups and the resulting metabolic profile of the exemplary PB002 consortium when inoculated with a lyophilised inoculum produced as described in example 3 and b) the establishment of the desired plurality of functional groups of the exemplary consortium PB002 in a batch fermentation after 48 h of anaerobic batch cultivation when starting from a preserved inoculum of the stable exemplary consortium PB002, demonstrating a significant advantages for biotechnological production of stable consortia, in particular for use in medical therapy as compared to the use of continuous cultivation.

FIG. 9: Growth of strains and consortium on medium as measured by optical density (OD600) and strains specific qPCR of the medium inoculated with the single strains of PB002 (1-9) and co-cultured PB002 (C) were performed in Hungate tubes containing 3-times buffered PBMF009 fermentation medium. Individual tubes were inoculated in triplicate with 0.8 mL of a 1:10 dilution of 48 h old cultures or 0.8 mL of a 1:10 dilution of effluent from a continuously operated bioreactor producing PB002 (day 15 of fermentation). Abundances of each strain representing a functional group were quantified using specific [symbol] qPCR primers as described in example 4. The numbers indicated correspond to the increase in log 10 copies of 16S rRNA gene/ml of culture for the strains representing A1 ( [symbol] ), A2 ( [symbol] ), A3 ( [symbol] ), A4 ( [symbol] ), A5 ( [symbol] ), A6 ( [symbol] ), A7 ( [symbol] ), A8 ( [symbol] ), and A9 ( [symbol] ). No bar indicates no detectable growth.

FIG. 10: Co-cultures of 2 strains with expected cross feeding behavior. Optical density values (OD600) (A) and bacterial metabolite concentrations (B) of single and co-cultures of:
B. adolescentis (A4) and E. limosum (A6) (lactate/formate-producer and lactate/formate-consumer/butyrate-producer) (1);
Lb. rhamnosus (A3) and A. lactatifermentans (A5) (lactate-producer and lactate-consumer/propionate producer) (2); and
B. xylanisolvens (A10) and P. faecium (A8) (succinate-producer and succinate-consumer/propionate producer) (3),

were performed in Hungate tubes containing YCFA-Starch medium. Individual tubes were inoculated in triplicate with 0.3 mL of 48 h old cultures at an OD of 1.0. Metabolites are represented in mM of acetate ( [symbol] ), propionate ( [symbol] ), butyrate ( [symbol] ), succinate ( [symbol] ), lactate ( [symbol] ), formate ( [symbol] ). [symbol] FIG. 11: Microbial profiles in anaerobic co-cultivation of the exemplary in vitro assembledconsortium PB002 after 48 h of batch fermentation (production process, prepared by inoculating the inoculum produced in step 1 as described in the example 11). The graph shows the absolute difference in abundance compared to the desired composition. The desired composition represents the relative abundance of co-cultured strains at the point of inoculum preservation. The tested groups include

The difference in relative abundance to the desired composition were quantified using specific qPCR primers as described in example 4 and are indicated in copies of the log 10 16S rRNA gene/ml of culture for the strains representing A1 ( [symbol] ), A2 ( [symbol] ), A3 ( [symbol] ), A4 ( [symbol] ), A5 ( [symbol] ), A6 ( [symbol] ), A7 ( [symbol] ), A8 ( [symbol] ), and A9 ( [symbol] ). Error bars represent standard deviations of 3 technical replicates. Two-way ANOVA was performed. Significance (*) is defined with a p-value <0.05.

FIG. 12: Microbial profiles in anaerobic co-cultivation of the exemplary in vitro assembled consortium PB002 and presence of all functional groups throughout 12 weeks of continuous fermentation. The figure shows absolute abundances of all strains representing the plurality of functional groups of the continuously cultured consortium PB002 over a period of 12 weeks. The reactor was inoculated with a mix of independently cultured fresh cultures of the 9 strains of PB002. Abundances of each strain representing a functional group were quantified using specific qPCR primers as described in example 4 and are indicated in copies of the log 10 16S rRNA gene/ml of culture for the strains representing A1 ( [symbol] ), A2 ( [symbol] ), A3 ( [symbol] ), A4 ( [symbol] ), A5 ( [symbol] ), A6 ( [symbol] ), A7 ( [symbol] ), A8 ( [symbol] ), and A9 ( [symbol] ). Error bars represent standard deviations of 2 technical replicates.

FIG. 13: Establishment of PB002 with alternative strains (i.e. PB004). The x-axis indicates the time in days starting at day 0 for inoculation of the bioreactor. The y-axis represents the concentration of the quantified metabolites in mM of acetate ( [symbol] ), propionate ( [symbol] ), butyrate ( [symbol] ), formate ( [symbol] ), lactate ( [symbol] ), and succinate ( [symbol] ).

FIG. 14: Establishment of PB010 a consortium combining two functional groups (A6 and A9) into one single bacterium. The x-axis indicates the time in days starting at day 0 for inocul r₀% ff bioreactor. The y-axis represents the concentration of the quantified metabolites in mM of acetate ( [symbol] ), propionate ( [symbol] ), butyrate ( [symbol] ), formate ( [symbol] ), lactate ( [symbol] ), and succinate ( [symbol] ).

FIG. 15: Establishment of PB011 consisting of functional groups A1 to A10. The x-axis indicates the time in days starting at day 0 for inoculation of the bioreactor. The x-axis indicates the time in days starting at day 0 for inoculation of the bioreactor. The y-axis represents the concentration of the quantified metabolites in mM of acetate ( [symbol] ), propionate ( [symbol] ), butyrate ( [symbol] ), formate ( [symbol] ), lactate ( [symbol] ), and succinate ( [symbol] ).

### DISCLOSURE OF THE INVENTION

### Definitions

As used herein, the term “a”, “an”, “the” and similar terms used in the context of the present invention (especially in the context of the claims) are to be construed to cover both the singular and plural unless otherwise indicated herein or clearly contradicted by the context.

As used herein, the terms “including”, “containing” and “comprising” are used herein in their open, non-limiting sense.

The terms “microbiome” and “microbiota” are known as synonyms and particularly denote the totality of microbial life forms within a given habitat or host. The term “intestinal microbiome” in particular refers to the gut microbiota.

The terms “bacteria” and “bacterial strain” are known and particularly denote the totality of the domain bacteria. Due to their function, also the genera Methanobrevibacter and Candidatus Methanomassiliicoccus of the domain archaea shall be included in the term “bacteria” as used in this text.

The terms “viable bacteria” and/or “live bacteria” are known in the field; in particular, they denote bacteria, wherein viable bacterial strains have the capacity to grow under suitable conditions and live bacterial indicate viability as measured using biochemical assays. The term viable, live bacterial strains in particular relates to bacterial strains (i) having a viability of over 50% (e.g. in pharmaceutical products), typically over 60% such as over 90% (e.g. in products manufactured according to the inventive method) as determined by flow cytometry. Viability over 90% is typically observed in the compositions as initially obtained by continuous cultivation and by batch or fed-batch cultivation, viability over 60% is typically observed after stabilization.

It should be noticed that bacterium Clostridium lactatifermentans has been recently renamed Anaerotignum lactatifermentans. Then, as used herein the terms “Clostridium lactatifermentans” and “Anaerotignum lactatifermentans” have the same meaning and can be used interchangeably.

The term “consortium”, “microbial consortium” or “bacterial consortium” refers herein to at least three microbial organisms, preferably officiating in the same metabolic or trophic network. As such, microbial members of the consortium collaborate, preferably for their subsistence into the consortium. Even though a consortium according to the invention is based on bacteria, the consortium disclosed herein does not rely on a particular composition of specific bacteria or bacterial strains but by the functions or capacities of such bacteria, especially functions that allow their interaction and maintenance in the consortium. Assembly of a consortium based on functional groups is more particularly defined hereafter.

The term “functional group” as used herein, refers to functions or capacities fulfilled by bacteria. Such functions are for example capacity to degrade or convert a particular substrate, for example such as starch, and to produce a particular product or metabolite, for example such as butyrate. Generally, one bacterium is able to degrade or convert a substrate (e.g. starch) and to produce a product (e.g. butyrate).

Then, a functional group comprises bacteria that are able to degrade or convert the same substrate(s) (e.g. starch) and to produce the same metabolite(s) (e.g. butyrate); i.e. bacteria that are able to perform similar metabolic pathways.

The term “metabolic pathway” refers to a reaction that can be performed by a bacterium or occurring within a bacterium. In most cases of a metabolic pathway, substrates, products and optionally intermediates are processed through enzymatic reactions. A metabolic pathway converts a substrate into a product. A metabolic pathway can be carried out by the same enzymatic reaction(s) or by different ones. Metabolic pathways are generally included in a metabolic network, the product of one reaction is generally acting as the substrate for the next one. In the context of the invention, substrate can be for example starch, resistant starch, phenolic compounds, amino acids, proteins and/or fibers; and the product can be intermediate metabolites such as sugar monomers, amines, formate, lactate and succinate; or end metabolites such as acetate, butyrate and propionate; or gas, such as hydrogen, carbon dioxide, methane, sulfur containing gas or oxygen.

The terms “metabolic network” or “trophic network” as used herein refer to a set of metabolic and physical processes that rely on metabolic pathways that are interconnected. Such connexions of metabolic pathways allow the bacteria of a consortium to mutually promote growth through interaction, especially via cross-feeding, to form a collaborative network in which all of the bacteria are viably maintained in ratios defined by the interaction.

The term “beginning of the stationary phase of growth” refers to a stage of growth that immediately follows the exponential or logarithmic (log) phase of growth. It particularly refers to the phase where the exponential phase begins to decline as the available nutrients become depleted and/or inhibitory products start to accumulate. In this period, the number of living bacteria starts to remain constant in the culture.

The term “dysbiosis” is known and denotes the alteration of the microbiota in comparison to the healthy state. The microbiota's state may be characterized by determining key markers, intermediate metabolites and end metabolites. The healthy microbiota is characterized by the absence of intermediate metabolites. Accordingly, a stable state characterized by accumulation of intermediate metabolites is referred to as dysbiosis.

The term “treatment” refers to any act intended to ameliorate the health status of patients or subjects such as therapy, prevention, prophylaxis and retardation of a disease. It designates both a curative treatment and/or a prophylactic treatment of a disease. A curative treatment is defined as a treatment resulting in a cure or a treatment alleviating, improving and/or eliminating, reducing and/or stabilizing the symptoms of a disease or the suffering that it causes directly or indirectly. A prophylactic treatment comprises both a treatment resulting in the prevention of a disease and a treatment reducing and/or delaying the incidence of a disease or the risk of its occurrence. In certain embodiments, such term refers to the improvement or eradication of a disease, a disorder or symptoms associated with it.

The term “organic acid” is known and denotes organic compounds with acidic properties.

The term “short chain fatty acids” (SCFA) is also known as volatile fatty acids (VFAs) and specifically denotes fatty acids with two to six carbon atoms.

The term “intermediate metabolite” denotes the metabolites produced by members of the microbiota that are used as energy source by other members of the microbiota. Such intermediate metabolites in particular may include degradation products from fibers, proteins or other organic compounds, but also formate, lactate and succinate that are typical intermediate products of known metabolic pathways. They are not found in healthy individuals. In particular, they are typically not enriched in the feces of a healthy individual. More generally, the term “intermediate metabolites” may refer to an undesirable metabolite, the presence or amount of which being limited as much as possible in the final product and/or patient.

The term “end metabolites” refers to metabolites found in healthy individuals. In particular, “end metabolites” may denote the metabolites produced by the intestinal microbiota that are not utilized or only partially utilized by other members of the microbiota. End metabolites in particular include the short chain fatty acids acetate, propionate and butyrate comprising two, three and four carbon atoms, respectively. They are partially absorbed by the host and partially secreted in the feces. More generally, the term “end metabolites” may refer to a wanted metabolite, the presence or amount of which being promoted in the final product.

The term “metabolic profile” as used herein refers to the expression of metabolic pathways and particularly to the presence or amount of particular metabolites produced by a bacterium or a consortium from a particular substrate. This metabolic profile can be monitored through time by any technique known by the man skilled in the art, preferably to monitor the production, quantity or amount of metabolites that are produced by a bacterium or consortium. For example, bacteria can be characterized for growth and metabolite production on M2GSC Medium (ATCC Medium 2857) and modifications thereof where the carbon sources such as glucose, cellobiose and starch are replaced by specific substrates including intermediate metabolites and/or fibers, preferably such as those found in the human intestine. The concentrations of the produced metabolites can for example be quantified by any analytic method known by the person skilled in the art, for instance refractive index detection high pressure liquid chromatography (HPLC-RI; for example, as provided by Thermo Scientific Accela™). By “stable metabolic profile”, it is meant that the production and/or quantity of produced metabolites does not significatively vary through time, for example during a period of at least 2, 3, 4, 5, 6, 7, 8, 9 or 10 days; and/or that the variation does not exceed a factor 2, 5 or 10, or does not exceed 2, 5, 10, 15, 20 or 25% of a standard value, preferably such standard value being the average quantity of metabolite measured over time, for example during a period of at least 2, 3, 4, 5, 6, 7, 8, 9 or 10 days, preferably 3 days. When several metabolites are taken into consideration, a stable metabolic profile may refer to a ratio between the metabolites that does not significatively vary through time, for example during a period of at least 2, 3, 4, 5, 6, 7, 8, 9 or 10 days and/or the variation does not exceed 2, 5, 10, 15, 20 or 25% of a standard ratio, preferably such standard ratio being the average ratio between metabolites measured over time, for example during a period of at least 2, 3, 4, 5, 6, 7, 8, 9 or 10 days, preferably 3 days. A “stable metabolic profile” particularly refers to the production and/or the quantity of an end metabolite, such as acetate, butyrate or propionate, in a similar amount during a certain time. Additionally, or alternatively, it refers to the production and/or the quantity of intermediate metabolites, such as formate, lactate and succinate, in a similar amount during a certain time.

The term “microbial profile” as used herein refers to the content or number of bacteria in a sample. It particularly refers to the presence, absence and/or number of bacteria in a sample, preferably in a sample comprising the consortium of the invention. The person skilled in the art knows how to establish a microbial profile, for example via 16S RNA sequencing. A “stable microbial profile” particularly refers to the presence and/or number of bacteria that does not significatively vary through time, for example during a period of at least 2, 3, 4, 5, 6, 7, 8, 9 or 10 days, preferably 3 days, or that only slightly vary, preferably such variation does not exceed a factor 2, 5 or 10, nor 2, 5, 10, 15, 20, 25, 30, 40, 50 or 60% of a standard value, preferably such standard value being the average quantity of bacteria measured over time, for example during a period of at least 2, 3, 4, 5, 6, 7, 8, 9 or 10 days, preferably 3 days.

As used herein a “stable inoculum” or “stabilized inoculum” refers to an inoculum of bacteria, preferably an inoculum of a consortium according to the invention, having a stable metabolic profile and/or a stable microbial profile. A “stable consortium” refers to a consortium of bacteria having a stable metabolic profile and/or a stable microbial profile.

The term “substrate” is known and encompasses “nutrients” and other components of the dispersing medium supporting proliferation of one or more bacterial strain. The term “nutrient” in this text particularly refers to a component of the dispersing or culture medium that some bacterial strains are capable of metabolizing, i.e. nutrients that can be converted into metabolites or energy. In some embodiments, the term substrate encompasses intermediate metabolites produced by one member of the consortium, so that intermediate metabolites as substrate does not necessarily need to be added to the culture medium. Then a bacterial strain can use intermediate metabolites as substrate, especially to produce end metabolites.

The term “fiber” is known and denotes in this text any carbohydrate polymer with more than ten monomeric units and refers in particular to plant fibers, modified plant fibers and dietary fibers. Fibers are generally not completely hydrolysed in the small intestine of humans. Exemplary fibers include e.g. waxes, lignin, polysaccharides, such e.g. as cellulose, starch, resistant starch and pectin.

The term “effluent” is known and particularly denotes the outflow of a continuous fermentation process containing consumed growth medium, bacteria and bacterial metabolites.

The term “inhibitory concentration” is known in the art and refers to a concentration of a compound, such as an intermediate metabolite or a gas, that inhibits or decreases the proliferation, the growth and/or the metabolic production or activity of a bacterium.

The term “preserved sample” as used herein refers to a sample that has been subjected to one or more treatment for preservation or care of the sample. Preferably, such treatment enables to preserve the stability and/or the viability of a bacterial strain. In one embodiment, the treatment may include the addition of a stabilization solution or agent.

The term “fermentation” is known and in the context of this text refers to an anaerobic process of cultivating microbes, preferably based on predominantly anaerobic respiration, in particular of cultivating bacterial strains in a bioreactor comprising a liquid dispersing or cultivation medium. Fermentation in particular denotes an enzymatically controlled anaerobic metabolism of energy-rich compounds.

The term “batch fermentation” is known and denotes a fermentation process in a bioreactor, wherein during the fermentation process no material is removed from nor added to the bioreactor. In this text, the term “batch fermentation” in particular denotes a fermentation process, wherein there is no removal of a culture suspension cultivated in the bioreactor with the exception of insignificant amounts required for analytical testing, and wherein there is no addition of fresh dispersing or cultivation medium into the bioreactor. Furthermore, a flow of gaseous compounds into and out of the bioreactor during the fermentation process, such as inflow of inert gas to maintain anaerobic cultivating conditions or such as outflow of metabolic exhaust gas, are not considered as material added or removed from the bioreactor. Thus, in this text the term “batch fermentation” with respect to addition and removal of gaseous compounds does not denote a process in a closed system.

The term “fed-batch fermentation” is known and denotes a fermentation process in a bioreactor, wherein during the fermentation process no material, in particular no-culture suspension is removed from the bioreactor, except for insignificant amounts required for analytical testing and except for gaseous compounds. However, in a fed-batch fermentation process, material is added to the bioreactor during the fermentation process, in particular fresh dispersing medium is added. The added dispersing medium may be the same or different dispersing medium as the dispersing medium in the bioreactor at the beginning of the fed-batch fermentation process.

Batch cultivation such as in an anaerobic batch or fed-batch fermentation process in the field of biotechnology is known to be particularly suitable for large-scale production of microbes such as bacteria. The terms “continuous culture”, “continuous cultivation” and “continuous co-cultivation” are known and refer to a cultivation of microbes, in particular bacterial strains, in a bioreactor comprising a liquid dispersing or culture medium wherein during the cultivation process materials are added and removed. In particular, the term “continuous culture” refers to a cultivation process wherein fresh medium replaces an equal volume of effluent of culture-suspension at a constant flow rate during the cultivation process. The terms “dispersing medium”, “cultivation medium” and “culture medium” are used interchangeably herein and refer to a liquid or solid medium in which the bacterial strains are inoculated and/or cultivated. As used herein, the term “bioreactor” refers to a device or apparatus in which a biological reaction or process is carried out, especially on an industrial scale.

The term biotechnological production of an in vitro assembled consortium of bacterial strains on a large scale or similarly on an industrial scale in particular denotes volumes of the culture-suspension during anaerobic fermentation above laboratory scale, i.e. in particular above 200 ml, in particular above 300 ml or 500 ml and in particular refers to volumes of the culture-suspension during anaerobic batch cultivation of at least 1 It, 10 It, 30 It, 100 It or 500 It.

The term “at least one” means “one or more”. For instance, it refers to one, two, three or more.

Consortium

The present invention provides a process for producing a defined consortium as a final product in a reproducible way and with high yield, compatible with industrial scale requirement. It is based on rules to design the consortium and on a particular process for preparing a preserved inoculum. Based on this preserved inoculum, the defined consortium can be prepared as a final product by batch fermentation. More specifically, the advantages of the method according to the present invention include a simple and robust production, increased production of the final product with better preservation of the desired functionalities, higher survival of single strains, increased resistance to stress applied during downstream processing and robust reproducibility of the targeted composition. More particularly, starting from the inoculum of the present invention, shorter lag phase and faster growth of all bacteria of the consortium have been observed after inoculation.

The present invention provides in particular a method of manufacturing an in vitro assembled consortium by an anaerobic co-cultivation in a dispersing or culture medium. In the context of the invention, the co-cultivation process relies on the incubation of different bacterial strains that have been selected based on their metabolic functions, particularly to establish a trophic network in which bacteria collaborate. Then, the consortium comprises at least three different bacteria or a plurality of functional groups. Each functional group comprises at least one bacterium of the selected bacterial strains. Each functional group performs at least one metabolic pathway of an anaerobic microbiome, in particular of an intestinal microbiome, or another anaerobic microbiome such as for example a buccal microbiome, a vaginal microbiome, a skin microbiome, waste-treatment microbiome, soil microbiome, a plant-associated microbiome, a microbiome used for anaerobic food fermentation. Preferably, the consortium comprises at least three bacterial strains (i.e. at least three different bacterial strains). Each of the bacterial strain of the consortium belongs to at least one of the functional groups.

The method of manufacturing the in vitro assembled consortium comprises the steps of:
I. providing a sample of the assembled consortium as an inoculum; more specifically, the sample of the consortium is obtained from a prior continuous anaerobic co-cultivation process of the selected bacterial strains at least until a stable microbial profile and a stable metabolic profile is obtained and the sample being preferably obtained as a preserved sample;
II. adding the inoculum to the dispersing medium in a bioreactor thereby forming a culture-suspension of the selected bacterial strains;
III. multiplying the selected bacterial strains in the culture suspension by co-cultivation until a stable microbial profile and a stable metabolic profile characteristic of the in vitro assembled consortium is established; more specifically, step III is performed in an anaerobic batch fermentation process or in an anaerobic fed-batch fermentation process; and
IV. harvesting the consortium of the selected live, viable bacterial strains;
V. optionally, subjecting the harvested consortium to one or more post-treatment steps.

The term “post treatment” preferably refers to a further processing step or downstream treatment, such as for example a preservation treatment.

Thus, advantageously and surprisingly, the present invention provides methods of in vitro assembled consortia with a stable microbial profile and in particular also with a stable metabolic profile during anaerobic co-cultivation as well as methods of manufacturing them on a large scale by an anaerobic batch co-cultivation, despite variable substrate affinities and growth rates of the bacterial strains present in the in vitro assembled consortia. Method of manufacturing are more particularly disclosed here below under the paragraph “Method of manufacturing”.

Functional Groups and Metabolic Pathways

Contrary to what is generally envisioned in the microbiome field, which is to replace a particular dysfunctional or missing bacterium by another, the inventors focused on the functions performed by bacteria in the intestinal microbiome. Then, the consortium disclosed herein is not particularly defined by a particular composition of specific bacteria but by a combination of functions or capacities fulfilled by bacteria to allow their interaction or collaboration, their maintenance in the consortium and/or the production of particular metabolites. Fiber and protein degradation by bacterial fermentation in the intestine is the central function of the intestinal microbiome (Chassard and Lacroix 2013). It is generally known that intestinal fermentation is performed through close interactions between functional groups of which the most important are illustrated in FIG. 1.

Capacities of bacteria to degrade or convert a particular substrate (e.g. starch) and to produce a particular product or metabolite (e.g. butyrate) rely on metabolic pathways. Then, a functional group comprises bacteria that are able to degrade or convert the same substrate(s) (e.g. starch) and to produce the same metabolite(s) (e.g. butyrate), i.e. bacteria that are able to perform similar metabolic pathways. Such functions or capacities of a bacterium are well known in the art. For example, experiments are known to test if a bacterial strain is able to perform a metabolic pathway and thus belongs to a particular functional group. For example, the degradation of sugars, starches or fibers can be tested simply by providing such substrate to bacteria while observing or monitoring their growth. For example, bacteria can be characterized for growth and metabolite production on M2GSC Medium (ATCC Medium 2857) and modifications thereof whereby the carbon sources glucose, cellobiose and starch are replaced by specific substrates including intermediate metabolites and/or fibers, preferably such as found in the human intestine. The concentrations of the produced metabolites can for example be quantified by any analytic method available for the person skilled in the art such as refractive index detection high pressure liquid chromatography (HPLC-RI; for example, as provided by Thermo Scientific Accela™).

In one embodiment, the consortium of the invention is defined by metabolic pathways that are performed by bacterial strains. Preferably such metabolic pathways are based on the degradation or conversion of a substrate, an intermediate metabolite or an end metabolite; and on the production of an intermediate metabolite or an end metabolite.

For example, pathway 1 (P1) corresponds to the conversion of sugars, starches, fibers or proteins and the production of formate

Pathway 2 (P2) corresponds to the conversion of sugars, starches, fibers or proteins and to the production of acetate.

Pathway 3 (P3) corresponds to the conversion of sugars, starches, fibers or proteins to the production of butyrate.

Pathway 4 (P4) corresponds to the conversion of sugars, starches, fibers or proteins and to the production of lactate.

Pathway 5 (P5) corresponds to the conversion of sugars, starches, fibers or proteins and to the production of succinate.

Pathway 6 (P6) corresponds to the conversion of formate and to the production of acetate.

Pathway 7 (P7) corresponds to the conversion of acetate and to the production of butyrate.

Pathway 8 (P8) corresponds to the conversion of lactate and to the production of butyrate.

Pathway 9 (P9) corresponds to the conversion of lactate and to the production of propionate.

Pathway 10 (P10) corresponds to the conversion of succinate and to the production of propionate.

Pathway 11 (P11) corresponds to the conversion of sugars, starches, fibers or proteins, to the reduction of oxygen and to the production of lactate.

Pathway 12 (P12) corresponds to the conversion of hydrogen, carbon dioxide or formate and to the production of acetate.

Pathway 13 (P13) corresponds to the conversion of peptides and to the production of propionate.

Then, the consortium of the invention comprises a set of bacterial strains, the set being able to perform a plurality of pathways, preferably at least three different metabolic pathways selected from the group consisting of P1, P2, P3, P4, P5, P6, P7, P8, P9, P10, P11, P12 and P13 as defined above.

In a particular aspect, each of the bacterial strains of the consortium is able to perform at least two metabolic pathways but no more than five metabolic pathways. Preferably, each of the bacterial strains of the consortium performs no more than 4, 5, 6 or 7 pathways at the same time. This means that a particular bacterial strain is not able to perform all of the 13 pathways (P1-P13) as described above. For example, bacteria such as Faecalibacterium prausnitzii, are able to perform pathways 1, 2, 3 and 7. For instance, Table 1 provides information regarding bacterial strains, metabolic pathways and functional groups.

TABLE 1

| | Function | | |
|---|---|---|---|
| | (included functional pathway | Metabolic | |
| | number in brackets refer to FIG. | Pathway | Functional |
| Bacterial strain | 1) | (FIG. 1) | Group |
| Ruminococcus bromii | Resistant starch degrader and | 1, 2 | A1 |
| Eubacterium eligens | formate and/or acetate producer (1, | | |
| | 2) | | |
| Faecalibacterium prausnitzii | Starch degrader (1), acetate- | 1, 2, 3, 7 | A2 |
| Roseburia intestinalis | consuming and butyrate-producer | | |
| | (3, 7) | | |
| Lactobacillus rhamnosus | O₂ reducer (11), lactate (4) and | 1, 4, 11 | A3 |
| Enterococcus faecalis | formate producer (1) | | |
| Bifidobacterium adolescentis | Starch degrader, lactate (4), formate | 1, 2, 4 | A4 = A7 |
| Roseburia hominis | (1) and acetate producer (2) | | |
| Anaerotignum (former | Protein degrader (3), lactate- | 3, 9 | A5 |
| Clostridium) lactatifermentans | utilizing and propionate producer | | |
| Coprococcus catus | (9) | | |
| Eubacterium limosum | Starch degrader (2), lactate | 2, 8 | A6 |
| Eubacterium hallii | degrading and acetate and butyrate | | |
| | producer (8) | | |
| Collinsella aerofaciens | Starch degrader, lactate (4), formate | 1, 2, 4 | A7 = A4 |
| Roseburia hominis | (1) and acetate producer (2) | | |
| Phascolarctobacterium faecium | Protein degrader (13), succinate- | 10, 13 | A8 |
| Flavonifractor plautii | reducing, propionate producer (10) | | |
| Blautia hydrogenotrophica | Functional pathway: H₂ reducer | 6, 12 | A9 |
| | (12), formate-reducing acetate- | | |
| | producer (6) | | |
| Bacteroides xylanisolvens | Starch degrader (2), succinate (5) | 2, 4, 5, (“14”) | A10/A11 |
| | and propionate producer (4) GABA | | |
| | producer (“14”) | | |
| Bacteroides fragilis | fiber degrader (2), succinate (5) | 2, 5, 10, 13 | A10 |
| | and propionate producer (10, 13) | | |
| Clostridium scindens | Conversion from primary to | 15 | A12 |
| | secondary bile acids (“15”) | | |
| Eubacterium limosum | A6: Starch degrader (2), lactate | 2, 6, 8, 12 | A6/A9 |
| | degrading, acetate and butyrate | | |
| | producer (8) | | |
| | A9: H₂ reducer (12), formate- | | |
| | reducing, acetate-producer (6) | | |

Ability of bacterial strains to perform particular metabolic pathways allows the definition of functional groups. This means that the composition of the consortium may be not only defined by its capacity to perform particular metabolic pathways, but also by the repartition of bacterial strains into functional groups. For example, bacterial strains such as Faecalibacterium prausnitzii, are able to perform pathways 1, 2, 3 and 7 and thus may be classified into functional group A2.

In one embodiment, the functional groups according to the invention are defined as follows:
(A1) Resistant starch degraders utilizing one or more of the pathways 1,2;
(A2) Starch degrading-, acetate-consuming butyrate-producers utilizing one or more of the pathways 1, 3, 4, 7;
(A3) Oxygen-reducing lactate- and formate-producers utilizing one or more of the pathways 1, 4, 11;
(A4) Starch-reducing lactate- and formate-producers utilizing one or more of the pathways 1, 2, 4;
(A5) Protein- and lactate-utilizing propionate-producers utilizing one or more of the pathways 13, 9;
(A6) Starch-, protein- and lactate-utilizing butyrate-producers utilizing one or more of the pathways 3, 8;
(A7) Starch- and protein-degrading formate- and lactate-producers utilizing one or more of the pathways 1, 2, 4;
(A8) Protein-, succinate-utilizing, propionate-producers utilizing one or more of the pathways 10;
(A9) Hydrogen- and formate-utilizing acetate-producers utilizing one or more of the pathways 6,12;
(A10) is an additional functional group of succinate producers utilizing the pathway 5, wherein the pathways 1-13 are key metabolic pathways of an intestinal microbiome, as defined in figure.
(A11) is an additional functional group of Protein—utilizing and acetate and butyrate producers;
(A12) is an additional functional group of proteins, fibers, starches or sugars consumers and biogenic amines producers such as y-aminobutyric acid (GABA), cadaverin, dopamine, histamine, putrescine, serotonin, spermidine and/or tryptamine producers;
(A13) is an additional functional group of primary bile acids consumers and secondary metabolites producers;
(A14) is an additional functional group of vitamins producers such as cobalamin (B12), folate (B9) or riboflavin (B2);
(A15) is an additional functional group of mucus degraders.

Preferably, the functional groups according to the invention are defined as follows:
Bacterial strains of functional group (A1) have the capacity of consuming sugars, fibers and resistant starch and producing formate and acetate. Preferably, bacteria of functional group A1 perform the metabolic pathways 1 and 2.
Bacterial strains of functional group (A2) have the capacity of consuming sugars, starch and acetate and producing butyrate and formate. Preferably, bacteria of functional group A2 perform the metabolic pathway 7, preferably in combination with pathways 2 and/or 3, optionally with pathways 1, 2 and 3.
Bacterial strains of functional group (A3) have the capacity to degrade sugars, and to reduce oxygen, and to produce lactate, optionally formate. Preferably, bacteria of functional group A3 are able to perform pathways 1, 4 and 11;
Bacterial strains of functional group (A4) degrade sugars, starches, fibers or protein and carbon dioxide and produce lactate and/or formate, optionally acetate. Preferably, bacteria of functional group A4 perform the metabolic pathways 1, 2 and 4, optionally 2;
Bacterial strains of functional group (A5) degrade protein and/or lactate and produce propionate and optionally acetate. Preferably, bacteria of functional group A5 perform the metabolic pathways 9 and 13;
Bacterial strains of functional group (A6) degrade starches, protein and lactate and produce butyrate and hydrogen, optionally acetate. Preferably, bacteria of functional group A6 perform the metabolic pathways 2, 3 and 8;
Bacterial strains of functional group (A7) degrade sugars, starches and optionally formate, and produce formate, lactate and optionally acetate. Preferably, bacteria of functional group A7 perform the metabolic pathways 1, 2 and 4;
Bacterial strains of functional group (A8) degrade protein and succinate and produce propionate and acetate. Preferably, bacteria of functional group A8 perform the metabolic pathway 10 and 13;
Bacterial strains of functional group (A9) degrade sugars, fibers, protein, carbon dioxide, hydrogen and/or formate and produce acetate. Preferably, bacteria of functional group A9 perform the metabolic pathways 2, 6 and/or 12;
Bacterial strains of functional group (A10), which is an additional or optional functional group, comprises bacteria consuming sugars, fibers, and resistant starch, and producing succinate, preferably performing the metabolic pathway 5.
Bacterial strains of functional group (A11), which is another additional or optional functional group, comprises bacterial strains consuming proteins and producing acetate and/or butyrate. Preferably, bacteria of functional group A11 perform the metabolic pathways 2 and 3.
Bacterial strains of functional group (A12) have the capacity of consuming proteins, fibers, starches or sugars, and producing biogenic amines such as y-aminobutyric acid (GABA), cadaverine, dopamine, histamine, putrescine, serotonin, spermidine and/or tryptamine.
Bacterial strains of functional group (A13) have the capacity of consuming primary bile acids and producing secondary metabolites.
Bacterial strains of functional group (A14) have the capacity of producing vitamins such as cobalamin (B12), folate (B9) or riboflavin (B2).
Bacterial strains of functional group (A15) have the capacity of consuming mucus.

In a particular aspect, each of the bacteria of the consortium belongs to at least one functional group but to no more than 2, 3, 4 or 5 functional groups. This means that a particular bacterial strain cannot belong to all of the 10 functional groups (A1-A10) as described above. In another particular embodiment, each of the functional groups comprises only one bacterial strain. In another particular embodiment, the functional groups comprise more than one bacterial strain.

Consortium Assembly

A way to assemble a consortium is based on the following rationale for the selection of suitable bacterial strains to be assembled into a plurality that is capable of establishing a stable consortium during anaerobic co-cultivation:

1. An in vitro assembled consortium mirrors selected parts of a corresponding physiological microbiome, in particular of the intestinal microbiome. A microbiome is a trophic network of microorganisms, in particular bacteria, with different affinities to substrates such as the selected nutrients and different growth-rates on the respective substrates. For bacterial strains in the human intestinal microbiome, for example, the substrates can be of dietary origin, produced by the host or produced by other bacteria in the microbiome.

2. The stabilization of the composition of the microbiome over time, i.e. the relative abundances of microbes and thus metabolic functions and amounts of metabolites, is based on the establishment of a trophic network based on continuous cross-feeding allowing availability of substrates, including in particular, intermediate metabolites as substrates at growth promoting concentrations. For example, a cross-feeding interaction or collaboration between bacteria could be: bacterium 1 degrades or converts a particular substrate (e.g. starch) and produces a particular intermediate metabolite (e.g. formate) that is used as a substrate by bacterium 2 to produce an end metabolite (e.g. acetate). Such a trophic network includes cross-feeding between microbial, in particular bacterial, strains, and includes a synchronisation of the different strains through interactions while performing the various metabolic functions under avoidance of accumulation of inhibitory concentrations of intermediate metabolites (Chassard & Lacroix, 2013). This synchronization of growth and production of the respective metabolites allows the maintenance of each of the bacterial strains at a favourable growth rate due to availability of substrate and prevention of accumulation of inhibitory concentrations of metabolites into the consortium and the production of defined end metabolites. Bacterial strains sharing a majority of metabolic function(s) are referred to as a functional group, i.e. bacteria performing similar metabolic pathways belong to the same functional group. Then, the bacteria of the consortium are selected so as to obtain the desired end metabolites and to avoid inhibitory concentration of intermediate metabolites and by-products through the design of a trophic network.

FIG. 1 shows a schematic illustration of primary pathways of substrate, i.e. nutrient, degradation, cross-feeding pathways, and inhibitory pathways occurring in the intestinal microbiome.

“Primary pathways” are pathways in which substrates (nutrients) are converted to intermediate metabolites or end metabolites. For instance, it could be pathways 1-5 and 13 as discussed above and described in FIG. 1.

“Cross-feeding pathways” are pathways in which intermediate metabolites or end metabolites produced by some bacterial strains of the consortium are converted to end metabolites by other bacterial strains of the consortium. For instance, it could be pathways 6-10 as discussed above and described in FIG. 1. “Inhibitory pathways” are pathways wherein some bacterial strains of the consortium can produce inhibitory concentrations of a compound such as a metabolite. For instance, it could be one or more of pathways 1, 4, 5, 11 or 12 as discussed above and described in FIG. 1.

Such an accumulation of an inhibitory compound prevents the reproduction of an identical in vitro assembled consortium by co-cultivation. Indeed, the presence of an intermediate metabolite or by-product in an inhibitory concentration may destabilize the assembled consortium and/or lead to toxicity upon administration of the consortium to a subject. If one functional group is eliminated from the plurality of functional groups of the assembled consortium, for example due to inhibitory concentration, this will lead to the destabilization of the consortium, i.e. alteration of the metabolic and microbial profiles of the consortium. Inhibition of proliferation of only a single one of the selected bacterial strains may result in elimination of a functional group and to the complete destabilization of the consortium. Similarly, inhibition of all of the selected strains of a particular functional group may result in its elimination from the consortium. It is thus mandatory to select bacteria, pathways and functional groups that taken together allow the establishment of a stable consortium, i.e. a consortium that equilibrates at a defined composition based on the cross-feeding and absence of mutual inhibition. For example, FIG. 1 indicates ten functional groups of bacteria, defined as A1-A10, performing the above-mentioned pathways of the intestinal microbiome.

3. During anaerobic co-cultivation, the plurality of selected bacterial strains fulfils particular criteria, preferably criteria (a) and (b). More particularly, the plurality of selected bacterial strains is able to produce at least one end metabolite and comprises: at least one bacterial strain which produces an intermediate metabolite and at least one bacterial strain which converts the intermediate metabolite, preferably into an end metabolite. The plurality of selected bacterial strains produces metabolites and creates local gradients with respect to substrate concentration, pH and Redox potential. Accordingly, such a plurality of selected bacterial strains produces at least one end metabolite while avoiding intermediate metabolites accumulation. These gradients establish and maintain niches for growth of particular functional groups and selected bacterial strains. This stabilizes an in vitro assembled consortium. Such niche phenomena are known not only from the physiological environment in the intestine but also observed in in vitro, e.g. as published for the anaerobe bacteria Faecalibacterium prausnitzii (Khan et al., 2012). FIG. 1 and the description below details selected metabolic interactions and functional groups of the intestinal microbiome.

A consortium according to the present invention could be defined as follows:
the consortium comprises at least three bacterial strains;
each bacterial strain of the consortium performs at least one metabolic pathway of an anaerobic trophic network, in particular an intestinal microbiome;
in said trophic network, the consortium performs a conversion of substrate into end metabolite, preferably into a short chain fatty acid, even more preferably selected from acetate, propionate and butyrate; and
the bacterial strains of the consortium are selected to enable metabolic cross-feeding interactions or collaboration between each other during co-cultivation, so as the consortium comprises at least one first bacterium being able to produce a metabolite and at least one second bacterium which converts said metabolite.

Preferably, to enable metabolic cross-feeding interactions or collaboration, the metabolite is an intermediate metabolite. For instance, said intermediate metabolite can be selected from formate, lactate and succinate. Preferably, the bacterium which converts the intermediate metabolite produces an end metabolite. Alternatively or in addition, the bacterium which converts the metabolite converts an end metabolite into another end metabolite.

Accordingly, in the trophic network, the conversion or degradation of a substrate can be performed directly or indirectly through an intermediate metabolite. More specifically, the conversion may be performed at least partially indirectly through an intermediate metabolite. Then, the conversion into an end metabolite can be performed directly from the substrate and also indirectly through an intermediate metabolite. In addition or alternatively, the conversion into an end metabolite can be performed directly from the substrate and also indirectly through another end metabolite.

Preferably, the consortium and/or the method is designed so as to fulfil at least one of the criteria below, in particular during the step 11:
According to criteria (a), the bacterial strains together perform a degradation or conversion of a substrate into an end metabolite. In one embodiment, the end metabolite can be a short chain fatty acid, even more preferably be selected from acetate, propionate and butyrate and mixtures thereof. Then, in this embodiment, the selected bacterial strains together perform a degradation of the selected nutrients directly, or indirectly via an intermediate metabolite, to a short chain fatty acid, in particular to one or more of acetate, propionate and butyrate.
According to criteria (b), the bacterial strains are selected to enable metabolic cross-feeding interactions or collaboration between each other during co-cultivation, so as the bacterial strains comprise at least one first bacterium being able to produce an intermediate metabolite and at least one second bacterium which converts said intermediate metabolite. In one embodiment, said intermediate metabolite is selected from formate, lactate and succinate and mixtures thereof. In this embodiment, the bacterial strains comprise a functional group or a bacterium which produces a particular intermediate metabolite and a functional group a bacterium consuming said intermediate metabolite, preferably said intermediate metabolite being selected from formate, lactate and succinate.
According to criteria (c), the bacterial strains are selected to maintain concentrations of intermediate metabolites in the culture medium below a concentration inhibiting proliferation of at least one bacterial strain of the consortium. In one embodiment, the intermediate metabolite is selected from formate, lactate and succinate and mixtures thereof. Preferably, the concentration in the medium or culture-suspension of any intermediate metabolite produced during the degradation is below the concentration inhibiting proliferation of all bacterial strains provided in one of the functional groups.
According to criteria (d), the bacterial strains are selected to maintain concentrations in the culture medium of inhibitory by-products of the trophic network below a concentration inhibiting proliferation of at least one bacterial strain of the consortium. For instance, the inhibitory by-products can be selected from hydrogen and oxygen and mixtures thereof. More specifically, a concentration in the culture medium or culture-suspension of one or more inhibitory compounds produced as a by-product of the degradation, in particular H2, or a concentration in the culture-suspension of environmental or dissolved O2, is below the concentration inhibiting proliferation of all bacterial strains provided in one of the functional groups.

Preferably, the consortium according to the invention fulfils criteria (a) and (b). In some embodiments the consortium according to the invention fulfils criteria (a), (b) and (c). In some embodiments the consortium according to the invention fulfils criteria (a), (b) and (d). Preferably, the consortium according to the invention fulfils criteria (a), (b) (c) and (d).

It is important that one or more of the criteria (a), (b) (c), (d) are fulfilled by the consortium during the step of production of the final product by the anaerobic batch or fed batch co-cultivation (step Ill), especially criteria (a) and (b).

Exemplary compositions of in vitro assembled consortia comprise some or all of the functional groups A1-A10 as illustrated in FIG. 1. The functional groups A1 to A10 or A1 to A11 are chosen to provide metabolic interactions capable of promoting optimal growth and establishment of an equilibrium if the plurality of selected strains comprises functional groups capable of fulfilling one or more than one of the criteria (a), (b), (c), (d) during the anaerobic batch co-cultivation of step Ill.

As shown in FIG. 1, all primary substrates are degraded through pathways 1-5 present in the functional groups A1-A10. Degradation of primary substrates can result in formation of intermediate metabolites, in particular formate, lactate, succinate and gases like hydrogen. As noted above accumulation of intermediate metabolites or gases to high levels can be inhibitory and bacterial growth for the production of beneficial end-metabolites. Functional groups performing pathways 6 to 13 may therefore be vital for establishment of an equilibrium between production and consumption of intermediary metabolites to keep their concentrations below an inhibitory level and thereby enabling growth.

Based on the above outlined rationale, the consortia provided as inoculum in step I of the method are assembled in vitro from isolated bacterial strains. The exemplary consortium PB002 used as an exemplary inoculum in step I is described in WO2018189284, the content thereof being incorporated by reference, comprises the plurality of functional groups A1 to A9.

It has been observed that furthermore, consortia comprising subsets of functional groups of A1 to A9 or comprising the additional functional A10 assembled according to the rationale described above surprisingly also stabilize during anaerobic co-cultivation with a characteristic stable microbial and stable metabolic profile. Thus, advantageously, a collection of various in vitro assembled consortia may be designed according to the rationale described above, all of which can be produced by anaerobic co-cultivation in the method of the present invention.

The in vitro assembled consortia that are manufactured by the method of the present invention may comprise some or all of the exemplary functional groups of bacterial strains (A1) to (A10) shown in FIG. 1 or some or all of the exemplary functional groups of bacterial strains (A1) to (A11). Alternatively, the in vitro assembled consortia that are manufactured by the method of the present invention may comprise live, viable bacteria that are able to perform some or all of the metabolic pathways (P1) to (P13) as shown in FIG. 1. In some embodiments of the method of manufacturing in vitro assembled consortia, the plurality of functional groups is selected from functional groups of bacterial strains that are present in the intestinal microbiome, such as the exemplary functional groups (A1) to (A10) are represented by intestinal bacterial strains.

In some embodiments of the methods of manufacturing or providing in vitro assembled consortia designed to mirror parts of the intestinal microbiome, the consortium may include a selected bacterial strain that is not a physiological intestinal bacterial strain or at least not known to be a physiological intestinal bacterial strain.

In pure culture, the functions of single bacterial strains of the functional groups may be bidirectional. For example, (A7) may either produce or consume formate. However, when combined in the in vitro assembled consortia, the bacterial strains show the properties discussed herein, degrading the selected nutrients directly, or indirectly via an intermediate metabolite, to a short chain fatty acid, in particular to one or more of acetate, propionate and butyrate, consuming intermediate metabolites (succinate, lactate, formate).

In one embodiment, the end metabolites are predominantly produced meaning that intermediate metabolites are not found in higher concentrations than 15 mM each. Preferably, intermediate metabolites such as formate, lactate and succinate are not found in higher concentrations than 15 mM each.

The in vitro assembled consortia may also be described as synthetic and symbiotic consortia which are characterized by a combination of microbial activities forming a trophic chain from complex fiber metabolism to the canonical final SCFAs (Short chain fatty acids) found in the healthy intestine: acetate, propionate and butyrate. This trophic completeness prevents the accumulation of potentially toxic or pain inducing products such as H2, lactate, formate and succinate. Activities are screened by functional characterization on different substrates of the human gut microbiota. However, type and origin of strains can be selected according to the targeted level of complexity of the in vitro assembled consortia in order to recompose a consortium combining the desired functional groups. The exemplary consortia PB002, PB003, PB004, PB010 and PB011 ensure degradation of complex polysaccharides usually found in the gut (resistant starch, xylan, arabinoxylan, cellulose and pectin), reutilization of sugars released, removal of environmental O2 traces for maintenance of anaerobiosis essential for growth, production of key intermediate metabolites and gases (acetate, lactate, formate, and H2), reutilization of all intermediate metabolites and production of end metabolites found in a healthy gut (acetate, propionate and butyrate).

The in vitro assembled consortia exclusively produce the desired metabolites in defined ratios that are targeted for therapeutic use supporting the production of beneficial metabolites used by the host for different functions such as acetate (energy source for heart and brain cells), propionate (metabolized by the liver) and butyrate (the main source of energy for intestinal epithelial cells).

The exemplary in vitro assembled consortium PB002 comprises groups providing for the following functions:

Degrade the main energy sources in the gut including fibers and intermediate metabolites (all groups)

Protect anaerobiosis by reduction of the eventual 02 through respiration (A3);

Produce the main end metabolites found in the intestine (A1, A2, A3, A4, A5, A9);

Prevent the enrichment of intermediate metabolites (A5, A6, A7, A8, A9).

The exemplary in vitro assembled consortium PB010 and PB011 comprise groups providing similar functions (i.e. all functions A1 to A9 are present) but includes different compositions of bacteria, in terms of number of strains or of genera involved. This shows the modularity of the assembled consortium and underlines the robustness of assembly based on functions rather than on specific bacterial strain. PB011 show the possibility to extend the assembly to further functional groups such as functional group A10 This combination of functional groups of bacteria (A1) to (A9), encompass the key functions of fiber degradation by the microbiome as described by Lacroix and Chassard in 2013 and results, if cultured together, in a trophic chain or network analogue to the healthy intestinal microbiome in its capacity to exclusively produce end metabolites from complex carbohydrates without accumulation of intermediate metabolites, particularly in inhibitory concentration. It is particularly beneficial that the combination of strains from the functional groups (A1) to (A9) prevents the enrichment of intermediate metabolites independent of the composition of the recipient's microbiome and the relative concentration of the enriched intermediate metabolites. This is why the consortium disclosed herein is not particularly defined by a specific composition of bacterial strains but by a combination of particular functions, e.g. A1 to A9, optionally A1 to A10 or A1 to A11.

Then, a further aspect of the invention concerns a method of providing an in vitro assembled consortium of selected live, viable bacterial strains. The consortium of selected live, viable bacterial strains comprises a plurality of functional groups comprising a subset of functional groups A1 to A9. Preferably, the consortium of selected live, viable bacterial strains comprises at least two or at least three different functional groups selected from the group consisting of A1, A2, A3, A4, A5, A6, A7, A8 and A9. Alternatively, the consortium comprises a plurality of functional groups comprising A1 to A10 or subsets thereof. Preferably, the consortium of selected live, viable bacterial strains comprises at least three different functional groups selected from the group consisting of A1, A2, A3, A4, A5, A6, A7, A8, A9 and A10. Functional groups A1 to A10 are indicated FIG. 1 and further described in more detail in this text. It is understood that a consortium that is assembled in vitro according to this aspect of the invention may serve as inoculum in the method of manufacturing an in vitro assembled consortium of selected live, viable bacterial strains by an anaerobic co-cultivation, particularly in step I of the method of manufacturing or in step (a) of a preparatory stage of the method, such as disclosed here below under the paragraph “Method of Manufacturing”. Optionally, the consortium comprises a plurality of functional groups comprising A1 to A11 or subsets thereof, for instance at least three different functional groups selected from the group consisting of A1, A2, A3, A4, A5, A6, A7, A8, A9, A10 and A11.

Alternatively, the consortium comprises selected live, viable bacterial strains able to perform a plurality of metabolic pathways P1 to P13 or subsets thereof. Preferably, the consortium comprises selected live, viable bacterial strains able to perform at least two different metabolic pathways selected from the group consisting of P1, P2, P3, P4, P5, P6, P7, P8, P9, P10, P11, P12 and P13 and any subsets thereof.

A list of exemplary methods of providing the in vitro assembled consortium comprising a subset of functional groups A1 to A9 or comprising functional groups A1 to A10 or subsets thereof is presented below:
If formate is produced by functional group A3, A4 or A7, for instance through pathway 1, formate has to be removed to prevent its inhibitory effect on the bacterial growth and production of end metabolites.
Formate is removed by group A9, through pathway 6 or pathways 6 and 12.
If pathway 12 is used to remove formate, H2 has to be produced, by a hydrogen producing group such as A2 through pathway 3, or group A6 through pathway 8.
If lactate is produced through pathway 4, for instance through the functional group A3, A4 or A7, lactate has to be removed to prevent its inhibitory effect on the bacterial growth and production of end metabolites.
Lactate is removed by group A5 producing propionate or by group A6 producing butyrate.
Butyrate production by Group A6 can produce self-inhibiting hydrogen that can be removed by group A9, using pathway 12.
If succinate is produced through pathway 5, by the functional group A10, succinate has to be removed to prevent its inhibitory effect on the bacterial growth and production of end metabolites.
Succinate is removed by group A8 producing propionate.
If oxygen is present it has to be removed to prevent its inhibitory effect on the bacterial growth and production of end metabolites.
Oxygen can be removed through pathway 11 by group A3.
If hydrogen is present, it has to be removed to prevent its inhibitory effect on the bacterial growth and production of end metabolites.
Hydrogen can be removed through pathway 12 by group A9.
Functional group A9 requires the presence of formate, that is produced by the functional groups A3, A4 and A7.
If acetate is present, it can be converted to the beneficial end metabolite butyrate by the functional group A2.

In yet a further aspect of the invention, a composition is provided comprising an in vitro assembled consortium of selected live, viable bacterial strains, obtainable by the method of providing an in vitro assembled consortium described above.

In some embodiments, the methods of the present invention and the composition of the present invention as described herein, the plurality of functional groups is selected to fulfil both criteria (a) and (b) as defined above in the context of step III of the method of manufacturing the in vitro assembled consortium.

The functional groups—or groups for short—(A1) to (A10) are described in more detail above: Their metabolic functions and exemplary strains as listed. However, it is understood that only a subset of these functional groups or additional functional groups of bacteria may be present in the in vitro assembled consortia described herein. Variable assemblies of functional groups may e.g. further improve or alter therapeutic applications or may have a beneficial effect on the production process or preservation methods for the consortia.

The following exemplary embodiments of pluralities of pathways result in consortia that fulfil criteria (a) and (b):
pathway P1 in combination with pathway P6, optionally with pathway P2 and pathway P7;
pathway P4 in combination pathway P8 and/or pathway P9, optionally with pathway P3, pathway P13;
pathway P5 in combination with pathway P10, optionally pathway P13;
pathway P5 in combination with pathway P10, pathway P13, pathway P4 and pathway P9;
pathway P4 in combination with pathway P8, pathway P3, optionally with pathway P2 and pathway P7.

Bacteria performing Pathway P11 can be added to any of these combinations in order to remove oxygen whereas bacteria performing pathway P12 can be added to remove hydrogen.

Any of these combinations can be used in the method according to the present invention.

In addition, the following exemplary embodiments of pluralities of functional groups of bacteria result in consortia that fulfil criteria (a) and (b):
A6 in combination with A3, A4 or A7, whereby A6 produces butyrate from lactate produced by A3, A4 or A7 through degradation of primary substrates. This combination can further comprise A9 in order to produce acetate from the formate produced by A4 and/or A7 if present.

Further this combination can further comprise A9 in order to produce acetate from the hydrogen produced by A6 through the production of butyrate using P8.
A5 in combination with A3, A4 or A7, whereby A5 produces propionate from lactate produced by A3, A4 or A7 through degradation of primary substrates. This combination can further comprise A9 in order to produce acetate from the formate produced by A4 and/or A7 if present.
A5 and A6 in combination with A3, A4 or A7, whereby A5 produces propionate from lactate, A6 produces butyrate from lactate and lactate is produced by A3, A4 or A7 through degradation of primary substrates.

This combination can further comprise A9 in order to produce acetate from the formate produced by A4 and/or A7 if present.

This combination can further comprise A9 in order to produce acetate from the hydrogen produced by A6 through the production of butyrate using P8.
A9 in combination with A3, A4 or A7, whereby A9 produces acetate from formate produced exclusively by A3, A4 or A7 through degradation of primary substrates.

This combination can further comprise A6 in order to produce butyrate from the lactate produced by A3, A4 or A7.
A6 in combination with A3, A4 or A7 and A9, whereby A6 produces butyrate from lactate produced from primary substrates by A3, A4 or A7 and A9 produces acetate through formate produced from primary substrates by A3, A4 or A7 and hydrogen produced by A6.
A5 in combination with A3, A4 or A7 and A9, whereby A5 produces propionate from lactate produced from primary substrates by A3, A4 or A7 and A9 produces acetate through formate produced from primary substrates by A3, A4 or A7.
A9 in combination with A1, A2 or A4, whereby A9 produces acetate from formate produced by A1, A2 or A4 through degradation of primary substrates. A5 and/or A6 can be added in the combination in order to convert lactate if A4 is present.
A10 and A8, whereby A8 produces propionate through succinate from primary substrates by A10.
A1 and A2, whereby A2 produces butyrate from acetate produced by A1 from primary substrates.

In addition, when necessary, additional groups can be added in order to remove inhibitory by-products such as hydrogen or oxygen, for instance group A3 for oxygen and group A9 for hydrogen.

Further consortia combining the above modules or subnetworks and combining multiple bacterial strains for each functional group fulfil criteria a and b of step Ill, too. Then, the assembly of modules or subnetworks as defined hereabove allows to create different consortia that fulfil at least the criteria (a) and (b). This shows the modularity of the consortium of the invention and the rationale to build a stable consortium.

Preferably, all of the functional groups A1 to A9 or A1 to A10 are represented in a preferred consortium of the present invention. As discussed above, all bacterial strains are defined by their functions or by their capacity to perform at least one metabolic pathway. Such functions may be accomplished by one or more than one bacterial strain. Accordingly, each functional group comprises one or more, preferably one, bacterial strain. Alternatively, one bacterium can be able to perform a plurality of functions, i.e. can belong to one or more functional group.

In some embodiments, the consortium comprises at least one bacterial strain in each of the A1, A2, A3, A4, A5, A6, A7, A8 and A9 functional groups. Optionally, it further comprises a bacterial strain of functional group A10 and/or a bacterial strain of functional group A11. Optionally, the consortium may comprise a bacterial strain that belongs to more than one functional group of the A1, A2, A3, A4, A5, A6, A7, A8 and A9 functional groups. Then, a particular bacterial strain can belong to 2, 3 or 4 functional groups. In one embodiment, the consortium comprises a bacterial strain that belongs to both of the functional groups A6 and A9, i.e. such bacterial strain being capable of performing metabolic pathways of functional groups A6 and A9, i.e. metabolic pathways 3, 6, 8 and 12. In another embodiment, the consortium comprises a bacterial strain that belongs to both of the functional groups A4 and A7, i.e. such bacterial strain being capable of performing metabolic pathways of functional groups A4 and A7, i.e. metabolic pathways 1, 2 and 4.

Then, if each bacteria strain of the consortium belongs to a different functional group, the consortium can be composed by at least 9 or 10 bacteria.

Alternatively, if a particular bacterial strain belongs to at least two functional groups (e.g. A6 and A9 or A4 and A7), then the consortium may comprise less than 9 or 10 bacterial strains, preferably 8, 7, 6, 5, 4 or 3 bacterial strains.

In addition, the consortium may also comprise more than one bacterial strain for one functional group, the consortium is composed of more than 9 or 10 bacterial strains, preferably 11, 12, 13, 14, 15, 16, 17, 18, 19, 20, 25, 30, 35, 40, 45 or 50 bacteria.

The consortium may further comprise bacterial strains of one or more groups selected from A10, A11, A12, A13, A14 and A15.

Bacterial strains Group (A1) comprises bacteria strains consuming sugars, fibers, and resistant starch, and producing formate and acetate. Such bacteria strains are known and include bacteria of the genera Ruminococcus, Clostridium, Dorea and Eubacterium, such as the species Ruminococcus bromii (ATCC 27255, ATCC 51896), Ruminococcus lactaris (ATCC 29176), Ruminococcus champanellensis (DSM 18848, JCM 17042), Ruminococcus callidus (ATCC 27760), Ruminococcus gnavus (ATCC 29149, ATCC 35913, JCM 6515), Ruminococcus obeum (ATCC 29174, DSM 25238, JCM 31340), Dorea longicatena (DSM 13814, JCM 11232), Dorea formicigenerans (ATCC 27755, DSM 3992, JCM 31256), Clostridium scindens (DSM 5676, ATCC35704) and Eubacterium eligens (ATCC 27750, DSM 3376).

Optionally, Group (A1) comprises bacteria strains consuming sugars, fibers, and resistant starch, producing formate and acetate. Such bacteria strains are known and include bacteria of the genera Ruminococcus, Dorea and Eubacterium such as the species Ruminococcus bromii (ATCC 27255, ATCC 51896), Ruminococcus lactaris (ATCC 29176), Ruminococcus champanellensis (DSM 18848, JCM 17042), Ruminococcus callidus (ATCC 27760), Ruminococcus gnavus (ATCC 29149, ATCC 35913, JCM 6515), Ruminococcus obeum (ATCC 29174, DSM 25238, JCM 31340), Dorea longicatena (DSM 13814, JCM 11232), Dorea formicigenerans (ATCC 27755, DSM 3992, JCM 31256) and Eubacterium eligens (ATCC 27750, DSM 3376).

Group (A2) comprises bacteria strains consuming sugars, starch and acetate, and producing formate and butyrate. Such bacteria strains are known and include bacteria of the genera Faecalibacterium, Roseburia, Eubacterium and Anaerostipes such as the species Faecalibacterium prausnitzii (ATCC 27768, ATCC 27766, DSM 17677, JCM 31915), Anaerostipes hadrus (ATCC 29173, DSM 3319), Roseburia intestinalis (DSM 14610, CIP 107878, JCM 31262), Eubacterium ramulus (ATCC 29099, DSM 15684, JCM 31355) and Eubacterium rectale (DSM 17629).

Optionally, group (A2) comprises bacteria strains consuming sugars, starch and acetate, and producing formate and butyrate. Such bacteria strains are known and include bacteria of the genera Faecalibacterium, Roseburia and Anaerostipes such as the species Faecalibacterium prausnitzii (ATCC 27768, ATCC 27766, DSM 17677, JCM 31915), Anaerostipes hadrus (ATCC 29173, DSM 3319) and Roseburia intestinalis (DSM 14610, CIP 107878, JCM 31262).

Group (A3) comprises bacteria strains consuming sugars and oxygen, producing lactate. Such bacteria strains are known and include bacteria of the genera Lactobacillus, Streptococcus, Escherichia, Lactococcus, Enterococcus such as the species Lactobacillus rhamnosus (ATCC 7469, DSM 20021, JCM 1136), Streptococcus salivarius (ATCC 7073, DSM 20560, JCM 5707), Escherichia coli (ATCC 11775, DSM 30083, JCM 1649), Lactococcus lactis (ATCC 19435, DSM 20481), Enterococcus caccae (ATCC BAA-1240, DSM 19114), and Enterococcus faecalis (ATCC 29212, DSM 2570). Optionally, the bacteria strains are selected from the species Lactobacillus rhamnosus, Streptococcus salivarius, Escherichia coli, Lactococcus lactis and Enterococcus caccae.

Group (A4) comprises bacteria strains consuming sugars, starch, and carbon dioxide, producing lactate, formate and acetate. Such bacteria strains are known and include bacteria of the genus Bifidobacterium and Roseburia, such as the species Bifidobacterium adolescentis (ATCC 15703, DSM 20083, JCM 1251), Bifidobacterium angulatum (ATCC 27535, DSM 20098), Bifidobacterium bifidum (ATCC 29521, DSM 20456, JCM 1255), Bifidobacterium breve (ATCC 1192, DSM 20213), Bifidobacterium catenulatum (ATCC 27539, DSM 16992, JCM 1194), Bifidobacterium dentium (ATCC 27534, DSM 20436, JCM 1195), Bifidobacterium gallicum (ATCC 49850, DSM 20093, JCM 8224), Bifidobacterium longum (ATCC 15707, DSM 20219, JCM 1217), Bifidobacterium pseudocatenulatum (ATCC 27919, DSM 20438, JCM 1200) and Roseburia hominis (DSM 16839).

Optionally, group (A4) comprises bacteria strains consuming sugars, starch, and carbon dioxide, producing lactate, formate and acetate. Such bacteria strains are known and include bacteria of the genus Bifidobacterium, such as the species Bifidobacterium adolescentis (ATCC 15703, DSM 20083, JCM 1251), Bifidobacterium angulatum (ATCC 27535, DSM 20098), Bifidobacterium bifidum (ATCC 29521, DSM 20456, JCM 1255), Bifidobacterium breve (ATCC 1192, DSM 20213), Bifidobacterium catenulatum (ATCC 27539, DSM 16992, JCM 1194), Bifidobacterium dentium (ATCC 27534, DSM 20436, JCM 1195), Bifidobacterium gallicum (ATCC 49850, DSM 20093, JCM 8224), Bifidobacterium longum (ATCC 15707, DSM 20219, JCM 1217), and Bifidobacterium pseudocatenulatum (ATCC 27919, DSM 20438, JCM 1200).

Group (A5) comprises bacteria strains consuming lactate and proteins, producing propionate and acetate. Such bacteria strains are known and include bacteria of the genera Clostridium, Propionibacterium, Veillonella, Megasphaera and Coprococcus such as the species Clostridium aminovalericum (ATCC 13725, DSM 1283, JCM 1421), Clostridium celatum (ATCC 27791, DSM 1785, JCM 1394), Clostridium (Anaerotignum) lactatifermentans (DSM 14214), Clostridium neopropionicum (DSM 3847), Clostridium propionicum (ATCC 25522, DSM 1682, JCM 1430), Megasphaera elsdenii (ATCC 25940, DSM 20460, JCM 1772), Veillonella montpellierensis (DSM 17217), Veillonella ratti (ATCC 17746, DSM 20736, JCM 6512) and Coprococcus catus (ATCC27761).

Optionally, group (A5) comprises bacteria strains consuming lactate and proteins, producing propionate and acetate. Such bacteria strains are known and include bacteria of the genera Clostridium, Propionibacterium, Veillonella, Megasphaera such as the species Clostridium aminovalericum (ATCC 13725, DSM 1283, JCM 1421), Clostridium celatum (ATCC 27791, DSM 1785, JCM 1394), Clostridium (Anaerotignum) lactatifermentans (DSM 14214), Clostridium neopropionicum (DSM 3847), Clostridium propionicum (ATCC 25522, DSM 1682, JCM 1430), Megasphaera elsdenii (ATCC 25940, DSM 20460, JCM 1772), Veillonella montpellierensis (DSM 17217), and Veillonella ratti (ATCC 17746, DSM 20736, JCM 6512).

Group (A6) comprises bacteria strains consuming lactate and starch, producing acetate, butyrate and hydrogen. Such bacteria strains are known and include bacteria of the genera Anaerostipes, Clostridium, and Eubacterium such as the species Anaerostipes caccae (DSM 14662, JCM 13470), Clostridium indolis (ATCC 25771, DSM 755, JCM 1380), Eubacterium hallii (ATCC 27751, DSM 3353, JCM 31263), Eubacterium limosum (ATCC 8486, DSM 20543, JCM 6421), Eubacterium ramulus (ATCC 29099, DSM 15684, JCM 31355).

Group (A7) comprises bacteria strains consuming sugar, starch and formate, producing lactate, formate and acetate. Such bacteria strains are known and include bacteria of the genus Collinsella and Roseburia, such as the species Collinsella aerofaciens (ATCC 25986, DSM 3979, JCM 10188), Collinsella intestinalis (DSM 13280, JCM 10643), Collinsella stercoris (DSM 13279, JCM 10641) and Roseburia hominis (DSM 16839).

Optionally, group (A7) comprises bacteria strains consuming sugar, starch and formate, producing lactate, formate and acetate. Such bacteria strains are known and include bacteria of the genus Collinsella, such as the species Collinsella aerofaciens (ATCC 25986, DSM 3979, JCM 10188), Collinsella intestinalis (DSM 13280, JCM 10643) and Collinsella stercoris (DSM 13279, JCM 10641).

Group (A8) comprises bacteria strains consuming succinate, producing propionate and acetate. Such bacteria strains are known and include bacteria of the genera Phascolarctobacterium, Dialister and Flavonifractor such as the species Phascolarctobacterium faecium (DSM 14760), Dialister succinatiphilus (DSM 21274, JCM 15077), Dialister propionifaciens (JCM 17568) and Flavonifractor plautii (ATCC 29863, DSM 4000).

Optionally, group (A8) comprises bacteria strains consuming succinate, producing propionate and acetate. Such bacteria strains are known and include bacteria of the genera Phascolarctobacterium, Dialister such as the species Phascolarctobacterium faecium (DSM 14760), Dialister succinatiphilus (DSM 21274, JCM 15077) and Dialister propionifaciens (JCM 17568).

Group (A9) comprises bacteria strains consuming sugars, fibers, formate and hydrogen, producing acetate and optionally butyrate. Such bacteria strains are known and include bacteria of the genus Acetobacterium, Blautia, Clostridium, Moorella, Sporomusa and Eubacterium and archaea of the genera Methanobrevibacter, Methanomassiliicoccus such as the species Acetobacterium carbinolicum (ATCC BAA-990, DSM 2925), Acetobacterium malicum (DSM 4132), Acetobacterium wieringae (ATCC 43740, DSM 1911, JCM 2380), Blautia hydrogenotrophica (DSM 10507, JCM 14656), Blautia producta (ATCC 27340, DSM 2950, JCM 1471), Clostridium aceticum (ATCC 35044, DSM 1496, JCM 15732), Clostridium glycolicum (ATCC14880, DSM1288, JCM1401), Clostridium magnum (ATCC 49199, DSM 2767), Clostridium mayombe (ATCC 51428, DSM 2767), Methanobrevibacter smithii (ATCC 35061, DSM 861, JCM 328), Candidatus Methanomassiliicoccus intestinalis, Eubacterium hallii (ATCC 27751, DSM 3353, JCM 31263), Eubacterium limosum (ATCC 8486, DSM 20543, JCM 6421), and Eubacterium ramulus (ATCC 29099, DSM 15684, JCM).

Optionally, group (A9) comprises bacteria strains consuming sugars, fibers, formate and hydrogen, producing acetate and optionally butyrate. Such bacteria strains are known and include bacteria of the genus Blautia and archaea of the genera Methanobrevibacter, Methanomassiliicoccus such as the species Blautia hydrogenotrophica (DSM 10507, JCM 14656), Blautia producta (ATCC 27340, DSM 2950, JCM 1471), Methanobrevibacter smithii (ATCC 35061, DSM 861, JCM 328), Candidatus Methanomassiliicoccus intestinalis. Such bacteria strains further include bacteria of the genera Acetobacterium, Clostridium, Moorella and Sporomusa, such as the species Acetobacterium carbinolicum (ATCC BAA-990, DSM 2925), Acetobacterium malicum (DSM 4132), Acetobacterium wieringae (ATCC 43740, DSM 1911, JCM 2380), Clostridium aceticum (ATCC 35044, DSM 1496, JCM 15732), Clostridium glycolicum (ATCC 14880, DSM 1288, JCM 1401), Clostridium magnum (ATCC 49199, DSM 2767), Clostridium mayombe (ATCC 51428, DSM 2767).

Further Groups It is understood that additional bacteria functional groups (A10) to (A**), in particular (A10), (A11), (A12), (A13), (A14) and/or (A15), may also be present in the compositions described herein. Such groups may further improve the use of the compositions described herein. They may be added to the compositions in the amounts given above.

As an exemplary aspect, group (A10) may be mentioned:

Group (A10) comprises bacteria strains consuming sugars, fibers, and resistant starch, and producing succinate. In one embodiment, group (A10) is selected to cover bacteria producing succinate as a main metabolite. In one further embodiment, group (A10) is selected to cover bacteria producing succinate as a metabolite along with other metabolites, such as acetate and propionate.

Such bacteria strains are known and include bacteria of the genera Alistipes, Bacteroides, Blautia, Barnesiella, Clostridium, Ruminococcus and Prevotella, such as Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii.

Optionally, the bacteria strains are selected from the genera Alistipes, Bacteroides, Blautia, Clostridium, Ruminococcus and Prevotella, such as the species Bacteroides faecis (DSM 24798, JCM 16478), Bacteroides fragilis (ATCC 25285, DSM 2151, JCM 11019), Bacteroides ovatus (ATCC 8483, DSM 1896, JCM 5824), Bacteroides plebeius (DSM 17135, JCM 12973), Bacteroides uniformis (ATCC 8492, DSM 6597, JCM 5828), Bacteroides thetaiotaomicron (ATCC 29148, DSM 2079, JCM 5827), Bacteroides vulgatus (ATCC 8482, DSM 1447, JCM 5826), Bacteroides xylanisolvens (DSM 18836, JCM 15633), Blautia/Clostridium coccoides (ATCC 29236, DSM 935, JCM 1395), Blautia luti (DSM 14534, JCM 17040), Blautia wexlerae (ATCC BAA-1564, DSM 19850, JCM 17041), Clostridium butyricum (ATCC 19398, DSM 10702, JCM 1391), Clostridium bartlettii (ATCC BAA-827, DSM 16795), Ruminococcus callidus (ATCC 27760), Ruminococcus flavefaciens (DSM 25089), Prevotella copri (DSM 18205, JCM 13464), Prevotella stercorea (DSM 18206, JCM 13469), Alistipes finegoldii (DSM 1724, JCM 16770), Alistipes onderdonkii (ATCC BAA-1178, DSM 19147, JCM 16771), and Alistipes shahii (ATCC BAA-1179, DSM 19121, JCM 16773).

In a preferred aspect, group (A10) is selected from bacteria of the genera Alistipes, Bacteroides, Barnesiella, Ruminococcus and Prevotella, such as the species Bacteroides faecis (DSM 24798, JCM 16478), Bacteroidesfragilis (ATCC 25285, DSM 2151, JCM 11019), Bacteroides ovatus (ATCC 8483, DSM 1896, JCM 5824), Bacteroides plebeius (DSM 17135, JCM 12973), Bacteroides uniformis (ATCC 8492, DSM 6597, JCM 5828), Bacteroides thetaiotaomicron (ATCC 29148, DSM 2079, JCM 5827), Bacteroides vulgatus (ATCC 8482, DSM 1447, JCM 5826), Bacteroides xylanisolvens (DSM 18836, JCM 15633), Barnesiella intestinihominis (DSM 21032, JCM 15079), Barnesiella viscericola (DSM 18177, JCM 13660) Ruminococcus callidus (ATCC 27760), Ruminococcus flavefaciens (DSM 25089), Prevotella copri (DSM 18205, JCM 13464), Prevotella stercorea (DSM 18206, JCM 13469), Alistipes finegoldii (DSM 1724, JCM 16770), Alistipes onderdonkii (ATCC BAA-1178, DSM 19147, JCM 16771), and Alistipes shahii (ATCC BAA-1179, DSM 19121, JCM 16773).

Group (A11) comprises bacteria strains consuming proteins and producing acetate or butyrate. Such bacteria strains are known and include bacteria of the genera Clostridium, Coprococcus, Eubacterium, Flavonifractor and Flintibacter, such as the species Clostridium butyricum (ATCC19398, DSM 10702, JCM 1391), Coprococcus eutactus (ATCC 27759), Eubacterium hallii (ATCC 27751, DSM 3353, JCM 31263), Flavonifractor plautii (ATCC 29863, DSM 4000) and Flintibacter butyricum (DSM 27579).

Group (A12) comprises bacteria strains consuming proteins, fibers, starches or sugars and producing biogenic amines such as y-aminobutyric acid (GABA), cadaverine, dopamine, histamine, putrescine, serotonin, spermidine and/or tryptamine. Such bacteria strains are known and include bacteria of the genera Bacteroides, Barnesiella, Bifidobacterium, Clostridium (only tryptamine producers), Enterococcus, Faecalibacterium, Lactobacillus and Ruminococcus (only tryptamine producers), such as the species Bacteroides caccae (DSM 19024, ATCC 43185, JCM 9498), Bacteroides faecis (DSM 24798, JCM 16478), Bacteroides fragilis (DSM 2151, ATCC 25285, JCM 11019), Bacteroides massiliensis (DSM17679), Bacteroides ovatus (DSM 1896, ATCC 8483, JCM 5824), Bacteroides uniformis (DSM 6597, ATCC 8492, JCM 5828), Bacteroides vulgatus (DSM 1447, ATCC 8482), Barnesiella intestinihominis (DSM21032), Bifidobacterium adolescentis (DSM 20083, ATCC 15703) and Lactobacillus plantarum (DSM 2601, ATCC 10241) as GABA producers, Clostridium sporogenes (ATCC 15579), Lactobacillus bulgaricus-52 (NDRI) and Ruminococcus gnavus (ATCC 29149) as tryptamine producers, Acidaminococcus intestini (DSM 21505), Bacteroides massiliensis (DSM 17679), Bacteroides stercoris (ATCC 43183) and Faecalibacterium prausnitzii (DSM 17677) as putrescine producers, and Clostridium bolteae (ATCC BAA-613) as spermidine producers.

Group (A13) comprises bacteria strains consuming primary bile acids and producing secondary metabolites. Such bacteria strains are known and include bacteria of the genera Anaerostipes, Blautia, Clostridium and Faecalibacterium, such as the species Anaerostipes caccae (DSM14662), Blautia hydrogenotrophica (DSM 10507, JCM 14656), Clostridium bolteae (ATCC BAA-613), Clostridium scindens (DSM 5676, ATCC 35704), Clostridium symbiosum (ATCC14940) and Faecalibacterium prausnitzii (DSM 17677)

Group (A14) comprises bacteria strains producing vitamins such as cobalamin (B12), folate (B9) or riboflavin (B2). Such bacteria are known in the art and include bacteria of the genera Bacteroides, Bifidobacterium, Blautia, Clostridium, Faecalibacterium, Lactobacillus, Prevotella and Ruminococcus, such as the species Bacteroides fragilis (DSM 2151, ATCC 25285, JCM 11019), Bifidobacterium adolescentis (DSM 20083, ATCC 15703), Bifidobacterium pseudocatenulatum (ATCC 27919, DSM 20438, JCM 1200), Blautia hydrogenotrophica (DSM 10507, JCM 14656), Clostridium bolteae (ATCC BAA-613), Faecalibacterium prausnitzii (DSM 17677), Lactobacillus plantarum (DSM 2601, ATCC10241), Prevotella copri (DSM 18205, JCM 13464) and Ruminococcus lactaris (ATCC 29176)

Group (A15) comprises bacteria strains consuming mucus. Such bacteria are known in the art and include bacteria of the genera Akkermansia, Bacteroides, Bifidobacterium and Ruminococcus; such as the species Akkermansia muciniphila (ATCC BAA-835), Bacteroides fragilis (DSM 2151, ATCC 25285, JCM 11019), Bacteroides thetaiotaomicron (ATCC 29148, DSM 2079, JCM 5827), Bifidobacterium bifidum (ATCC 29521, DSM 20456, JCM 1255), Ruminococcus gnavus (ATCC 29149, ATCC 35913, JCM 6515) and Ruminococcus torques (ATCC27756).

The bacteria strains as defined herein are in each case identified through classification of the full 16S rRNA gene with assignment for the different taxonomic levels Phylum: 75%, Class: 78.5%, Order: 82%, Family: 86.5%, Genus: 94.5%, Species: 98.65% of sequence similarity, preferably of the whole 16S. Such assignment may be achieved by using SILVA Software (SSURef NR99 128 SILVA) and using the HITdb (Ritari et al., 2015).

Any of the above bacterial strains can be combined together in a consortium as long as all functional group A1 to A9 are represented, optionally with additional groups A10, A11, A12, A13, A14 and/or A15. Such consortium can comprise one or more bacterial strain per functional groups.

Preferably, all of the functional groups A1 to A**, more particularly A1 to A9, optionally with additional groups A10, A11, A12, A13, A14 and/or A15, are represented in a preferred consortium of the present invention. As discussed above, all bacterial strains are defined by their functions. Such functions may be accomplished by one or more than one bacterial strain. Accordingly, each functional group comprises one or more, preferably one, bacterial strains. Alternatively, one bacterium can be able to perform a plurality of functions, i.e. can belong to one or more functional group.

In some embodiments, the consortium comprises at least one bacterial strain in each of the A1, A2, A3, A4, A5, A6, A7, A8 and A9 functional groups. Optionally, it further comprises a bacterial strain of functional group A10 and/or a bacterial strain of functional group A11, A12, A13, A14 and/or A15. Optionally, the consortium may comprise a bacterial strain that belongs to more than one functional group of the A1, A2, A3, A4, A5, A6, A7, A8 and A9 functional groups. Then, a particular bacterial strain can belong to 2, 3 or 4 functional groups. In one embodiment, the consortium comprises a bacterial strain that belongs to both of the functional groups A6 and A9, i.e. such bacterial strain being capable of performing features of functional groups A6 and A9. In another embodiment, the consortium comprises a bacterial strain that belongs to both of the functional groups A4 and A7, i.e. such bacterial strain being capable of performing features of functional groups A4 and A7.

Then, if each bacteria strain of the consortium belongs to a different functional group, the consortium can be composed by at least 9 or 10 bacteria.

Alternatively, if a particular bacterial strain belongs to at least two functional groups (e.g. A6 and A9 or A4 and A7), then the consortium may comprise less than 9 or 10 bacterial strains, preferably 8, 7, 6, 5, 4 or 3 bacterial strains.

In addition, the consortium may also comprise more than one bacterial strain for one functional group, the consortium is composed of more than 9 or 10 bacterial strains, preferably 11, 12, 13, 14, 15, 16, 17, 18, 19, 20, 25, 30, 35, 40, 45 or 50 bacteria.

Then, the composition according to the invention comprises functional groups A1 to A9, optionally optionally in combination with (A10), (A11), (A12), (A13), (A14) and/or (A15) or subsets thereof, wherein functional groups A1 to A15, are:

(A1) Resistant starch degraders,

(A2) Starch degrading-, acetate-consuming and butyrate-producers,

(A3) Oxygen-reducing lactate- and formate-producers,

(A4) Starch-reducing lactate- and formate-producers,

(A5) Protein- and lactate-utilizing and propionate-producers,

(A6) Starch-, protein- and lactate-utilizing and butyrate-producers,

(A7) Starch- and protein-degrading formate- and lactate-producers,

(A8) Protein-, succinate-utilizing, and propionate-producers,

(A9) Hydrogen- and formate-utilizing and acetate-producers,

(A10) is an additional/optional functional group of succinate producers,

(A11) is an additional/optional functional group of protein—utilizer and producers of acetate and butyrate.

(A12) is an additional/optional functional group of proteins, fibers, starches or sugars consumers and biogenic amines producers such as y-aminobutyric acid (GABA), cadaverine, dopamine, histamine, putrescine, serotonin, spermidine and/or tryptamine producers.

(A13) is an additional/optional functional group of primary bile acids consumers and secondary metabolites producers.

(A14) is an additional/optional functional group of vitamins producers such as cobalamin (B12), folate (B9) or riboflavin (B2).

(A15) is an additional/optional functional group of mucus degraders.

Preferably, the composition comprises:
at least one bacterial strain consuming sugars, fibers, and resistant starch, and producing formate and acetate (A1);
at least one bacterial strain consuming sugars, starch and acetate, and producing formate and butyrate (A2);

at least one bacterial strain consuming sugars and oxygen, and producing lactate (A3);
at least one bacterial strain consuming sugars, starch, and carbon dioxide, and producing lactate, formate and acetate (A4),
at least one bacterial strain consuming lactate or proteins, and producing propionate and acetate (A5);
at least one bacterial strain consuming lactate and starch, and producing acetate, butyrate and hydrogen (A6);
at least one bacterial strain consuming sugar, starch, and formate and producing lactate, formate and acetate (A7);
at least one bacterial strain consuming succinate, and producing propionate and acetate (A8); and
at least one bacterial strain consuming sugars, fibers, formate and hydrogen, and producing acetate and optionally butyrate (A9); and
Optionally:
at least one bacterial strain consuming sugars, fibers, and resistant starch, and producing succinate (A10);
at least one bacterial strain consuming proteins and producing acetate and butyrate (A11);
   at least one bacterial strain consuming proteins, fibers, starches or sugars and producing biogenic amines such as y-aminobutyric acid (GABA), cadaverine, dopamine, histamine, putrescine, serotonin, spermidine and/or tryptamine (A12);
   at least one bacterial strain consuming primary bile acids and producing secondary metabolites (A13);
   at least one bacterial strain producing vitamins such as cobalamin (B12), folate (B9) or riboflavin (B2), (A14); and/or
   at least one bacterial strain consuming mucus (A15).

In a first particular aspect, the composition comprises:
at least one bacterial strain consuming sugars, fibers, and resistant starch, and producing formate and acetate (A1);
at least one bacterial strain consuming sugars, starch and acetate, and producing formate and butyrate (A2);
at least one bacterial strain consuming sugars and oxygen, and producing lactate (A3);
at least one bacterial strain consuming sugars, starch, and carbon dioxide, and producing lactate, formate and acetate (A4),
at least one bacterial strain consuming lactate or proteins, and producing propionate and acetate (A5);
at least one bacterial strain consuming lactate, fibers, formate and hydrogen and starch, and producing acetate, butyrate and hydrogen ((A6) and (A9));
at least one bacterial strain consuming sugar, starch, and formate and producing lactate, formate and acetate (A7);
at least one bacterial strain consuming succinate, and producing propionate and acetate (A8); and optionally:
at least one bacterial strain consuming sugars, fibers, and resistant starch, and producing succinate (A10);
at least one bacterial strain consuming proteins and producing acetate and butyrate (A11);
at least one bacterial strain consuming proteins, fibers, starches or sugars and producing biogenic amines such as y-aminobutyric acid (GABA), cadaverine, dopamine, histamine, putrescine, serotonin, spermidine and/or tryptamine (A12);
at least one bacterial strain consuming primary bile acids and producing secondary metabolites (A13);
at least one bacterial strain producing vitamins such as cobalamin (B12), folate (B9) or riboflavin (B2), (A14); and/or
at least one bacterial strain consuming mucus (A15).

In a second particular aspect, the composition comprises:
at least one bacterial strain consuming sugars, fibers, and resistant starch, and producing formate and acetate (A1);
at least one bacterial strain consuming sugars, starch and acetate, and producing formate and butyrate (A2);
at least one bacterial strain consuming sugars and oxygen, and producing lactate (A3);
at least one bacterial strain consuming sugars, starch, formate and carbon dioxide, and producing lactate, formate and acetate ((A4) and (A7)),
at least one bacterial strain consuming lactate or proteins, and producing propionate and acetate (A5);
at least one bacterial strain consuming lactate and starch, and producing acetate, butyrate and hydrogen (A6);
at least one bacterial strain consuming succinate, and producing propionate and acetate (A8); and
at least one bacterial strain consuming sugars, fibers, formate and hydrogen, and producing acetate and optionally butyrate (A9);
optionally:
   at least one bacterial strain consuming sugars, fibers, and resistant starch, and producing succinate (A10);
   at least one bacterial strain consuming proteins and producing acetate and butyrate (A11);
   at least one bacterial strain consuming proteins, fibers, starches or sugars and producing biogenic amines such as y-aminobutyric acid (GABA), cadaverine, dopamine, histamine, putrescine, serotonin, spermidine and/or tryptamine (A12);
   at least one bacterial strain consuming primary bile acids and producing secondary metabolites (A13);
   at least one bacterial strain producing vitamins such as cobalamin (B12), folate (B9) or riboflavin (B2), (A14); and/or
   at least one bacterial strain consuming mucus (A15).

In a third particular aspect, the composition comprises:
at least one bacterial strain consuming sugars, fibers, and resistant starch, and producing formate and acetate (A1);
at least one bacterial strain consuming sugars, starch and acetate, and producing formate and butyrate (A2);
at least one bacterial strain consuming sugars and oxygen, and producing lactate (A3);
at least one bacterial strain consuming sugars, starch, formate and carbon dioxide, and producing lactate, formate and acetate ((A4) and (A7)),
at least one bacterial strain consuming lactate or proteins, and producing propionate and acetate (A5);
at least one bacterial strain consuming lactate, fibers, formate and hydrogen and starch, and producing acetate, butyrate and hydrogen (A6) and (A9);
at least one bacterial strain consuming succinate, and producing propionate and acetate (A8); and optionally:
at least one bacterial strain consuming sugars, fibers, and resistant starch, and producing succinate (A10);
at least one bacterial strain consuming proteins and producing acetate and butyrate (A11);
at least one bacterial strain consuming proteins, fibers, starches or sugars and producing biogenic amines such as y-aminobutyric acid (GABA), cadaverine, dopamine, histamine, putrescine, serotonin, spermidine and/or tryptamine (A12);
at least one bacterial strain consuming primary bile acids and producing secondary metabolites (A13);
at least one bacterial strain producing vitamins such as cobalamin (B12), folate (B9) or riboflavin (B2), (A14); and/or
at least one bacterial strain consuming mucus (A15).

Preferably, such composition comprises:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
at least one bacterial strain of the genus Bifidobacterium or Roseburia (A4), optionally of the genus Bifidobacterium;
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain selected from the genera Anaerostipes, Clostridium and Eubacterium (A6),
at least one bacterial strain of the genus Collinsella or Roseburia (A7), optionally of the genus Collinsella;
at least one bacterial strain selected from the genera Phascolarctobacterium, Flavonifractor and Dialister (A8), optionally selected from the genera Phascolarctobacterium and Dialister; and
at least one bacterial strain selected from the genera Acetobacterium, Blautia, Clostridium, Eubacterium, Moorella, Methanobrevibacter, Methanomassiliicoccus and Sporomusa (A9), optionally selected from the genera Acetobacterium, Blautia, Clostridium, Moorella, Methanobrevibacter, Methanomassiliicoccus and Sporomusa;
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

In a first particular aspect, the composition comprises:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
at least one bacterial strain of the genus Bifidobacterium or Roseburia (A4), optionally of the genus Bifidobacterium;
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain of Eubacterium (A6) and (A9),
at least one bacterial strain of the genus Collinsella or Roseburia (A7), optionally of the genus Collinsella;
at least one bacterial strain selected from the genera Phascolarctobacterium, Flavonifractor and Dialister (A8), optionally selected from the genera Phascolarctobacterium and Dialister; and
optionally at least one bacterial strain selected from the genera Alistipes, Bacteroides, Blautia, Barnesiella, Clostridium, Ruminococcus and Prevotella, optionally Alistipes, Bacteroides, Blautia, Clostridium, Ruminococcus and Prevotella, preferably Alistipes, Bacteroides, Barnesiella, Ruminococcus and Prevotella (A10);
optionally at least one bacterial strain selected from the genera Bacteroides and Brevotella (A11);
optionally at least one bacterial strain selected from the genera Bacteroides, Barnesiella, Bifidobacterium, Clostridium, Enterococcus, Faecalibacterium, Lactobacillus and Ruminococcus (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

In a second particular aspect, the composition comprises:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
at least one bacterial strain of the genus Roseburia (A4) and (A7);
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain selected from the genera Anaerostipes, Clostridium and Eubacterium (A6),
at least one bacterial strain selected from the genera Phascolarctobacterium, Flavonifractor and Dialister (A8), optionally selected from the genera Phascolarctobacterium and Dialister; and
at least one bacterial strain selected from the genera Acetobacterium, Blautia, Clostridium, Eubacterium, Moorella, Methanobrevibacter, Methanomassiliicoccus and Sporomusa (A9), optionally selected from the genera Acetobacterium, Blautia, Clostridium, Moorella, Methanobrevibacter, Methanomassiliicoccus and Sporomusa;
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

In a third particular aspect, the composition comprises:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
at least one bacterial strain of the genus Roseburia (A4) and (A7);
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain of Eubacterium (A6) and (A9),
at least one bacterial strain selected from the genera Phascolarctobacterium, Flavonifractor and Dialister (A8), optionally selected from the genera Phascolarctobacterium and Dialister; and
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

Even more specifically, the composition comprises:
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1), optionally selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);
Faecalibacterium prausnitzii, Anaerostipes hadrus, Roseburia intestinalis Eubacterium ramulus, Eubacterium rectale and any combination thereof (A2), optionally selected from Faecalibacterium prausnitzii, Anaerostipes hadrus, Roseburia intestinalis and any combination thereof (A2);
Lactobacillus rhamnosus, Streptococcus salivarius, Escherichia coli, Lactococcus lactis, Enterococcus faecalis, Enterococcus caccae and any combination thereof (A3), optionally selected from Lactobacillus rhamnosus, Streptococcus salivarius, Escherichia coli, Lactococcus lactis, Enterococcus caccae and any combination thereof (A3);
Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Roseburia hominis and any combination thereof (A4), optionally selected from Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum and any combination thereof (A4);
Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Coprococcus catus, Veillonella ratti and any combination thereof (A5), optionally selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);
Anaerostipes caccae, Clostridium indolis, Eubacterium hallii, Eubacterium limosum, Eubacterium ramulus and any combination thereof (A6);
Collinsella aerofaciens, Collinsella intestinalis, Collinsella stercoris, Roseburia hominis and any combination thereof (A7), optionally selected from Collinsella aerofaciens, Collinsella intestinalis, Collinsella stercoris and any combination thereof (A7);
Phascolarctobacterium faecium, Dialister succinatiphilus, Flavonifractor plautii, Dialister propionifaciens and any combination thereof (A8), optionally selected from Phascolarctobacterium faecium, Dialister succinatiphilus, Dialister propionifaciens and any combination thereof (A8); and
Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis and any combination thereof (A9), optionally selected from Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii, Candidatus Methanomassiliicoccus intestinalis and any combination thereof (A9);
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10), optionally from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii and any combination thereof (A10), preferably from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a particular embodiment, the present invention relates to a composition comprising a consortium as disclosed herein, which comprises Enterococcusfaecalis, belonging to the functional group A3.

In one aspect, the present invention relates to a composition that comprises a consortium comprising Enterococcus faecalis (A3) and
at least one bacterial strain consuming sugars, fibers, and resistant starch, and producing formate and acetate (A1);
at least one bacterial strain consuming sugars, starch and acetate, and producing formate and butyrate (A2);
at least one bacterial strain consuming sugars, starch, and carbon dioxide, and producing lactate, formate and acetate (A4),
at least one bacterial strain consuming lactate or proteins, and producing propionate and acetate (A5);
at least one bacterial strain consuming lactate and starch, and producing acetate, butyrate and hydrogen (A6);
at least one bacterial strain consuming sugar, starch, and formate and producing lactate, formate and acetate (A7);
at least one bacterial strain consuming succinate, and producing propionate and acetate (A8); and
at least one bacterial strain consuming sugars, fibers, formate and hydrogen, and producing acetate and butyrate (A9);
optionally at least one bacterial strain consuming sugars, fibers, and resistant starch, and producing succinate (A10);
optionally at least one bacterial strain consuming proteins and producing acetate and butyrate (A11);
   optionally at least one bacterial strain consuming proteins, fibers, starches or sugars and producing biogenic amines such as y-aminobutyric acid (GABA), cadaverine, dopamine, histamine, putrescine, serotonin, spermidine and/or tryptamine (A12);
   optionally at least one bacterial strain consuming primary bile acids and producing secondary metabolites (A13);
   optionally at least one bacterial strain producing vitamins such as cobalamin (B12), folate (B9) or riboflavin (B2), (A14); and/or
   optionally at least one bacterial strain consuming mucus (A15).

Optionally, the composition may comprise
at least one bacterial strain consuming lactate, fibers, formate and hydrogen and starch, and producing acetate, optionally butyrate and hydrogen (A6) and (A9); and/or
at least one bacterial strain consuming sugars, starch, formate and carbon dioxide, and producing lactate, formate and acetate (A4) and (A7).

More specifically, the composition may comprise:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
Enterococcus faecalis (A3);
at least one bacterial strain of the genus Bifidobacterium or Roseburia (A4), optionally of the genus Bifidobacterium;
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain selected from the genera Anaerostipes, Clostridium and Eubacterium (A6),
at least one bacterial strain of the genus Collinsella or Roseburia (A7), optionally of the genus Collinsella;
at least one bacterial strain selected from the genera Phascolarctobacterium, Flavonifractor and Dialister (A8), optionally selected from the genera Phascolarctobacterium and Dialister; and
at least one bacterial strain selected from the genera Acetobacterium, Blautia, Clostridium, Eubacterium, Moorella, Methanobrevibacter, Methanomassiliicoccus and Sporomusa (A9), optionally selected from the genera Acetobacterium, Blautia, Clostridium, Moorella, Methanobrevibacter, Methanomassiliicoccus and Sporomusa;
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

Optionally, the composition may comprise at least one bacterial strain of Eubacterium (A6) and (A9), and/or at least one bacterial strain of the genus Roseburia (A4) and (A7).

Still more specifically, the present invention relates to a composition comprising a consortium comprising Enterococcus faecalis (A3) and
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1), optionally selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);
Faecalibacterium prausnitzii, Anaerostipes hadrus, Roseburia intestinalis Eubacterium ramulus, Eubacterium rectale and any combination thereof (A2), optionally selected from Faecalibacterium prausnitzii, Anaerostipes hadrus, Roseburia intestinalis and any combination thereof (A2);
Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Roseburia hominis and any combination thereof (A4), optionally selected from Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum and any combination thereof (A4);
Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Coprococcus catus, Veillonella ratti and any combination thereof (A5), optionally selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);
Anaerostipes caccae, Clostridium indolis, Eubacterium hallii, Eubacterium limosum, Eubacterium ramulus and any combination thereof (A6);
Collinsella aerofaciens, Collinsella intestinalis, Collinsella stercoris, Roseburia hominis and any combination thereof (A7), optionally selected from Collinsella aerofaciens, Collinsella intestinalis, Collinsella stercoris and any combination thereof (A7);
Phascolarctobacterium faecium, Dialister succinatiphilus, Flavonifractor plautii, Dialister propionifaciens and any combination thereof (A8), optionally selected from Phascolarctobacterium faecium, Dialister succinatiphilus, Dialister propionifaciens and any combination thereof (A8); and
Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis and any combination thereof (A9), optionally selected from Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii, Candidatus Methanomassiliicoccus intestinalis and any combination thereof (A9);
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10), optionally from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii and any combination thereof (A10), preferably from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a very particular aspect, the composition comprises a consortium comprising Eubacterium limosum (A6) and (A9); and/or Roseburia hominis (A4) and (A7).

In a particular embodiment, the present invention relates to a composition that comprises a consortium which comprises Roseburia hominis, belonging to the functional group A4 and A7.

In one aspect, the present invention relates to a composition comprising a consortium comprising Roseburia hominis (A4) and (A7) and:
at least one bacterial strain consuming sugars, fibers, and resistant starch, and producing formate and acetate (A1);
at least one bacterial strain consuming sugars, starch and acetate, and producing formate and butyrate (A2);
at least one bacterial strain consuming sugars and oxygen, and producing lactate (A3);
at least one bacterial strain consuming lactate or proteins, and producing propionate and acetate (A5);
at least one bacterial strain consuming lactate and starch, and producing acetate, butyrate and hydrogen (A6);
at least one bacterial strain consuming succinate, and producing propionate and acetate (A8); and
at least one bacterial strain consuming sugars, fibers, formate and hydrogen, and producing acetate and optionally butyrate (A9);
optionally at least one bacterial strain consuming sugars, fibers, and resistant starch, and producing succinate (A10); and
optionally at least one bacterial strain consuming proteins and producing acetate and butyrate (A11);
optionally at least one bacterial strain consuming proteins, fibers, starches or sugars and producing biogenic amines such as y-aminobutyric acid (GABA), cadaverine, dopamine, histamine, putrescine, serotonin, spermidine and/or tryptamine (A12);
optionally at least one bacterial strain consuming primary bile acids and producing secondary metabolites (A13);
optionally at least one bacterial strain producing vitamins such as cobalamin (B12), folate (B9) or riboflavin (B2), (A14); and/or
optionally at least one bacterial strain consuming mucus (A15).

Optionally, the consortium may comprise at least one bacterial strain consuming lactate, fibers, formate and hydrogen and starch, and producing acetate, butyrate and hydrogen (A6) and (A9).

More specifically, the composition may comprise a consortium comprising:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
Roseburia hominis (A4) and (A7);
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain selected from the genera Anaerostipes, Clostridium and Eubacterium (A6),
at least one bacterial strain selected from the genera Phascolarctobacterium, Flavonifractor and Dialister (A8), optionally selected from the genera Phascolarctobacterium and Dialister; and
at least one bacterial strain selected from the genera Acetobacterium, Blautia, Clostridium, Eubacterium, Moorella, Methanobrevibacter, Methanomassiliicoccus and Sporomusa (A9), optionally selected from the genera Acetobacterium, Blautia, Clostridium, Moorella, Methanobrevibacter, Methanomassiliicoccus and Sporomusa;
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).
Optionally, the consortium may comprise at least one bacterial strain of Eubacterium (A6) and (A9). Still more specifically, the present invention relates to a consortium comprising Roseburia hominis (A4) and (A7); and
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1), optionally selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);
Faecalibacterium prausnitzii, Anaerostipes hadrus, Roseburia intestinalis Eubacterium ramulus, Eubacterium rectale and any combination thereof (A2), optionally selected from Faecalibacterium prausnitzii, Anaerostipes hadrus, Roseburia intestinalis and any combination thereof (A2);
Lactobacillus rhamnosus, Streptococcus salivarius, Escherichia coli, Lactococcus lactis, Enterococcus faecalis, Enterococcus caccae and any combination thereof (A3), optionally selected from Lactobacillus rhamnosus, Streptococcus salivarius, Escherichia coli, Lactococcus lactis, Enterococcus caccae and any combination thereof (A3);
Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Coprococcus catus, Veillonella ratti and any combination thereof (A5), optionally selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);
Anaerostipes caccae, Clostridium indolis, Eubacterium hallii, Eubacterium limosum, Eubacterium ramulus and any combination thereof (A6);
Phascolarctobacterium faecium, Dialister succinatiphilus, Flavonifractor plautii, Dialister propionifaciens and any combination thereof (A8), optionally selected from Phascolarctobacterium faecium, Dialister succinatiphilus, Dialister propionifaciens and any combination thereof (A8); and
Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis and any combination thereof (A9), optionally selected from Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii, Candidatus Methanomassiliicoccus intestinalis and any combination thereof (A9);
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10), optionally from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii and any combination thereof (A10), preferably from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a very particular aspect, the consortium comprises Eubacterium limosum (A6) and (A9) and/or Enterococcus faecalis (A3).

In a particular embodiment, the present invention relates to a consortium as disclosed herein which comprises Eubacterium limosum, belonging to the functional groups A6 and A9.

In one aspect, the present invention relates to a consortium comprising Eubacterium limosum ((A6) and (A9)) and:
at least one bacterial strain consuming sugars, fibers, and resistant starch, and producing formate and acetate (A1);
at least one bacterial strain consuming sugars, starch and acetate, and producing formate and butyrate (A2);
at least one bacterial strain consuming sugars and oxygen, and producing lactate (A3);
at least one bacterial strain consuming sugars, starch, and carbon dioxide, and producing lactate, formate and acetate (A4),
at least one bacterial strain consuming lactate or proteins, and producing propionate and acetate (A5);
at least one bacterial strain consuming sugar, starch, and formate and producing lactate, formate and acetate (A7);
at least one bacterial strain consuming succinate, and producing propionate and acetate (A8); and
optionally at least one bacterial strain consuming sugars, fibers, and resistant starch, and producing succinate (A10); and
optionally at least one bacterial strain consuming proteins and producing acetate and butyrate (A11);
   optionally at least one bacterial strain consuming proteins, fibers, starches or sugars producing biogenic amines such as y-aminobutyric acid (GABA), cadaverine, dopamine, histamine, putrescine, serotonin, spermidine and/or tryptamine (A12);
   optionally at least one bacterial strain consuming primary bile acids and producing secondary metabolites (A13);
   optionally at least one bacterial strain producing vitamins such as cobalamin (B12), folate (B9) or riboflavin (B2), (A14); and/or
   optionally at least one bacterial strain consuming mucus (A15).

Optionally, the consortium may comprise at least one bacterial strain consuming lactate, fibers, formate and hydrogen and starch, and producing acetate, butyrate and hydrogen (A6) and (A9).

More specifically, the consortium may comprise
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
at least one bacterial strain of the genus Bifidobacterium or Roseburia (A4), optionally of the genus Bifidobacterium;
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
Eubacterium limosum (A6) and (A9);
at least one bacterial strain of the genus Collinsella or Roseburia (A7), optionally of the genus Collinsella;
at least one bacterial strain selected from the genera Phascolarctobacterium, Flavonifractor and Dialister (A8), optionally selected from the genera Phascolarctobacterium and Dialister; and
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

Optionally, the consortium may comprise at least one bacterial strain of Roseburia (A4) and (A7).

Still more specifically, the present invention relates to a consortium comprising Eubacterium limosum (A6) and (A9); and
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1), optionally selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);
Faecalibacterium prausnitzii, Anaerostipes hadrus, Roseburia intestinalis Eubacterium ramulus, Eubacterium rectale and any combination thereof (A2), optionally selected from Faecalibacterium prausnitzii, Anaerostipes hadrus, Roseburia intestinalis and any combination thereof (A2);
Lactobacillus rhamnosus, Streptococcus salivarius, Escherichia coli, Lactococcus lactis, Enterococcus faecalis, Enterococcus caccae and any combination thereof (A3), optionally selected from Lactobacillus rhamnosus, Streptococcus salivarius, Escherichia coli, Lactococcus lactis, Enterococcus caccae and any combination thereof (A3);
Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Roseburia hominis and any combination thereof (A4), optionally selected from Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum and any combination thereof (A4);
Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Coprococcus catus, Veillonella ratti and any combination thereof (A5), optionally selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);
Eubacterium limosum (A6) and (A9);
Collinsella aerofaciens, Collinsella intestinalis, Collinsella stercoris, Roseburia hominis and any combination thereof (A7), optionally selected from Collinsella aerofaciens, Collinsella intestinalis, Collinsella stercoris and any combination thereof (A7);
Phascolarctobacterium faecium, Dialister succinatiphilus, Flavonifractor plautii, Dialister propionifaciens and any combination thereof (A8), optionally selected from Phascolarctobacterium faecium, Dialister succinatiphilus, Dialister propionifaciens and any combination thereof (A8); and
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10), optionally from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii and any combination thereof (A10), preferably from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a very particular aspect, the consortium comprises Roseburia hominis (A4) and (A7) and Enterococcus faecalis (A3) and:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain selected from the genera Anaerostipes, Clostridium and Eubacterium (A6),
at least one bacterial strain selected from the genera Phascolarctobacterium, Flavonifractor and Dialister (A8), optionally selected from the genera Phascolarctobacterium and Dialister; and
at least one bacterial strain selected from the genera Acetobacterium, Blautia, Clostridium, Eubacterium, Moorella, Methanobrevibacter, Methanomassiliicoccus and Sporomusa (A9), optionally selected from the genera Acetobacterium, Blautia, Clostridium, Moorella, Methanobrevibacter, Methanomassiliicoccus and Sporomusa;
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

Still more specifically, the present invention relates to a consortium comprising Roseburia hominis ((A4) and (A7)) and Enterococcus faecalis (A3) and:
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1), optionally selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);
Faecalibacterium prausnitzii, Anaerostipes hadrus, Roseburia intestinalis Eubacterium ramulus, Eubacterium rectale and any combination thereof (A2), optionally selected from Faecalibacterium prausnitzii, Anaerostipes hadrus, Roseburia intestinalis and any combination thereof (A2);
Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Coprococcus catus, Veillonella ratti and any combination thereof (A5), optionally selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);
Anaerostipes caccae, Clostridium indolis, Eubacterium hallii, Eubacterium limosum, Eubacterium ramulus and any combination thereof (A6);
Phascolarctobacterium faecium, Dialister succinatiphilus, Flavonifractor plautii, Dialister propionifaciens and any combination thereof (A8), optionally selected from Phascolarctobacterium faecium, Dialister succinatiphilus, Dialister propionifaciens and any combination thereof (A8); and
Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis and any combination thereof (A9), optionally selected from Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii, Candidatus Methanomassiliicoccus intestinalis and any combination thereof (A9);
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10), optionally from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii and any combination thereof (A10), preferably from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a particular embodiment, the present invention relates to a consortium as disclosed herein which comprises Flavonifractor plautii, belonging to the functional group A8.

In one aspect, the present invention relates to a consortium comprising Flavonifractor plautii (A8) and:
at least one bacterial strain selected from the genera Ruminococcus, Dorea, Clostridium and Eubacterium (A1), optionally selected from the genera Ruminococcus, Dorea and Eubacterium;
at least one bacterial strain selected from the genera Faecalibacterium, Roseburia, Anaerostipes and Eubacterium (A2), optionally selected from the genera Faecalibacterium, Roseburia and Anaerostipes;
at least one bacterial strain selected from the genera Lactobacillus, Streptococcus, Escherichia, Lactococcus and Enterococcus (A3);
at least one bacterial strain of the genus Bifidobacterium or Roseburia (A4), optionally of the genus Bifidobacterium;
at least one bacterial strain selected from the genera Clostridium, Propionibacterium, Veillonella, Coprococcus and Megasphaera (A5), optionally selected from the genera Clostridium, Propionibacterium, Veillonella and Megasphaera;
at least one bacterial strain selected from the genera Anaerostipes, Clostridium and Eubacterium (A6),
at least one bacterial strain of the genus Collinsella or Roseburia (A7), optionally of the genus Collinsella; and
at least one bacterial strain selected from the genera Acetobacterium, Blautia, Clostridium, Eubacterium, Moorella, Methanobrevibacter, Methanomassiliicoccus and Sporomusa (A9), optionally selected from the genera Acetobacterium, Blautia, Clostridium, Moorella, Methanobrevibacter, Methanomassiliicoccus and Sporomusa;
optionally at least one bacterial strain selected from the genera Alistipes, Bacteroides, Blautia, Barnesiella, Clostridium, Ruminococcus and Prevotella (A10), optionally selected from the genera Alistipes, Bacteroides, Blautia, Clostridium, Ruminococcus and Prevotella, preferably Alistipes, Bacteroides, Barnesiella, Ruminococcus and Prevotella;
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Blautia, Clostridium and Faecalibacterium (A13)
optionally at least one bacterial strain selected from the genera Bacteroides, Bifidobacterium, Blautia, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

Still more specifically, the present invention relates to a consortium comprising Flavonifractor plautii (A8) and:
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1), optionally selected from Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1);
Faecalibacterium prausnitzii, Anaerostipes hadrus, Roseburia intestinalis Eubacterium ramulus, Eubacterium rectale and any combination thereof (A2), optionally selected from Faecalibacterium prausnitzii, Anaerostipes hadrus, Roseburia intestinalis and any combination thereof (A2);
Lactobacillus rhamnosus, Streptococcus salivarius, Escherichia coli, Lactococcus lactis, Enterococcus faecalis, Enterococcus caccae and any combination thereof (A3), optionally selected from Lactobacillus rhamnosus, Streptococcus salivarius, Escherichia coli, Lactococcus lactis, Enterococcus caccae and any combination thereof (A3);
Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Roseburia hominis and any combination thereof (A4), optionally selected from Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum and any combination thereof (A4);
Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Coprococcus catus, Veillonella ratti and any combination thereof (A5), optionally selected from Clostridium aminovalericum, Clostridium celatum, Clostridium (Anaerotignum) lactatifermentans, Clostridium neopropionicum, Clostridium propionicum, Megasphaera elsdenii, Veillonella montpellierensis, Veillonella ratti and any combination thereof (A5);
Anaerostipes caccae, Clostridium indolis, Eubacterium hallii, Eubacterium limosum, Eubacterium ramulus and any combination thereof (A6);
Collinsella aerofaciens, Collinsella intestinalis, Collinsella stercoris, Roseburia hominis and any combination thereof (A7), optionally selected from Collinsella aerofaciens, Collinsella intestinalis, Collinsella stercoris and any combination thereof (A7); and
Acetobacterium carbinolicum, Acetobacterium malicum, Acetobacterium wieringae, Blautia hydrogenotrophica, Blautia producta, Eubacterium limosum, Eubacterium hallii, Eubacterium ramulus, Clostridium aceticum, Clostridium glycolicum, Clostridium magnum, Clostridium mayombe, Methanobrevibacter smithii, Candidatus Methanomassiliicoccus intestinalis and any combination thereof (A9), optionally selected from Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii, Candidatus Methanomassiliicoccus intestinalis and any combination thereof (A9);
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10), optionally from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Blautia/Clostridium coccoides, Blautia luti, Blautia wexlerae, Clostridium butyricum, Clostridium bartlettii, Ruminococcus callidus, Ruminococcus flavefaciens, Prevotella copri, Prevotella stercorea, Alistipes finegoldii, Alistipes onderdonkii, and Alistipes shahii and any combination thereof (A10), preferably from Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10);
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris and Faecalibacterium prausnitzii as putrescine producers, and Clostridium bolteae as spermidine producers and any combination thereof (A12)
optionally Anaerostipes caccae, Blautia hydrogenotrophica, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Blautia hydrogenotrophica, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In a particular embodiment, the consortium comprises or essentially consists of:
Ruminococcus bromii (A1), Faecalibacterium prausnitzii (A2), Lactobacillus rhamnosus (A3), Bifidobacterium adolescentis (A4), Anaerotignum lactatifermentans (A5), Eubacterium limosum (A6), Collinsella aerofaciens (A7), Phascolarctobacterium faecium (A8), and Blautia hydrogenotrophica (A9) and optionally Bacteroides xylanisolvens (A10).

Alternatively, the consortium comprises or essentially consists of:
Ruminococcus bromii (A1), Faecalibacterium prausnitzii (A2), Lactobacillus rhamnosus (A3), Bifidobacterium adolescentis (A4), Anaerotignum lactatifermentans (A5), Eubacterium limosum (A6 and A9), Collinsella aerofaciens (A7) and Phascolarctobacterium faecium (A8) and optionally Bacteroides xylanisolvens (A10); Alternatively, the consortium comprises or essentially consists of:
Eubacterium eligens (A1), Roseburia intestinalis (A2), Enterococcus faecalis (A3), Roseburia hominis (A4 and A7), Coprococcus catus (A5), Eubacterium hallii (A6), Flavonifractor plautii (A8), Eubacterium limosum (A9) and optionally Bacteroides xylanisolvens (A10).

Alternatively, the consortium comprises or essentially consists of:
Eubacterium eligens (A1), Roseburia intestinalis (A2), Enterococcus faecalis (A3), Roseburia hominis (A4 and A7), Coprococcus catus (A5), Eubacterium limosum (A6 and A9), and Flavonifractor plautii (A8).

In a particular aspect, the consortium is such that it does not comprise a bacterium from the genus Blautia, nor an archaea of the genus Methanobrevibacter or Methanomassiliicoccus, especially Blautia hydrogenotrophica, Blautia producta, Methanobrevibacter smithii and Candidatus Methanomassiliicoccus intestinalis, particularly when the consortium comprises Eubacterium limosum, particularly when the consortium comprises Eubacterium limosum such as to fulfils the metabolic function of functional group A9, preferably A9 and A6.

In a particular aspect, the consortium is such that it does not comprise a bacterium from the genus Blautia, Acetobacterium, Clostridium, Moorella, and Sporomusa, nor an archaea of the genus Methanobrevibacter or Methanomassiliicoccus, especially Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii and Candidatus Methanomassiliicoccus intestinalis, particularly when the consortium comprises Eubacterium limosum, particularly when the consortium comprises Eubacterium limosum such as to fulfils the metabolic function of functional group A9, preferably A9 and A6.

In a particular aspect, preferably when the consortium comprises an Eubacterium, preferably Eubacterium limosum, the consortium is such that it does not comprise Blautia hydrogenotrophica.

In another particular aspect, the consortium is such that it does not comprise a bacterium from the genus Blautia, especially Blautia hydrogenotrophica and/or Blautia producta, particularly when the consortium comprises an Eubacterium, preferably Eubacterium limosum.

Additionally or alternatively, particularly when the consortium comprises an Eubacterium, preferably Eubacterium limosum, the consortium is such that it does not comprise:
an archaea of the genus Methanobrevibacter or Methanomassiliicoccus, preferably Methanobrevibacter smithii and/or Candidatus Methanomassiliicoccus intestinalis,
a bacterium of the genera Acetobacterium, preferably Acetobacterium carbinolicum, Acetobacterium malicum and/or Acetobacterium wieringae,
a bacterium of the genera Moorella and/or Sporomusa; and/or
   a bacterium selected from Clostridium aceticum, Clostridium glycolicum, Clostridium magnum and/or Clostridium mayombe.

Then, in one embodiment, the consortium comprises or essentially consists in Eubacterium limosum (A6+A9) and:
at least one bacterial strain selected from the genera Ruminococcus, Dorea, Clostridium and Eubacterium (A1);
at least one bacterial strain selected from the genera Faecalibacterium, Roseburia, Anaerostipes and Eubacterium (A2);
at least one bacterial strain selected from the genera Lactobacillus, Streptococcus, Escherichia, Lactococcus and Enterococcus (A3);
at least one bacterial strain of the genus Bifidobacterium or Roseburia (A4);
at least one bacterial strain selected from the genera Clostridium, Propionibacterium, Veillonella, Coprococcus and Megasphaera (A5);
at least one bacterial strain of the genus Collinsella or Roseburia (A7);
at least one bacterial strain selected from the genera Phascolarctobacterium, Flavonifractor and Dialister (A8); and
optionally at least one bacterial strain selected from the genera Alistipes, Bacteroides, Barnesiella, Clostridium, Ruminococcus and Prevotella (A10);
optionally at least one bacterial strain selected from the genera Clostridium, Coprococcus, Eubacterium, Flavonifractor and Flintibacter (A11);
optionally at least one bacterial strain selected from the genera Bacteroides, Barnesiella, Bifidobacterium, Clostridium (only tryptamine producers), Enterococcus, Faecalibacterium, Lactobacillus and Ruminococcus (only tryptamine producers) (A12);
optionally at least one bacterial strain selected from the genera Anaerostipes, Clostridium and Faecalibacterium (A13)
optionally at least one bacterial strain selected from the genera Bacteroides, Bifidobacterium, Clostridium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

Particularly, the consortium comprises or essentially consists in Eubacterium limosum (A6+A9) and:
at least one bacterial strain selected from the genera Ruminococcus, Dorea, and Eubacterium (A1);
at least one bacterial strain selected from the genera Faecalibacterium, Roseburia, Anaerostipes and Eubacterium (A2);
at least one bacterial strain selected from the genera Lactobacillus, Streptococcus, Escherichia, Lactococcus and Enterococcus (A3);
at least one bacterial strain of the genus Bifidobacterium or Roseburia (A4);
at least one bacterial strain selected from the genera Propionibacterium, Veillonella, Coprococcus and Megasphaera (A5);
at least one bacterial strain of the genus Collinsella or Roseburia (A7);
at least one bacterial strain selected from the genera Phascolarctobacterium, Flavonifractor and Dialister (A8); and
optionally at least one bacterial strain selected from the genera Alistipes, Bacteroides, Barnesiella, Ruminococcus and Prevotella (A10);
optionally at least one bacterial strain selected from the genera Coprococcus, Eubacterium, Flavonifractor and Flintibacter (A11);
optionally at least one bacterial strain selected from the genera Bacteroides, Barnesiella, Bifidobacterium, (only tryptamine producers), Enterococcus, Faecalibacterium, Lactobacillus and Ruminococcus (only tryptamine producers) (A12);
optionally at least one bacterial strain selected from the genera Anaerostipes, and Faecalibacterium (A13)
optionally at least one bacterial strain selected from the genera Bacteroides, Bifidobacterium, Faecalibacterium, Lactobacillus, Prevotella and Ruminococcus (A14); and
optionally at least one bacterial strain selected from the genera Akkermansia, Bacteroides, Bifidobacterium and Ruminococcus (A15).

More specifically, the consortium comprises or essentially consists in Eubacterium limosum (A6+A9) and:
Ruminococcus bromii, Ruminococcus lactaris, Ruminococcus champanellensis, Ruminococcus callidus, Ruminococcus gnavus, Ruminococcus obeum, Clostridium scindens, Dorea longicatena, Dorea formicigenerans, Eubacterium eligens and any combination thereof (A1),
Faecalibacterium prausnitzii, Anaerostipes hadrus, Roseburia intestinalis Eubacterium ramulus, Eubacterium rectale and any combination thereof (A2),
Lactobacillus rhamnosus, Streptococcus salivarius, Escherichia coli, Lactococcus lactis, Enterococcus faecalis, Enterococcus caccae and any combination thereof (A3),
Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Roseburia hominis and any combination thereof (A4),
Clostridium aminovalericum, Clostridium celatum, Clostridium lactatifermentans, Clostridium neopropionicum, Clostridium propionicum, Megasphaera elsdenii, Veillonella montpellierensis, Coprococcus catus, Veillonella ratti and any combination thereof (A5);
Collinsella aerofaciens, Collinsella intestinalis, Collinsella stercoris, Roseburia hominis and any combination thereof (A7),
Phascolarctobacterium faecium, Dialister succinatiphilus, Flavonifractor plautii, Dialister propionifaciens and any combination thereof (A8); and
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Clostridium butyricum, Clostridium bartlettii, Ruminococcus callidus, Ruminococcus flavefaciens, Prevotella copri, Prevotella stercorea, Alistipes finegoldii, Alistipes onderdonkii, Alistipes shahii and any combination thereof (A10)
optionally Clostridium butyricum, Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Clostridium sporogenes, Lactobacillus bulgaricus-52 and Ruminococcus gnavus as tryptamine producers, Acidaminococcus intestini, Bacteroides massiliensis, Bacteroides stercoris, Enterococcus faecalis, Enterococcus faecium and Faecalibacterium prausnitzii as putrescine producers, and Clostridium bolteae as spermidine producers and any combination thereof (A12)
optionally Anaerostipes caccae, Clostridium bolteae, Clostridium scindens, Clostridium symbiosum and Faecalibacterium prausnitzii and any combination thereof (A13)
optionally Bacteroides fragilis, Bifidobacterium adolescentis, Bifidobacterium pseudocatenulatum, Clostridium bolteae, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

Particularly, the consortium comprises or essentially consists in Eubacterium limosum (A6+A9) and:
Ruminococcus bromii, Ruminococcus lactaris, Ruminococcus champanellensis, Ruminococcus callidus, Ruminococcus gnavus, Ruminococcus obeum, Dorea longicatena, Dorea formicigenerans, Eubacterium eligens and any combination thereof (A1),
Faecalibacterium prausnitzii, Anaerostipes hadrus, Roseburia intestinalis Eubacterium ramulus, Eubacterium rectale and any combination thereof (A2),
Lactobacillus rhamnosus, Streptococcus salivarius, Escherichia coli, Lactococcus lactis, Enterococcus faecalis, Enterococcus caccae and any combination thereof (A3),
Bifidobacterium adolescentis, Bifidobacterium angulatum, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Roseburia hominis and any combination thereof (A4),
Megasphaera elsdenii, Veillonella montpellierensis, Coprococcus catus, Veillonella ratti and any combination thereof (A5);
Collinsella aerofaciens, Collinsella intestinalis, Collinsella stercoris, Roseburia hominis and any combination thereof (A7),
Phascolarctobacterium faecium, Dialister succinatiphilus, Flavonifractor plautii, Dialister propionifaciens and any combination thereof (A8); and
optionally Bacteroides faecis, Bacteroides fragilis, Bacteroides ovatus, Bacteroides plebeius, Bacteroides uniformis, Bacteroides thetaiotaomicron, Bacteroides vulgatus, Bacteroides xylanisolvens, Barnesiella intestinihominis, Barnesiella viscericola, Ruminococcus callidus, Ruminococcus flavefaciens, Prevotella copri, Prevotella stercorea, Alistipes finegoldii, Alistipes onderdonkii, Alistipes shahii and any combination thereof (A10)
optionally Coprococcus eutactus, Eubacterium hallii, Flavonifractor plautii and Flintibacter butyricum and any combination thereof (A11);
optionally Bacteroides caccae, Bacteroides faecis, Bacteroides fragilis, Bacteroides massiliensis, Bacteroides ovatus, Bacteroides uniformis, Bacteroides vulgatus, Barnesiella intestinihominis, Bifidobacterium adolescentis and Lactobacillus plantarum as GABA producers, Lactobacillus bulgaricus-52 and Ruminococcus gnavus as tryptamine producers, Acidaminococcus intestini, Bacteroides massiliensis, Bacteroides stercoris, Enterococcus faecalis, Enterococcus faecium and Faecalibacterium prausnitzii as putrescine producers and any combination thereof (A12)
optionally Anaerostipes caccae, and Faecalibacterium prausnitzii and any combination thereof (A13)
optionally Bacteroides fragilis, Bifidobacterium adolescentis, Bifidobacterium pseudocatenulatum, Faecalibacterium prausnitzii, Lactobacillus plantarum, Prevotella copri and Ruminococcus lactaris and any combination thereof (A14); and
optionally Akkermansia muciniphila, Bacteroides fragilis, Bacteroides thetaiotaomicron, Bifidobacterium bifidum, Ruminococcus gnavus and Ruminococcus torques and any combination thereof (A15).

In pure culture, the functions of single bacteria strains (A1) to (A15) may be bidirectional. For example, (A7) may either produce or consume formate. However, when combined in the inventive compositions, the bacteria strains show the properties discussed herein, consuming intermediate metabolites (succinate, lactate, formate) and producing end metabolites (acetate, propionate, butyrate).

Any bacterial strains described herein may be assemble as a synthetic and symbiotic consortium which is characterized by a combination of microbial activities forming a trophic chain from complex fiber metabolism to the canonical final SCFAs (Short chain fatty acids) found in the healthy intestine: acetate, propionate and butyrate. The trophic completeness of the consortium prevents the accumulation of potentially toxic or pain inducing products such as H₂, lactate, formate and succinate. Activities are screened by functional characterization on different substrates of the human gut microbiota. However, type and origin of strains can be selected according to the targeted level of complexity of the synthetic and symbiotic consortia in order to recompose a complex microbiota replacing FMT. The different bacteria strains (A1) to (A15), particularly (A1) to (A9), grow as a consortium, ensuring degradation of complex polysaccharides usually found in the gut (resistant starch, xylan, arabinoxylan and pectin), reutilization of sugars released, removal of O₂ traces for maintenance of anaerobiosis essential for growth, production of key intermediate metabolites (acetate, lactate, formate, CO2 and H2), reutilization of all intermediate metabolites and production of end metabolites found in a healthy gut (acetate, propionate and butyrate). The microbial symbiotic consortia exclusively produce end-fermentation products that are beneficial and used by the host for different functions such as acetate (energy source for heart and brain cells), propionate (metabolized by the liver) and butyrate (the main source of energy for intestinal epithelial cells).

Method of Manufacturing

Manufacturing methods of the designed consortia of a plurality of selected bacterial strains have been previously described in WO2018189284; the content thereof being incorporated by reference. The manufacturing methods as described in WO2018189284 are typically performed on a laboratory scale up to a volume of 200 ml of culture suspension in a bioreactor.

As already mentioned, the present invention relates to a method suitable for a production at an industrial scale.

The invention concerns an in vitro method for manufacturing a consortium of at least three different bacterial strains as disclosed above, wherein the method of manufacturing comprises the steps of:

I. providing an inoculum consortium comprising said at least three bacterial strains,

wherein the inoculum is obtained from a prior continuous anaerobic co-cultivation process of said at least three bacterial strains, at least until a stable microbial profile and a stable metabolic profile are obtained, and

wherein the inoculum is provided as a preserved inoculum, preferably a lyophilized or cryopreserved inoculum;

II. adding the inoculum to a culture medium;

III. multiplying said at least three bacterial strains by co-cultivation in the culture medium at least until a stable microbial profile and a stable metabolic profile are obtained, wherein this step is performed in an anaerobic batch or fed-batch fermentation process;

IV. harvesting the consortium of bacterial strains; and

V. optionally, subjecting the harvested consortium to one or more further processing steps.

Step I

The in vitro assembled consortia used as inoculum are obtainable and in particular established from single strain cultures by including a step of continuous co-cultivation as described below.

Continuous co-cultivation ensures as described herein a balanced amount of each of the bacterial strains of the consortium or of each of the selected functional groups as a plurality of selected strains and the establishment of metabolic interactions, thereby providing metabolic interactions, such as cross-feeding, resulting in a higher amount of the plurality of bacterial strains and stabilization of the relative abundance of the functional groups or bacterial strains present in the consortium. Furthermore, an increased resistance to stress, such as stabilization through cryopreservation or lyophilisation of the single strains and the mixes thereof, has been observed. Continuous co-cultivation in combination with the stabilization through cryopreservation or lyophilisation lead to a consortium inoculum suitable for preparing a final product (the consortium) in a reproducible way at an industrial scale and with high yield and stability of the obtained product. The inventors believe that this step of continuous co-cultivation is mandatory for the establishment of interaction and collaboration between bacteria, in particular to establish cross-feeding processes and comparable growth rates.

Accordingly, in some embodiments of step I of the method, the sample of the consortium or the consortium inoculum is obtained from a prior continuous anaerobic co-cultivation process of the selected bacterial strains at least until a stable microbial profile and a stable metabolic profile characteristic of the in vitro assembled consortium inoculum had been established.

In one embodiment, in step I, the continuous anaerobic co-cultivation process of the selected bacterial strains is preceded by a batch fermentation process. Preferably, such batch fermentation process is a co-cultivation batch fermentation process. Alternatively, the batch fermentation process comprises individual batch fermentation of single strains.

Some embodiments of the method of manufacturing the in vitro assembled consortia of the present invention, the method comprises a preparatory stage for manufacturing the inoculum provided in step I of the method disclosed herein. In a particular embodiment, the method according to the invention comprises a preparatory stage that comprises the steps of:

(a) providing single strain samples of the selected viable, live bacterial strains,

(b) inoculating the selected strains into the dispersing medium in a bioreactor thereby forming a culture suspension and co-cultivating the culture suspension in an anaerobic continuous co-cultivation,

(c) harvesting the consortium of the bacterial strains from the bioreactor after the culture-suspension has established a stable microbial profile and a stable metabolic profile,

(d) optionally subjecting the harvested consortium of the bacterial strains to post-treatment steps. Preferably, the continuous anaerobic co-cultivation in step b) is preceded by a batch fermentation step. Such batch fermentation process is a co-cultivation batch fermentation process.

Accordingly, the process may comprise:

(a) providing single strain samples of the selected viable, live bacterial strains,

(b) inoculating the selected strains into the dispersing medium in a bioreactor thereby forming a culture suspension and co-cultivating the culture suspension in an anaerobic batch co-cultivation followed by an anaerobic continuous co-cultivation,

(c) harvesting the consortium inoculum of the bacterial strains from the bioreactor after the culture-suspension has established a stable microbial profile and a stable metabolic profile,

(d) optionally subjecting the harvested consortium inoculum of the bacterial strains to further processing steps.

Optionally, the step (a) of the preparatory stage comprises:

(a1) providing and separately cultivating said single strain samples in the presence of a substrate specific for each of said strains thereby obtaining single-strain cultures, and

(a2) combining said single-strain cultures of (a1) into a culture-suspension and co-cultivating them under anaerobic conditions in the presence of a dispersing medium. Preferably, step (a2) is terminated once intermediate metabolites, for example such as succinate, formate and lactate, are each below 15 mM.

In step (a1), the cultivation can be a batch fermentation process or a fed-batch fermentation process. In step (a2), the co-cultivation comprises an anaerobic continuous co-cultivation. Preferably, the continuous anaerobic co-cultivation is preceded by a batch fermentation step. Such batch fermentation process is a co-cultivation batch fermentation process.

The composition of the dispersing or culture medium can be designed by the skilled person in the art, taking into account the requirements of bacterial strains of the consortium.

In particular, the dispersing or culture medium comprises substrates or nutrients selected from simple sugars carbon (glucose, galactose, maltose, lactose, sucrose, fructose, cellobiose), “fibers” (preferably pectin, arabinogalactan, beta-glucan, soluble starch, resistant starch, fructo-oligosacharides, galacto-oligosacharides, xylan, arabinoxylans, cellulose), proteins (preferably yeast extract, casein, skimmed milk, peptone), co-factors (short chain fatty acids, hemin, FeSO4), vitamins (preferably biotin or D-(+)-Biotin (Vit. H), Cobalamin (Vit. B12), 4-aminobenzoic acid or p-aminobenzoic acid (PABA), folic acid (Vit. B11/B9), pyridoxamine hydrochloride (Vit. B6)), minerals (preferably sodium bicarbonate, potassium phosphate dibasic, potassium phosphate monobasic, sodium chloride, ammonium sulfate, magnesium sulfate, calcium chloride) and reducing agents (preferably cysteine, titanium(III)-citrate, yeast extract, sodium thioglycolate, dithiothreitol, sodium sulphide, hydrogen sulphite, ascorbate), guar gum, glycerol, potato starch, rice starch, pea starch, corn starch, wheat starch, inulin, succinate, formate, lactate, iron sulfate, tryptone, fucose, acetate, mucus, trehalose, mannitol, polysorbate and any combination thereof.

Preferably, a pH value is adjusted within a range of pH 5-8, preferably pH 5-7, more particularly a range of pH 5.5-7, even more preferably of pH 5.5-6.5.

Preferably, after a duration of 1 or 2 days of co-cultivation, half of the volume of the culture—suspension is replaced by the same volume of fresh dispersing medium or the same volume of medium is added (i.e. double the fermentation volume).

The invention also concerns an in vitro method for manufacturing an inoculum of at least three bacterial strains as disclosed above, wherein the method of manufacturing comprises the steps of:

(a) providing single bacterial strain samples,

(b) inoculating the single bacterial strains into a single culture medium and co-cultivating the bacterial strains in the culture medium by an anaerobic continuous co-cultivation process at least until a stable microbial profile and a stable metabolic profile is reached,

(c) harvesting the consortium inoculum comprising the bacterial strains, and

(d) subjecting the harvested consortium inoculum of the bacterial strains to a preservation treatment, preferably cryopreservation or lyophilisation.

Preferably, in step b):
the bacterial strains enable to maintain concentrations in the culture medium of intermediate metabolites of the trophic network, preferably selected from formate, lactate and succinate and mixtures thereof, below a concentration inhibiting proliferation of at least one bacterial strain of the consortium inoculum;
the bacterial strains enable to maintain concentrations in the culture medium of inhibitory by-products of the trophic network, preferably selected from H₂, and 02 and mixtures thereof, below a concentration inhibiting proliferation of at least one bacterial strain of the consortium inoculum.

Preferably, in step b), the anaerobic continuous co-cultivation is preceded by a step of batch fermentation co-cultivation.

Preferably, in step d), the consortium inoculum is harvested during the late exponential phase of growth or at the beginning of the stationary phase of growth of the bacterial cells.

Then, the invention also concerns an inoculum obtainable or obtained by any method disclosed here above, especially by the in vitro method for manufacturing an inoculum as disclosed herein. The invention also relates to the use of such an inoculum for preparing a consortium of viable bacterial strains, in particular using the method according to the invention.

Preferably, the inoculum of step I is a stabilized inoculum, i.e. having a stable microbial and/or a stable metabolic.

Optionally, the harvested consortium inoculum comprising the selected bacterial strains may be subjected to a preservation-treatment, preferably handled and stored under protection from oxygen, such preservation-treatment being selected from cryopreservation and lyophilization.

Preferably, in step d) the consortium inoculum is submitted to a post-treatment or to one or more further processing step.

In a particular embodiment, the consortium inoculum of step I is cryopreserved in glycerol.

In some of these and of other embodiments of step I, the consortium inoculum is obtained as a preserved inoculum, preferably selected from a cryopreserved inoculum or a lyophilised inoculum.

In one embodiment, the inoculum is submitted to a post-treatment of cryopreservation that comprises the steps of:
mixing the harvested culture-suspension with a cryoprotective solution in particular obtaining a 1:1(v/v) mixture of culture-suspension and glycerol or
centrifuging the harvested culture-suspension and resuspending an obtained pellet in a mixture of the cryoprotective solution and the dispersing medium, in particular in a 1:1 (v/v) mixture of glycerol and the dispersing medium
shock freezing with liquid N2 or gradually freeze to a storage temperature of at least −20° C.

In one embodiment, the inoculum is submitted to a post-treatment of lyophilisation that comprises the steps of:
centrifuging the harvested culture-suspension and wash an obtained pellet with a buffer solution
resuspending the pellet in a lyophilisation solution and lyophilize
subsequent storage at a temperature of 4° C. or lower, or at room temperature.

Step II

In one embodiment, in step II of any method disclosed herein, a cryopreserved consortium inoculum is thawed, preferably at room temperature or at any temperature suitable for bacterial strain recovery, before the inoculation of the bioreactor.

Alternatively, a lyophilized inoculum is re-suspended in the dispersing medium, before the inoculation of the bioreactor.

Preferably, the consortium inoculum is inoculated into the bioreactor in an inoculation ratio of 0.1-25% (v/v), in particular with a 0.5-2% (v/v).

Step III

Preferably, in step III of any method disclosed herein:
the bacterial strains enable to maintain concentrations of intermediate metabolites in the culture medium, preferably selected from formate, lactate and succinate and mixtures thereof, below a concentration inhibiting proliferation of at least one bacterial strain of the consortium;
the bacterial strains enable to maintain concentrations in the culture medium of inhibitory by-products of the trophic network, preferably selected from H₂, and 02 and mixtures thereof, below a concentration inhibiting proliferation of at least one bacterial strain of the consortium.

In some embodiments, step III is performed as a fed-batch fermentation process comprising two or more sub-steps of batch cultivation, in particular for a duration of 12 up to 24 or up to 48 hours.

Preferably, between each of the sub-steps, a further portion of a dispersing medium providing one or more of the complex compounds, nutrients or substrates, preferably selected from sugars, starches, fibers and proteins, is added to the bioreactor.

In one aspect, the co-cultivation is performed using a carrier material biofilm formation and/or physical entrapment of the said bacteria. Materials for such carrier are preferably alginate, k-Carrageenan, chitosan, gelatin gel, xanthan/gellan. In particular, step III is performed as a two-step fed-batch fermentation process comprising the steps of:

III-1 batch fermentation for the duration of one day, in particular for 24 hours, with a dilution of inoculum into the dispersing medium ranging from 1% to 20% of inoculum to dispersing medium (v/v);

III-2 addition a volume of dispersing medium equal to the volume of the culture-suspension in the bioreactor; and

III-3 continuation of the fermentation for another day, in particular for a further 24 hours.

Preferably, the medium of step I and II, has the same or similar composition to the medium of step Ill. In one embodiment, such medium comprises glycerol, preferably so as to enhance butyrate production. The enhancement of butyrate production in the presence of glycerol can be monitored by any method known in the art.

In some embodiments, step I and/or step III is performed at least until a stable microbial and/or a stable metabolic is reached. This means that step II or IV can be performed right after the establishment or monitoring of a stable microbial and/or stable metabolic profile, or following a certain period of time after the establishment or monitoring of the stable microbial and/or stable metabolic profile, for example such as 1, 2, 3 or 4 days after the monitoring and the establishment of the stable microbial and/or stable metabolic profile. In a particular embodiment, step II or IV can be performed at the time of at least 7 full medium renewals in the continuously operated bioreactor.

In some embodiments, in step III or prior to step IV, one or more parameter regarding the microbial profile and/or regarding the metabolic profile of the culture suspension is measured. Optionally, the measured value of the one or more parameter is compared to a standard value of said one or more parameter. Preferably the standard value of said one or more parameter corresponds to the mean value as measured in a culture-suspension comprising the dispersing medium and the selected bacterial strains grown in an anaerobic continuous co-cultivation until said measured value has stabilized over a period of at least 2, 3, 4, 5, 6, 7, 8, 9 or 10 days, preferably 3 days. In a particular embodiment, step II or IV can be performed at the time of at least 7 full medium renewals in the continuously operated bioreactor.

Particularly, the standard value of the one or more parameter corresponds to a standard value selected from the group consisting of:
a concentration of succinate below 15 mM, 10 mM, 5 mM, 1 mM or 0.1 mM
a concentration of formate below 15 mM, 10 mM, 5 mM, 1 mM or 0.1 mM
a concentration of lactate below 15 mM, 10 mM, 5 mM, 1 mM or 0.1 mM
a concentration of acetate above 10 mM, 20 mM or 40 mM
a concentration of propionate above 5 mM, 10 mM or 15 mM
a concentration of butyrate above 5 mM, 10 mM or 15 mM

Preferably, the standard value of the one or more parameter corresponds to a standard value selected from the group consisting of:
a concentration of succinate below 15 mM, 10 mM, 5 mM, 1 mM or 0.1 mM
a concentration of formate below 15 mM, 10 mM, 5 mM, 1 mM or 0.1 mM
a concentration of lactate below 15 mM, 10 mM, 5 mM, 1 mM or 0.1 mM
a concentration of acetate above 10 mM, 20 mM or 40 mM
a concentration of propionate above 5 mM, 10 mM or 15 mM
a concentration of butyrate above 5 mM, 10 mM or 15 mM
a redox value below −300 mV, −350 mV or −400 mV,
an optical density above 1.5, 2 or 3
a viability of over 50%, 60% or 70%
an abundance of bacterial strains of 10⁵-10¹⁴ 16S rRNA gene copies per ml, and
a concentration of oxygen below 150 ppm and/or hydrogen below 10′000 ppm.

In one embodiment, a stable metabolic profile fulfils one or more of the following criteria:
a concentration of one or more of the intermediate metabolites formate, lactate, succinate in the dispersing medium are each below 15 mM, in particular below 10 mM, 5 mM, 1 mM or more particular below 0.1 mM;
a concentration of one or more of propionate and butyrate are above 5 mM, in particular above 10 mM, more particular above 15 mM and wherein the concentration of acetate is above 10 mM, in particular above 20 mM, more particular above 40 mM.

Preferably, a stable metabolic profile fulfils one or more of the following criteria:
a concentration of one or more of the intermediate metabolites, preferably selected from formate, lactate, succinate and mixtures thereof, in the medium are each below 15 mM, in particular below 10 mM, 5 mM, 1 mM or more particular below 0.1 mM.
a concentration of one or more of the end metabolites, preferably selected from propionate, butyrate, acetate and any mixtures thereof, are above 5 mM, in particular above 10 mM, more particular above 15 mM, 20 mM or 40 mM.
In one embodiment, a stable microbial profile exhibits an abundance of each of the bacterial strains in the consortium of 10⁵-10¹⁴ 16S rRNA gene copies per ml of the culture suspension or medium.
The concentration of bacteria strains in the inoculum or in the final product (the consortium) may vary over a broad range. Typically, in the inoculum of an in vitro assembled consortium or in the consortium, the bacterial strains of the consortium, especially of each functional group, are present in a concentration below 10¹⁴ 16S rRNA gene copies per ml of co-cultivated culture-suspension at the time of harvest in the inoculum provided in step I. Typically, each group or bacterium of the inoculum or consortium is present in a concentration above 10⁵ 16S rRNA gene copies per ml of composition, preferably above 10⁶ 16S rRNA gene copies per ml of composition, particularly preferably above 10⁸ 16S rRNA gene copies per ml of composition. Preferably, the concentration of bacteria strains is quantified by qPCR.

Step IV

In some embodiments, in step IV, the bacterial strains of the consortium are harvested during the late exponential phase of growth or at the beginning of the stationary phase of growth of the bacterial cells. Preferably, the harvesting step is performed before at least one of the nutrients or substrates has been completely degraded by a bacterial strain of the consortium.

In a particular embodiment, a sample of the harvested consortium in step IV is used directly or is preserved. Then, the sample may be used to prepare a pharmaceutical composition, in particular a composition used as a drug for the treatment of a disease or a disorder.

Optionally, the preserved sample could subsequently be used as the inoculum of step I in another round of performing the method according to the invention.

Step V

Optionally, the harvested consortium of step IV comprising the selected bacterial strains may be subjected to a preservation-treatment, preferably handled and stored under protection from oxygen, such preservation-treatment being selected from cryopreservation and lyophilization.

In one embodiment, the method comprises a step V, which comprises subjecting the harvested consortium to one or more post-treatment steps or to one of more further processing steps.

In some of these and of other embodiments of step V, the consortium is preserved by cryopreservation or a lyophilisation.

In one embodiment, the post-treatment or further processing step of cryopreservation comprises the steps of:
mixing the harvested culture-suspension with a cryoprotective solution in particular obtaining a 1:1(v/v) mixture of culture-suspension and cryopreservant, preferably such as glycerol, or
centrifuging the harvested culture-suspension and resuspending an obtained pellet in a mixture of the cryoprotective solution and the dispersing medium, in particular in a 1:1 (v/v) mixture of cryopreservant, preferably such as glycerol and the dispersing medium
shock freezing with liquid N2 or gradually freeze to a storage temperature of at least −20° C., in particular at 20° C. to −80° C.,

In one embodiment, the post-treatment or further processing step of lyophilisation comprises the steps of:
centrifuging the harvested culture-suspension and wash an obtained pellet with a buffer solution
resuspending the pellet in a lyophilisation solution and lyophilize
subsequent storage at a temperature of 4° C. or lower, or at room temperature.

In one embodiment, the post-treatment or further processing step of cryopreservation comprises the steps of:
inoculating the consortium on a gel or polymer containing or suspended in nutritive medium
growing the consortium as a biofilm on the carrier gel
subsequent storage at a temperature of 4° C. or lower.

It was observed that exemplary in vitro assembled consortia of bacterial strains such as described in WO2018189284 stabilize towards the same microbial and same metabolic profiles as the originally inoculated in vitro assembled consortium provided that the consortia during continuous co-cultivation fulfil the criteria of
(a) converting the selected nutrients to end metabolites directly or—indirectly via intermediate metabolites—and
(b) avoiding an accumulation of intermediate metabolites to an inhibitory concentration.

Surprisingly, it was found that the in vitro assembled consortium comprising at least three bacterial strains defining a consortium as detailed above or a plurality of functional groups designed for fulfilling criteria (a) and (b) reproducibly stabilize not only during anaerobic continuous co-cultivation for preparing the inoculum but also during anaerobic batch co-cultivation for preparing/producing the consortium, with respect to its microbial composition, thereby forming a characteristic microbial and metabolic profile of the given consortium. Accordingly, during anaerobic batch co-cultivation, the concentrations of intermediate metabolites (if any) and end metabolites stabilize such as to re-establish a characteristic metabolic profile of the given consortium, too. This unexpected effect can be reached by the specific step of production combining a first step of continuous co-cultivation, followed by a batch co-cultivation. Indeed, the continuous co-cultivation allows the establishment of bacterial interactions, the synchronization of growth rates and so the stabilisation of the consortium at a defined composition and/or profile. The inventors show that, if this particular step is replaced by batch cultivation, bacterial succession is observed in the culture instead of immediate interaction. Such succession leads to unfavourable conditions of certain bacterial groups and the underrepresentation of sensitive or slow grower bacterial strains in the composition and thus of particular decreased reproducibility and underrepresentation of certain functions in the consortium causing the destabilisation of the consortium. Therefore, in some preferred embodiments, the in vitro assembled consortium of selected bacterial strains comprises a plurality of functional groups fulfilling the above-mentioned criteria (a) and (b) during anaerobic co-cultivation.

Medium

The dispersing medium used in the method of the present invention of manufacturing the in vitro assembled consortia is added for a variety of reasons. First, the dispersing medium particularly ensures that bacteria remain as viable live bacteria. Further, the dispersing medium comprises nutrients and guarantees growth of the plurality of the selected bacterial strains representing all of the functional groups assembled into a particular consortium in the desired ratios. Still further, the dispersing medium plays an important role in recovery of the bacteria strains after storage. A broad range of solid or liquid dispersing media are known and may be used in the context of the present invention. Liquid media are used in particular for the anaerobic fermentation step III of the method of manufacture.

Suitable media include liquid media and solid supports. Liquid media generally comprise water and may thus also be termed aqueous media. Such liquid media may comprise a culture medium, a cryoprotective medium and/or a gel forming medium. Solid media may comprise a polymeric support.

Inoculation using diluted bacterial cultures are known in the field and include the use of preserved bacterial cultures. For establishment of the selected functional groups in an in vitro assembled consortium, the representative bacterial strains of each functional group are inoculated in concentrations reflecting their relative abundance of the respective function in the intestinal microbiome or in the targeted composition.

Cryoprotecting media are known in the field and include liquid compositions that allow freezing of bacteria strains essentially maintaining their viability. Suitable cryoprotecting agents may be identified by the skilled person, glycerol may be named by way of example. Inventive compositions comprising cryoprotecting agent are typically present as a suspension. Suitable amounts of cryoprotecting media may be determined by the skilled person in routine experiments; suitable are 5-50% v/v, preferably 10-40% v/v, such as 30% v/v. In one embodiment, the cryoprotecting medium comprises glycerol, preferably technical or industrial grade (i.e. comprising at least 95, 96, 97, 98 or 99% glycerol). Preferably, glycerol is present in 10, 20, 30, 40, 50 or 60% v/v in the cryopreserved inoculum and/or in the culture medium.

Lyophilisation is known in the field and include liquid compositions allowing to wash the bacterial strains maintaining their viability, for subsequent resuspension in lyophilisation buffer and subsequent lyophilisation.

Washing buffer may be identified by the skilled person, phosphate buffered saline (PBS) may be mentioned by way of example. Lyophilisation buffer may be identified by the skilled person as buffer solution containing sucrose, inulin, riboflavin, L-ascorbic acid and PBS. Suitable lyophilisation conditions may be determined by the skilled person in routine experiments.

Culture media are known in the field and include liquid compositions that allow the growth of bacteria strains. Typically, culture media include a carbon source (glucose, galactose, maltose, lactose, sucrose, fructose, cellobiose), “fibers” (preferably pectin, arabinogalac-tan, beta-glucan, soluble starch, resistant starch, fructo-oligosacharides, galacto-oligosacharides, xy-lan, arabinoxylans, cellulose), proteins (preferably yeast extract, casein, skimmed milk, peptone), co-factors (short chain fatty acids, hemin, FeSO4), vita-mins (preferably biotin, cobalamin, 4-aminobenzoic acid, folic acid, pyridoxamine hydrochloride), minerals (preferably sodium bicarbonate, potassium phosphate di-basic, potassium phosphate monobasic, sodium chloride, ammonium sulfate, magnesium sulfate, calcium chloride) and reducing agents (preferably cysteine, titanium(III)-citrate, yeast extract, sodium thioglycolate, dithiothreitol, sodium sulphide, hydrogen sulphite, ascorbate).

In particular, culture media include simple sugars carbon (glucose, galactose, maltose, lactose, sucrose, fructose, cellobiose), “fibers” (preferably pectin, arabinogalactan, beta-glucan, soluble starch, resistant starch, fructo-oligosacharides, galacto-oligosacharides, xylan, arabinoxylans, cellulose), proteins (preferably yeast extract, casein, skimmed milk, peptone), co-factors (short chain fatty acids, formate, lactate, succinate, hemin, FeSO4), vitamins (preferably biotin or D-(+)-Biotin (Vit. H), Cobalamin (Vit. B12), 4-aminobenzoic acid or p-aminobenzoic acid (PABA), folic acid (Vit. B11/B9), pyridoxamine hydrochloride (Vit. B6)), minerals (preferably sodium bicarbonate, potassium phosphate dibasic, potassium phosphate monobasic, sodium chloride, ammonium sulfate, magnesium sulfate, calcium chloride) and reducing agents (preferably cysteine, titanium(III)-citrate, yeast extract, sodium thioglycolate, dithiothreitol, sodium sulphide, hydrogen sulphite, ascorbate), guar gum, glycerol, potato starch, rice starch, pea starch, corn starch, wheat starch, inulin, succinate, formate, lactate, iron sulfate, tryptone, fucose, acetate, mucus, trehalose, mannitol, polysorbate and any combination thereof.

In one embodiment, the medium comprises intermediate metabolites, as an exogenous compounds, to allow an immediate growth of the intermediate utilizers. Preferably, the intermediate metabolites are one or more of lacate, succinate and formate.

In one embodiment, the culture medium comprises glycerol. Indeed, the inventors have shown that glycerol has a beneficial effect on butyrate production. Particularly, glycerol in the culture medium may serve as organic carbon source for bacteria, especially butyrate producers such as bacteria of functional group A2 and/or A6.

Cultivation methods and in particular also the handling and cultivating of anaerobic single strains are known and e.g. described by the Leibniz Institute DSMZ—German Collection of Microorganisms and Cell cultures available from the internet http://www.dsmz.de/catalogues/catalogue-microorganisms/culture-technology.html and regarding the cultivation of anaerobes in particular also http://www.dsmz.de/fileadmin/Bereiche/Microbiology/Dateien/Kultivierungshinweise/Anaerob.pdf For establishment of the selected functional groups in an in vitro assembled consortium, the representative bacterial strains of each functional group are inoculated in concentrations reflecting their relative abundance of the respective function in the intestinal microbiome or in the targeted composition. Exemplary ranges for functional groups in the inoculum are selected to include relative abundance of functional groups or bacterial strains of the functional groups (A1), (A2) and (A10) from 15-25%; functional group or bacterial strains of this functional group (A3) from 0.001-1%; functional group or bacterial strains of this functional group (A7) from 1-15%; functional groups or bacterial strains of the functional groups (A4), (A5), (A6), (A8) and (A9) from 5-25% (number of bacteria in comparison of the total number of bacteria, for instance as measured by 16S rRNA gene copies per ml of inoculum).

Composition

In one embodiment, the consortium of the invention is provided in the form of a composition or an inoculum. The invention then also relates to particular consortia, particular compositions comprising a consortium as disclosed herein and particular inocula comprising a consortium or a composition as disclosed herein.

The invention also relates to particular compositions comprising the consortium according to the invention, preferably the consortium such as obtained or obtainable by any method disclosed herein. In one embodiment, the composition comprises (i) viable bacterial strains and (ii) at least one end metabolite selected from the group consisting of acetate, propionate and butyrate, and mixtures thereof, wherein the composition comprises a combination of bacterial strains as specifically disclosed above, and wherein the composition comprises at least 10⁹ bacterial cells per ml or μg for each bacterial strain; and wherein each of the bacterial strains has a viability over 25%, 30%, 40%, 50%, preferably over 70%. In one embodiment, at least 20 μg of viable bacterial cells are obtained from 1 mL of composition, for example after lyophilization. The viable bacterial strains are combination of bacteria strains or consortium as disclosed herein.

The following formula is used to describe the biomass of a bacterial culture. The formula is dependent on the geometric form of the (bacterial) cell and thus varies for each consortium:

For cocci the equation for the biovolume (Bv) is:

$$Bv = \frac{π}{4} {W}^{3} ( L - W * 3 ) ,$$

whereby D stands for diameter.

For rod shaped bacteria the equation is:

$$Bv = \frac{π}{6} {D}^{3}$$

whereby W stands for width and L for length.

The following equation permits to obtain the biomass of a population of cells:

Biomass [μg/mL]=N[number of cells/mL]*Bv [μm³]*F [μg/m³]

Where:

N=number of organisms per ml of sample examined,

Bv=biovolume obtained as described above,

F=conversion factor (quantity of carbon by cellular volume). F is strain specific and has been reported for a multitude of strains in literature, where values of F for pure cultures.

In one aspect, the composition comprises at least 10⁶, 10⁷, 10⁸, 10⁹, bacterial cells per μg of dry composition, preferably between 10⁸ and 10⁹, bacterial cells per μg of composition.

In a particular aspect, the composition is such that it does not comprise a bacterium from the genus Blautia, nor an archaea of the genus Methanobrevibacter or Methanomassiliicoccus, especially Blautia hydrogenotrophica, Blautia producta, Methanobrevibacter smithii and Candidatus Methanomassiliicoccus intestinalis, particularly when the composition comprises Eubacterium limosum, particularly when the composition comprises Eubacterium limosum such as to fulfils the metabolic function of functional group A9, preferably A9 and A6.

In a particular aspect, the composition is such that it does not comprise a bacterium from the genus Blautia, Acetobacterium, Clostridium, Moorella, and Sporomusa, nor an archaea of the genus Methanobrevibacter or Methanomassiliicoccus, especially Acetobacterium carbinolicum, Acetobacterium malicum, Acetobacterium wieringae, Blautia hydrogenotrophica, Blautia producta, Clostridium aceticum, Clostridium glycolicum, Clostridium magnum, Clostridium mayombe, Methanobrevibacter smithii and Candidatus Methanomassiliicoccus intestinalis, particularly when the composition comprises Eubacterium limosum, particularly when the composition comprises Eubacterium limosum such as to fulfils the metabolic function of functional group A9, preferably A9 and A6.

In a particular aspect, preferably when the composition comprises an Eubacterium, preferably Eubacterium limosum, the composition is such that it does not comprise Blautia hydrogenotrophica.

In another particular aspect, the composition is such that it does not comprise a bacterium from the genus Blautia, especially Blautia hydrogenotrophica and/or Blautia producta, particularly when the composition comprises an Eubacterium, preferably Eubacterium limosum.

Additionally or alternatively, particularly when the composition comprises an Eubacterium, preferably Eubacterium limosum, the composition is such that it does not comprise:
an archaea of the genus Methanobrevibacter or Methanomassiliicoccus, preferably Methanobrevibacter smithii and/or Candidatus Methanomassiliicoccus intestinalis,
a bacterium of the genera Acetobacterium, preferably Acetobacterium carbinolicum, Acetobacterium malicum and/or Acetobacterium wieringae,
a bacterium of the genera Moorella and/or Sporomusa; and/or
a bacterium selected from Clostridium aceticum, Clostridium glycolicum, Clostridium magnum and/or Clostridium mayombe.

In another particular aspect, the present invention relates to a composition comprising a consortium as detailed above comprising Enterococcusfaecalis.

In another particular aspect, the present invention relates to a composition comprising a consortium as detailed above comprising Roseburia hominis.

In another particular aspect, the present invention relates to a composition comprising a consortium as detailed above comprising Eubacterium limosum and Roseburia hominis; Eubacterium limosum and Enterococcusfaecalis; Eubacterium limosum, Roseburia hominis and Enterococcusfaecalis.

Preferably, the composition according to the invention is free of, or essentially free of, other viable, live bacteria (i.e., other than the bacterial strains of the consortium).

Particularly, the composition according to the invention is free of, or essentially free of intermediate metabolites, preferably selected from the group consisting of succinate, formate and lactate.

In one embodiment, the composition further comprises dispersing medium. Alternatively, the composition may be free of, or essentially free of dispersing medium.

In one embodiment, the consortium of the invention is provided in the form of an inoculum. Any particular composition disclosed hereabove can then be comprised in the inoculum according to the invention.

Preferably, the inoculum comprises a sufficient amount of the bacterial strains to achieve a concentration of 10³ to 10¹⁴ 16S rRNA gene copies per ml of the culture-suspension as quantified by qPCR in the bioreactor after addition to the bioreactor. In particular, this concentration is for each bacterial strains of the consortium.

Particularly, the consortium is provided as an inoculum in step I of the method according to the invention. In one embodiment, the consortium is provided in the form of a preserved inoculum, preferably by a cryopreservation method or a sample preserved by lyophilization. In a preferred embodiment, the inoculum is cryopreserved with glycerol.

The provision of a preserved sample, in particular a cryopreserved or lyophilized sample, as inoculum surprisingly has the advantages 1) that the time period of anaerobic co-cultivation required until the microbial and metabolic profiles stabilize is significantly reduced, (e.g. reduced by a factor of 2 or 3, preferably compared to a fresh inoculum) and 2) that the use of preserved samples greatly simplifies standardization and quality control of the manufacturing process and manufactured products such as to fulfil required good manufacturing practice standards and inter-batch comparability, in particular in the pharmaceutical industry.

Pharmaceutical Composition

In some embodiments of the method of manufacturing an in vitro assembled consortium the method comprises post-treatment steps or one or more further processing steps for providing the in vitro assembled consortium as a pharmaceutical composition. Such pharmaceutical compositions may be formulated according to known principles and adapted to various modes of administration. In one embodiment, the inventive pharmaceutical compositions are adapted to rectal administration. In one further embodiment, the inventive pharmaceutical compositions are adapted to oral administration.

In some embodiments the method of manufacturing an in vitro assembled consortium and in some embodiments of the method of providing an in vitro assembled consortium the method comprises assembling consortia adapted for therapeutic use or personalized medicine, thereby targeting diseases with associated microbiota dysbiosis to specific patient groups or individuals. Bacteria showing similar functionalities but different taxonomic identities can be replaced and exchanged in the in vitro assembled consortium used for treatment according to the loss of bacteria detected in patients or specific indications. Loss in phylogenetic diversity and functionality can be targeted for the first time, since the consortium approach allows the controlled re-establishment of single niches in the patient's gut. For example, the engraftment of a formate producing Bifidobacterium will be guaranteed by the combination with the formate-utilizing strain such as Blautia strain in order to avoid enrichment of the intermediate metabolite, that would lead to the elimination of both strains.

In one preferred embodiment, the pharmaceutical composition comprises the consortium as obtained or produced by any method disclosed herein, particularly after step IV or V. Alternatively, the pharmaceutical composition comprises an inoculum of the consortium, for example such as provided in step I of the methods according to the invention.

The pharmaceutical compositions of the invention can additionally comprise any pharmaceutically acceptable carriers known in the art.

In one embodiment, the pharmaceutical composition is to be administered orally. For oral administration, the pharmaceutical or veterinary composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

Well-known thickening agents may also be added to compositions such as corn starch, agar, natural or synthetic gums, resins, methylcellulose, sodium carboxymethylcellulose, guar, xanthan and the like. Preservatives may also be included in the composition, including methylparaben, propylparaben, benzyl alcohol and ethylene diamine tetraacetate salts.

Pharmaceutical or veterinary compositions according to the invention may be formulated to release the active ingredients substantially immediately upon administration or at any predetermined time or time period after administration.

In one embodiment, the pharmaceutical composition further comprises prebiotics. Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-1), dietary fibers (e.g., soy fiber, sugarbeet fiber, pea fiber, corn bran, and oat fiber) and xylooligosaccharides.

In one embodiment, the pharmaceutical composition is to be administered in a transmucosal way. For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Preferably, the composition is in a gastro-resistant oral form allowing the bacteria contained in the composition, and more particularly the consortium according to the invention, to pass the stomach and be released into the intestine. Alternatively, the enteric material is acid stable and labile at basic pH, which means that it does not dissolve in the stomach, but dissolves in the intestine. The material that can be used in enteric coatings includes, for example, alginic acid, cellulose acetate phthalate, plastics, waxes, shellac and fatty acids (e.g. stearic acid or palmitic acid).

The composition of the excipient or carrier can be modified as long as it does not significantly interfere with the pharmacological activity of the consortium according to the invention.

Preferably, the pharmaceutical composition an effective therapeutic amount of the consortium according to the invention, preferably 10³ to 10¹⁴ CFU (colony forming units) of bacteria per ml or μg of the pharmaceutical composition.

Optionally, the pharmaceutical composition may further comprise an additional active ingredient, for instance an anti-inflammatory agent, an immuno-suppressive agent or an anti-cancer agent.

Use

The invention also relates to the use of the consortium or of the pharmaceutical composition as a medicament, especially in the treatment of a disorder or disease, in particular caused or resulted in dysbiosis. Then, the invention also relates to a method for treating a disorder or a disease comprising the administration of a therapeutically effective amount of the pharmaceutical composition or the consortium according to the invention. It also relates to a composition or a consortium as disclosed herein for use for treating a disease and to the use of a composition or a consortium as disclosed herein for the manufacture of a medicament for treating a disease.

The pharmaceutical compositions may find use in a number of indications. Thus, the invention provides for pharmaceutical compositions as described herein for use in the prophylaxis, treatment, prevention or delay of progression of a disease related to intestinal microbiome dysbalance or associated with microbiota dysbiosis. It is generally accepted that dysbiosis originates from an ecological dysbalance (e.g. based on trophism), characterized by disproportionate amounts or absence of bacteria strains in the microbiome of the patient which are essential for the establishment and/or maintenance of a healthy microbiome. In one embodiment, such a disease or disorder is selected from intestinal infections, including gastro-intestinal cancer, colorectal cancer (CRC), auto-immune disease, infections such as caused by virus or bacteria, ulcers, gastroenteritis, Guillain-Barre syndrome, graft versus host disease (GvHD), gingivitis and nosocomial infection. In particular, the disease can be selected from Clostridium difficile infection (CDI), vancomycin resistant enterococci (VRE), post-infectious diarrhea, inflammatory bowel diseases (IBD), including ulcerative colitis (UC) and Crohn's disease (CD). The inventive pharmaceutical compositions are particularly suited for treatment of IBD and CDI.

Preferably, the disease or disorder to be treated is selected from the group consisting of Clostridium difficile infection (CDI), vancomycin resistant enterococci (VRE), post-infectious diarrhea, inflammatory bowel diseases (IBD), including ulcerative colitis (UC) and Crohn's disease (CD), colorectal cancer (CRC), allo-HSCT associated diseases or Graft versus Host Disease (GvHD).

In a particular embodiment, the invention concerns a consortium or a pharmaceutical composition for use in the treatment of pathologies involving bacteria of the human microbiome, preferably the intestinal microbiome, such as inflammatory or auto-immune diseases, cancers, infections or brain disorders.

Indeed, some bacteria of the microbiome, without triggering any infection, can secrete molecules that will induce and/or enhance inflammatory or auto-immune diseases or cancer development.

Therefore, a further object of the invention is a method for controlling the microbiome of a subject, preferably the intestinal microbiome, comprising administering an effective amount of the pharmaceutical composition or consortium as disclosed herein in a subject.

In one embodiment, the medicament or pharmaceutical composition can be used in combination with an anti-inflammatory agent, one or more immuno-suppressive or anti-cancer agents. Such immuno-suppressive agents may be glucocorticoids, cytostatics or antibodies. Such anti-cancer agents may be chemotherapy or radiotherapy agents, for example drugs, hormones or antibodies.

Novel modalities applied in microbiome therapies such as therapies using phage, or phage like particles, DNA modifying, transferring or transcription silencing techniques and genetically modified bacteria can be used in combination with the composition of this invention.

The subject to treat according to the invention is an animal, preferably a mammal, even more preferably a human. However, the term “subject” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, or non-mammals such as poultry, that are in need of treatment. Preferably, the subject is a human.

In a particular embodiment, the subject has already received at least one line of treatment, preferably several lines of treatment, prior to the administration of the consortium or the pharmaceutical composition according to the invention.

Preferably, the treatment is administered to the subject regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

Physiological data of the patient or subject (e.g. age, size, and weight) and the routes of administration have to be taken into account to determine the appropriate dosage, so as a therapeutically effective amount will be administered to the patient or subject.

### Aspects of the Invention

Various aspects and embodiments of the invention are also described in the clauses No. 1 to 16 listed below:

1. A method of manufacturing an in vitro assembled consortium of selected live, viable bacterial strains by an anaerobic co-cultivation in a dispersing medium,

wherein the consortium comprises a plurality of functional groups each group comprising at least one of the selected bacterial strains,

wherein each functional group of selected bacterial strains performs at least one metabolic pathway of an anaerobic microbiome, in particular of an intestinal microbiome,

wherein the method of manufacturing comprises the steps of

I. providing a sample of the assembled consortium as an inoculum,

wherein in particular the sample of the consortium is obtained from a prior continuous anaerobic co-cultivation process of the selected bacterial strains until a stable microbial profile and a stable metabolic profile characteristic of the in vitro assembled consortium has been established, and/or wherein in particular the sample is obtained as a preserved sample;

II. adding the inoculum to the dispersing medium in a bioreactor thereby forming a culture-suspension of the selected bacterial strains;

III. multiplying the selected bacterial strains in the culture suspension by co-cultivation until a stable microbial profile and a stable metabolic profile characteristic of the in vitro assembled consortium is established;

IV. harvesting the consortium of the selected live, viable bacterial strains;

V. optionally, subjecting the harvested consortium to one or more post-treatment steps; characterized in that step III is performed in an anaerobic batch fermentation process or in an anaerobic fed-batch fermentation process.

2. The method of manufacturing according to claim 1,

wherein the dispersing medium comprises selected nutrients comprising starches, fibers and proteins;

wherein in step III at least one of the criteria (a), (b), (c), (d) is fulfilled, wherein:

according to criteria (a) the selected bacterial strains perform a degradation of the selected nutrients directly, or indirectly via an intermediate metabolite, to a short chain fatty acid, in particular to one or more of acetate, propionate and butyrate;

according to criteria (b) the plurality of functional groups enables metabolic cross-feeding interactions during co-cultivation by comprising a functional group which produces a particular intermediate metabolite and by comprising a functional group consuming said intermediate metabolite, said intermediate metabolite selected from formate, lactate and succinate;

according to criteria (c) a concentration in the culture-suspension of any intermediate metabolite produced during the degradation is below the concentration inhibiting proliferation of all bacterial strains provided in one of the functional groups;

wherein in particular the intermediate metabolite is selected from formate, lactate and succinate;

according to criteria (d) a concentration in the culture-suspension of one or more inhibitory compound produced as a by-product of the degradation, in particular H₂, or a concentration in the culture-suspension of environmental O₂, is below the concentration inhibiting proliferation of all bacterial strains provided in one of the functional groups;

wherein, in particular, criteria (a) and (b) are fulfilled or wherein more particularly criteria (a), (b) and (c) are fulfilled or criteria (a), (b) and (d) are fulfilled or criteria (a), (b) (c) and (d) are fulfilled.

3. The method of manufacturing according to claim 1 or 2, wherein the stable microbial profile of the in vitro assembled consortium exhibits an abundance of each of the selected bacterial strains in the consortium of 10⁵-10¹⁴ 16S rRNA gene copies per ml of the culture suspension, and wherein the stable metabolic profile of the in vitro assembled consortium provided as inoculum in step 1 and at the time of harvest in step 4 fulfils one or more of the following criteria:
a concentration of one or more of the intermediate metabolites formate, lactate, succinate in the dispersing medium are each below 15 mM, in particular below 10 mM, 5 mM, 1 mm or more particular below 0.1 mM.
a concentration of one or more of propionate and butyrate are above 5 mM, in particular above 10 mM, more particular above 15 mM and wherein the concentration of acetate is above 10 mM, in particular above 20 mM, more particular above 40 mM.

4. The method according to any one of the previous claims, wherein the sample of the consortium of step 1 is selected from a preserved sample preserved by a cryopreservation method or a sample preserved by lyophilisation.

5. The method according to any one of the previous claims, wherein the inoculum of step 1 comprises a sufficient amount of the bacterial strains to achieve a concentration of 10³ to 10¹⁴ 16S rRNA gene copies per ml of the culture-suspension as quantified by qPCR in the bioreactor after addition to the bioreactor in step II and prior to step III.

6. The method according to any one of the previous claims, wherein step 3 is performed as a fed-batch fermentation process comprising two or more sub-steps of batch cultivation, in particular for a duration of 12 up to 24 or up to 48 hours,

wherein between each of the sub-steps a further portion of a dispersing medium providing one or more of the complex compounds, selected from sugars, starches, fibers and proteins is added to the bioreactor and wherein in particular step 3 is performed as a two-step fed-batch fermentation process comprising the steps of:

III-1 batch fermentation for the duration of one day, in particular for 24 hours, with a dilution of the inoculum into the dispersing medium ranging from 1% to 20% of inoculum to dispersing medium (v/v);

III-2 addition of dispersing medium, in particular addition of a volume of dispersing equal to the volume of the culture-suspension in the bioreactor

III-3 continuation of the fermentation for another day, in particular for a further 24 hours.

7. The method according to any one of the previous claims, wherein during step III or prior to step IV one or more parameter regarding the microbial profile and/or regarding the metabolic profile of the culture suspension is measured,

wherein optionally the measured value of the one or more parameter is compared to a standard value of said one or more parameter and

wherein the standard value of said one or more parameter corresponds to the value as measured in a culture-suspension comprising the dispersing medium and the selected bacterial strains grown in an anaerobic continuous co-cultivation until said measured value has stabilized over a period of at least 3 days, in particular at least 5 or 7 days.

8. The method according to claim 9, wherein the standard value of the one or more parameter corresponds to a standard value as indicated below:
a concentration of succinate below 15 mM, 10 mM, 5 mM, 1 mM or 0.1 mM
a concentration of formate below 15 mM, 10 mM, 5 mM, 1 mM or 0.1 mM
a concentration of lactate below 15 mM, 10 mM, 5 mM, 1 mM or 0.1 mM
a concentration of acetate above 10 mM, 20 mM or 40 mM
a concentration of propionate above 5 mM, 10 mM or 15 mM
a concentration of butyrate above 5 mM, 10 mM or 15 mM
a redox value below −300 mV, −350 mV or −400 mV,
an optical density above 1.5, 2 or 3
a viability of over 50%, 60% or 70%
an abundance of bacterial strains of 10⁵-10¹⁴ 16S rRNA gene copies per ml

9. The method according to any one of the previous claims, wherein a sample of the consortium harvested in step 4 is used directly or is preserved and subsequently used as the inoculum of step 1 in another round of performing the method according to one of the previous claims.

10. The method according to any one of the previous claims comprising an additional preparatory stage prior to step 1,

wherein in the preparatory stage the inoculum of step 1 comprising the consortium of the selected viable, live bacterial strains is manufactured from a single-strain sample of each of the selected bacterial strains,

wherein said preparatory stage comprises the steps of:

(a) providing single strain samples of the selected viable, live bacterial strains,

(b) inoculating the selected strains into the dispersing medium in a bioreactor thereby forming a culture suspension and co-cultivating the culture suspension in an anaerobic continuous co-cultivation,

(c) harvesting the consortium of the bacterial strains from the bioreactor after the culture-suspension has established a stable microbial profile and a stable metabolic profile,

(d) optionally subjecting the harvested consortium of the bacterial strains to post-treatment steps.

11. The method according to claim 10, wherein step (a) of the preparatory stage comprises the steps of:

(a1) providing and separately cultivating said single strain samples in the presence of a substrate specific for each of said strains thereby obtaining single-strain cultures,

(a2) combining said single-strain cultures of (a1) into a culture-suspension and co-cultivating them under anaerobic conditions in the presence of a dispersing medium,

wherein in particular, the dispersing comprises nutrients selected from pectin, arabinogalactan, beta-glucan, soluble starch, resistant starch, fructo-oligosacharides, galacto-oligosacharides, xylan, arabinoxylans, cellulose, yeast extract, casein, skimmed milk, peptone wherein in particular a pH value is adjusted within a range of pH 5-7, more particularly a range of pH 5.5-6.5 and

wherein in particular after a duration of 1 or 2 days of co-cultivation half of the volume of the culture-suspension is replaced by the same volume of fresh dispersing medium,

and wherein step (a2) is terminated once metabolites succinate, formate and lactate are each below 15 mM.

12. The method according to any one of the previous claims wherein in one or both of the optional steps selected from step 5 of anyone of claims 1 to 11 and step d) of any one of claims 10 to 11, the harvested culture-suspension comprising the consortium of the selected bacterial strains is subjected to a preservation-treatment,

wherein the culture-suspension harvested from the bioreactor is handled and stored under protection from oxygen,

wherein the preservation-treatment is selected from cryopreservation and lyophilisation,

wherein the post-treatment of cryopreservation comprises the steps of:
mixing the harvested culture-suspension with a cryoprotective solution in particular obtaining a 1:1 (v/v) mixture of culture-suspension and glycerol or
centrifuging the harvested culture-suspension and resuspending an obtained pellet in a mixture of the cryoprotective solution and the dispersing medium, in particular in a 1:1 (v/v) mixture of glycerol and the dispersing medium
shock freezing with liquid N₂ or gradually freeze to a storage temperature of at least −20° C., in particular at 20° C. to −80° C.,

wherein the post-treatment of lyophilisation comprises the steps of:
centrifuging the harvested culture-suspension and wash an obtained pellet with a buffer solution
resuspending the pellet in a lyophilisation solution and lyophilise
subsequent storage at a temperature of 4° C. or lower.

13. The method according to one of claims 1 to 8 wherein the sample of the consortium provided as inoculum in step I is a preserved sample of the consortium preserved according to the preservation treatment of claim 14,

wherein a cryopreserved sample of the consortium is thawed at room temperature and inoculated into the bioreactor with an inoculation ratio of 0.1-25% (v/v), in particular with a 0.5-2% (v/v); or

wherein a lyophilised sample of a culture suspension is re-suspended in the dispersing medium and inoculated into the bioreactor with an inoculation ratio of 0.1-25% (v/v), in particular 0.5-2% (v/v); and

wherein the total amount of the selected bacterial strains added to the bioreactor in step 11 provides for a concentration of 10³-10¹⁴ 16S rRNA gene copies as quantified by qPCR per ml of the culture suspension in the bioreactor prior to step III.

14. A method of providing an in vitro assembled consortium of selected live, viable bacterial strains, wherein the consortium comprises a plurality of functional groups comprising a subset of functional groups A1 to A9,

or wherein the plurality of functional comprises A1 to A10 or subsets thereof, and wherein functional groups A1 to A10 are:
(A1) Resistant starch degraders;
(A2) Starch degrading-, acetate-consuming butyrate-producers;
(A3) Oxygen-reducing lactate- and formate-producers;
(A4) Starch-reducing lactate- and formate-producers;
(A5) Protein- and lactate-utilizing propionate-producers;
(A6) Starch-, protein- and lactate-utilizing butyrate-producers;
(A7) Starch- and protein-degrading formate- and lactate-producers;
(A8) Protein-, succinate-utilizing, propionate-producers;
(A9) Hydrogen- and formate-utilizing acetate-producers;
(A10) is an additional functional group of succinate producers.

15. A composition comprising an in vitro assembled consortium of selected live, viable bacterial strains, wherein the consortium is obtainable according to the method of claim 14.

16. The method according to one of claims 1 to 13,

wherein the in vitro assembled consortium provided as inoculum in step 1 of any one of claims 1 to 13 or in step (a) of claim 10 or 11 is assembled according to the method of claim 14.

### EXAMPLES

To further illustrate the invention, the following examples are provided. These examples are provided with no intend to limit the scope of the invention.

### Example 1: Rationale, Functional Groups

Bacterial strains were isolated from healthy donors using Hungate anaerobic culturing techniques (Bryant, 1972) and characterized for growth and metabolite production on M2GSC Medium (ATCC Medium 2857) and modifications thereof whereby the carbon sources glucose, cellobiose and starch were replaced by specific substrates including intermediate metabolites and fibers found in the human intestine. The concentrations of the produced metabolites were quantified by refractive index detection HPLC (Thermo Scientific Accela™, ThermoFisher Scientific; HPLC-RI). HPLC-RI analysis was performed using a SecurityGuard Cartridges Carbo-H (4×3.0 mm) (Phenomenex, Torrence, USA) as guard-column connected to a Rezex ROA-Organic Acid H+ column (300×7.8 mm) (Phenomenex). Bacteria cultures to be analyzed were centrifuged at 14.000-x g for 10 min at 4° C. Filter-sterilized (0.45 μL) supernatants were analyzed. Injection volume for each sample was 40 μL. HPLC-RI was run at 40° C. with a flow rate of 0.4 mL/min and using H2SO4 (10 mM) as eluent. Peaks were analyzed using AgilentEzChrome Elite software (Version: 3.3.2 SP2, Agilent Technologies, Inc. Pleasanton, USA). Clusters were formed based on substrate usage and metabolite production. Functional groups were defined as combinations of substrate-utilization and metabolite-production as described in claim 1. Nine strains were selected within those clusters in order to assemble the core intestinal carbohydrate metabolism and result in an exclusive production of end metabolites (acetate, propionate and butyrate), without accumulation of intermediate metabolites (formate, succinate, lactate).

As outlined above, the combination of functional groups represented by one or more bacteria strains as disclosed herein is chosen to:
Degrade the main energy sources in the gut including fibers and intermediate metabolites (all groups, A1-A10)
Protect anaerobiosis by reduction of the eventual 02 through respiration (A3)
Produce the main end metabolites found in the intestine (A1, A2, A3, A4, A5, A9, A10)
Prevent the enrichment of intermediate metabolites (A5, A6, A7, A8, A9) independent of the composition of the recipient's microbiome.

For group (A1), Ruminococcus bromii was cultivated in YCFA medium (Duncan, Hold, Harmsen, Stewart, & Flint, 2002) for 48 hours using the Hungate technique (Bryant, 1972) resulting in the production of formate (>15 mM) and acetate (>10 mM) as quantified by HPLC-RI.

For group (A2), Faecalibacterium prausnitzii was cultivated in M2GSC medium (ATCC Medium 2857) for 48 hours using the Hungate technique (Bryant, 1972) resulting in the consumption of acetate (>10 mM) and in the production of formate (>20 mM) and butyrate (>15 mM) as quantified by HPLC-RI.

For group (A3), Lactobacillus rhamnosus was cultivated in MRS Broth (Oxoid) for 48 hours using the Hungate technique (Bryant, 1972) resulting in the production of lactate (>50 mM) and formate (>10 mM) as quantified by HPLC-RI.

For group (A4), Bifidobacterium adolescentis was cultivated in YCFA medium (Duncan et a1., 2002) for 48 hours using the Hungate technique (Bryant, 1972) resulting in the production of acetate (>50 mM), formate (>15 mM) and lactate (>5 mM) as quantified by HPLC-RI.

For group (A5), Clostridium (Anaerotignum) lactatifermentans was cultivated in modified M2-based medium (ATCC Medium 2857) supplemented with DL-lactate [60 mM] instead of a carbohydrate source for 48 hours using the Hungate technique resulting in the consumption of lactate (at least 10 mM) and in the production of propionate (>30 mM), acetate (>10 mM) as detected by HPLC-RI.

For group (A6), Eubacterium limosum was cultivated in YCFA medium (Duncan et a1., 2002) for 48 hours using the Hungate technique (Bryant, 1972) resulting in the production of acetate (>10 mM) and butyrate (>5 mM) as quantified by HPLC-RI.

For group (A7), Collinsella aerofaciens was cultivated in YCFA medium (Duncan et a1., 2002) for 48 hours using the Hungate technique resulting in the production of formate (>20 mM), lactate (>15 mM) and acetate (>15 mM) as quantified by HPLC-RI.

For group (A8), Phascolarctobacterium faecium was cultivated in M2-based medium (ATCC Medium 2857) supplemented with succinate (60 mM) as sole carbohydrate source for 48 hours using the Hungate technique (Bryant, 1972) resulting in the full consumption of succinate (60 mM) and in the production of propionate (60 mM) as quantified by HPLC-RI.

For group (A9), Blautia hydrogenotrophica was cultivated in anaerobic AC21 medium (Leclerc, Bernalier, Donadille, & Lelait, 1997) for >75 hours using the Balch type tubes resulting in the production of acetate (>20 mM) as quantified by HPLC-RI, and consumption of hydrogen.

For group (A10), B. fragilis was cultivated in was cultivated in YCFA medium (Duncan, Hold, Harmsen, Stewart, & Flint, 2002) for 48 hours using the Hungate technique (Bryant, 1972) resulting in the production of succinate (>20 mM) and acetate (>10 mM) as quantified by HPLC-RI.

The combination of strains from the functional groups (A1)-(A10) encompass key functions of the microbiome and results, if cultured together, in a trophic chain analog to the healthy intestinal microbiome in its capacity to exclusively produce end metabolites from complex carbohydrates without accumulation of intermediate metabolites.

### Example 2: In Vitro Assembly of Consortium

In order to establish the exemplary consortium consisting of 9 functional groups A1-A9 using one stains from each functional group forth on named PB002 in a growing and metabolically interacting manner, a previously validated model for anaerobic intestinal fermentations (Zihler et al., 2013) was adapted using a simplified medium based on YCFA (DSMZ Media N^{o} 1611). Thereby, the 5 g/L of glucose that are the carbon source in YCFA were replaced by 2 g/L of pectin (Sigma Aldrich), 1 g/L of fructo-oligosacharaides (FB97, Cosucra), 3 g/L of potato starch (Sigma Aldrich), and 2 g/L of corn starch (Sigma Aldrich). A 200 ml bioreactor (Infors HT) was inoculated with a mix of overnight cultures of all 9 strains and inoculated anaerobically at a 1/100 dilution.

The bioreactor was consecutively operated at pH 6.5 for 24 h in order to allow growth of primary degraders and subsequent consumption of the produced intermediate metabolites. Growth was monitored by base consumption and optical density. Metabolites were monitored using HPLC-RI as described above. After the first batch-fermentation, new medium was fed by removing half of total volume and refilling with medium to the original volume of 200 ml in the bioreactor. After the second batch fermentation cycle the metabolic profile did not contain any intermediate metabolites and >40 mM acetate and >5 mM of propionate and butyrate each. From the end of the second batch fermentation on, the bioreactor was operated continuously at a volume of 200 ml, a flow rate of 12.5 ml/h and a pH of 6.5. Subsequently, a stable metabolic profile established within 7 days after inoculation containing exclusively the desired end metabolites of acetate, propionate and butyrate without detection of intermediate metabolites showing constant production of all desired metabolites without washout of any functional group.

PB002 could therefore be cultivated in a bioreactor and showed the desired properties based on key functional groups defined of the intestinal microbiome defined in FIG. 1, i.e. degradation of fibers and proteins into exclusively end-metabolites, a clear indication that the desired interactions and metabolic activities defined in (A1)-(A9) were established in a continuously operated bioreactor.

### Example 3: Quantification of Bacterial Strains in the In Vitro Assembled Consortium

To test maintenance of all 9 bacterial strains of exemplary consortium PBTG2 in the bioreactor over time qPCR quantification of the single strains of the consortium was performed using the primers listed in table 2.

TABLE 2

| Group *) | Bacteria strains | Primer FW 5′-3′ | Primer RV 5′-3′ |
|---|---|---|---|
| A1 ¹⁾ | Ruminococcus | CGCGT GAAGG ATGAA | TCAGT TAAAG CCCAG |
| | bromii | GGTTT TC | CAGGC |
| A2 ¹⁾ | Faecalibacterium | CGCGG TAAAA CGTAG | CTGGG ACGTT GTTTC |
| | prausnitzii | GTCAC A | TGAGT TT |
| A3 ¹⁾ | Lactobacillus | GGAAT CTTCC ACAAT | CATGG AGTTC CACTG |
| | rhamnosus | GGACG CA | TCCTC TT |
| A4 ¹⁾ | Bifidobacterium | GTCCATCG CTTAACGG | ACCAC CTGTG AACCC |
| | adolescentis | TGGATC | GC |
| A5 ¹⁾ | Clostridium | GCACT CCACC TGGGG | CAACC TTCCT CCGGG |
| | (Anaerotignum) | AGT | TTATC CA |
| | lactatifermentans | | |
| A6 ²⁾ | Eubacterium | GGCTT GCTGG ACAAA | CTAGG CTCGT CAGAA |
| | limosum | TACTG | GGATG |
| A7 ¹⁾ | Collinsella | GGTAG GGGAG GGTGG | GCGGT CCCGC GTGGG |
| | aerofaciens | AAC | TT |
| A8 ¹⁾ | Phascolarcto- | GGAGT GCTAA TACCG | CCGTG GCTTC CTCGT |
| | bacterium faecium | GATGT GA | TTACT |
| A9 ¹⁾ | Blautia | CGTGA AGGAA GAAGT | TCAGT TACCG TCCAG |
| | hydrogenotrophica | ATCTC GGTA | CAGGC C |
| A1-A9 ³⁾ | All bacteria | GTGST GCAYG GYTGT | ACGTC RTCCC CRCCT |
| | | CGTCA | TCCTC |

*) sources: ¹⁾DECIPHER database; ²⁾Wang et al. (1996), ³⁾Maeda et al., (2003)

DNA from pellets of the fermentation effluent was extracted using the FastDNA™ SPIN Kit for Soil (MP Bio). Genomic DNA extracts were 50-fold diluted using DNA-free H₂O. qPCRs were performed using Mastermix SYBR® green 2× and LowRox (Kapa Biosystems), primers (10 μM) and DNA-free H₂O were used in a ABI 7500 FAST thermal cycler (Applied Biosystems) as recommended by the producer and quantified using standards of amplified whole 16S rRNA gene amplicon sequences of the strains used for the consortium cloned into the pGEMT easy vector (Promega, Madison Wis., USA). Amplification of the whole 16S rRNA gene was performed with a combination of whole 16S rRNA gene amplification primers using one forward and one reverse primer of the primers listed in Table 3. qPCR quantification of the single strains is shown in copies of genomic 16S rRNA gene per ml of culture in FIG. 5.

TABLE 3

| | | Orientation of |
|---|---|---|
| | | the Primer on |
| | | 16S rRNA Gene |
| Name *) | Sequence 5′-3′ **) | Sequence 5′-3′ |
| 518R ⁵⁾ | ATTAC CGCGG CTGCT GG | Reverse |
| 1392R ¹⁾ | ACGGG CGGTG TGTRC | Reverse |
| 1412R ²⁾ | CGGGT GCTNC CCACT TTCAT G | Reverse |
| 1492R ⁴⁾ | GNTAC CTTGT TACGA CTT | Reverse |
| 1492R.E ¹⁾ | TACGG YTACC TTGTT ACGAC TT | Reverse |
| 1525R ¹⁾ | AAGGA GGTGW TCCAR CC | Reverse |
| F8 ⁴⁾ | AGAGT TTGAT CMTGG CTC | Forward |
| F15 ²⁾ | GATTC TGGCT CAGGA TGAAC G | Forward |
| F27 ¹⁾ | AGAGT TTGAT CMTGG CTCAG | Forward |
| F518 ⁵⁾ | CCAGC AGCCG CGGTA ATACG | Forward |

*) sources: ¹⁾Lane, 1991, ²⁾Kaufmann et al., 1997, ⁴⁾Mosoni et al., 2007), ⁵⁾Muyzer et al., 1993 **) nucleic codes as defined in IUPAC nucleotide code, particularly: N = any base, R = A or G.

### Example 4: Viability of Consortium

To quantify the total amount of viable cells in the bioreactor, effluent was analyzed using the sybr green, propidium iodide method whereby living cells are stained by sybr green and dead cells by propidium iodine and sybr green allowing the quantification of total viable and dead cells were counted with flow cytometry on 4 consecutive days of fermentation using a Beckman Coulter Cytomics FC 500. Absolute counts were determined with Beckman Coulter Flow-Count Fluorospheres. Cell count in the bioreactor reached over 10¹⁰ viable bacterial cells per ml of culture with a viability of >90%.

It followed that co-culturing allows high density, high viability culturing under continuous fermentation at a retention time of 16h.

### Example 5: Preservation of In Vitro Assembled Consortia

To store the described exemplary consortium PB002 and to compare different stabilization techniques and their impact on the stability of PB002, the effluent of the consortium of PB002 continuously fermented for at least 7 days was processed in using the following procedures:
Effluent was anaerobically mixed 1:1 with an anaerobic cryoprotective medium containing 60% glycerol and 40% of the dispersing medium previously described in example 2. The cryoprotected formulation was snap cryopreserved using liquid nitrogen and stored at −20° C. for at least 3 months.
Effluent was centrifuged for 4 min at 3.500×g at RT, pellet washed in phosphate buffered saline (PBS) and centrifuged again as described before. Pellet was resuspended 1:20 in lyophilisation buffer solution containing sucrose, inulin, riboflavin, L-ascorbic acid and PBS. Aliquots were lyophilised and stored at +4° C. for at least 3 months.

The stored effluents were used to initiate a continuous fermentation as described in example 2. All stabilization techniques showed viability of all bacteria and suitability to be used as inoculum for continuous fermentation as shown in FIG. 3, showing the initial stabilization phase of a bioreactor inoculated with 1% of cryopreserved effluent after 7 days. The fermentation reached a metabolic profile comparable to the continuous fermentation used as effluent for cryopreservation. The metabolite concentrations of the last days of the latter are plotted on day −3 to −1.

Viability if over 60% is typically observed after stabilization. Lower viability is observed in preserved inocula after storage, e.g. a survival of as low as 5% or 10% has been observed in preserved samples of an in vitro assembled consortium after eight months of storage. Nevertheless, such preserved samples when used as an inoculum in the method of manufacture according to the present invention still resulted in the manufacture of the same in vitro assembled consortium with the characteristic microbial profile and metabolic profile of the preserved consortium.

### Example 6: Inoculum for Large-Scale Production

In order to produce a defined consortium at industrial scale, e.d. more than 50 L, the fermentation process needs to guarantee reproducible production within the defined specifications.

Since all biotechnological processes start with a defined inoculum, both preservation methods described in example 6 were applied to the single strains contained in the exemplary consortium PB002 and the consortium PB002 produced in continuous co-culture as described in example 2 and compared for their suitability as inoculum for continuous co-cultivation of in vitro assembled consortia. The previously established continuous fermentation inoculated with fresh single cultures as described in example 2 was used as control.

FIG. 4 shows the metabolic profile of continuous fermentations inoculated with 1% of:

(1) Control reactor inoculated with mix of independently cultured fresh cultures of the 9 strains in PB002 (prepared in two steps as described above in example 2);

(2) Bioreactor inoculated with cryopreserved PB002, stored for 3 months at −20° C. in a cryoprotective glycerol solution (prepared as described in example 5);

(3) Bioreactor inoculated mix of the 9 single strains contained in PB002 stored independently for 3 months in the same glycerol solution and mixed after thawing;

(4) Bioreactor inoculated lyophilised PB002 stored for 6 months at 4° C. and resuspended in the dispersing medium (prepared as described in example 5);

(5) Bioreactor inoculated with a mix of 6-month-old independently lyophilised cultures of the 9 strains in PB002.

The cryopreserved PB002 inoculum and the lyophilised PB002 inoculum prior to their preservation comprised the stable PB002 consortium after co-cultivation as described in example 2. Metabolic profiles were compared after 7 days of stabilization and showed that both preservation methods show a production of the desired metabolites, acetate, propionate and butyrate in the expected ratios with equal concentrations of propionate and butyrate both more than 10 mM, and more than 20 mM of acetate. The bacteria that were produced separately and mixed after storage, did not grow to the desired ratios and respective metabolic profiles, showing a strong reduction of butyrate and propionate production if used as inoculum for the continuous fermentation process described above. The qPCR analysis (as described in example 4) of the single strains and their abundance in the bioreactors at day 7 after inoculation (FIG. 5) showed the maintenance of all strains in groups (1), (2) and (4). Strains were at the desired levels, comparable to the continuous fermentation process using a non-preserved strain mix (1), in the groups (2) and (4) that were inoculated with a preserved inoculum produced in mixed culture and cryopreserved in the first case and lyophilised in the latter. Independent cultivation previous to preservation resulted in drastic reduction and even loss of single strains in the consortium (2) and (5).

These data showed that cryopreservation and lyophilisation of a stable consortium supports the maintenance of metabolic and compositional profile of intestinal consortia during the preservation, storage, and reactivation. The used of an inoculum produced in mixed culture results in a re-establishment of the metabolic and bacterial profile characteristic of the stored consortium during subsequent anaerobic co-cultivation while the use of separately cryopreserved bacteria result in variable survival and is thus not appropriate for production of bacterial consortia.

### Example 7: Transferability of Method for the Establishment of In Vitro Assembled Consortia

Dependent of the targeted combination of functional groups, the approach presented in example 2 can be used for a multitude of in vitro assembled consortia resulting of combinations of the functional groups (A1)-(A9) or of (A1)-(A10), if the choice of functional groups is based on metabolic interactions that mutually stabilize the levels of intermediate concentrations and thereby also the levels of abundance of each of the selected bacterial strains in anaerobic co-cultivation, in particular by fulfilling criteria (a) and (b). In FIG. 6, an in vitro assembled consortium including the functional groups from (A1)-(A7) and (A9)-(A10) was assembled (PB003). Using the method described in example 2, the bacterial consortium stabilized after 7 days and produced the expected metabolites acetate and butyrate, while the lack of the group (A8) resulted in a non-inhibiting accumulation of succinate and a reduced production of propionate as compared to PB002. Therefore, the method to assemble consortia can be used according to the claim 1.

### Example 8: Reproducibility of Process in Continuous Fermentation

In order to validate the suggested process for industrial production the therapeutic/exemplary consortium PB002 was produced in three independent batches using 1% of the cryopreserved inoculum (FIG. 7, 1-3) and 1% of the lyophilised inoculum (FIG. 7, 4-6), respectively. Using the process described in example 3, all repetitions stabilized at the targeted metabolite concentrations and relative abundances dominated by acetate in combination with at least 20% of butyrate and propionate each after 7 days of continuous fermentation using the process described in example 2. The suggested stabilization methods are therefore reproducible methods for the production of microbial consortia.

### Example 9: Production of In Vitro Assembled Consortia Using Batch Fermentation

The exemplary consortium PB002 was lyophilised as described in example 5 and used as inoculum for a batch fermentation.

FIG. 8 shows the mean bacterial metabolite concentration in three different bioreactors. The bioreactors were inoculated with 1% lyophilised inoculum as described in example 10 after 48 h of batch fermentation (1) to (3). Used inocula were produced using the continuous co-cultivation method described in example 2 and stored for at least 3-month at 4° C. All three independent fermentations showed of all desired metabolites, acetate, propionate and butyrate in comparable ratio proving reproducibility.

For the first time it has been shown that an in vitro assembled consortium of selected bacterial strains can be produced by multiplying an inoculum of the consortium in an anaerobic batch cultivation and harvesting the same consortium of bacterial strains as used for inoculation as product. The resulting very high reproducibility of the microbial and metabolic profile is characteristic for the consortium. This reproducibility is even enhanced if the sample used as inoculum after assembling the selected strains from single cultures is produced in an anaerobic co-cultivation, in particular, if followed by a post-treatment of preservation by cryopreservation or lyophilization.

### Example 10: Single Strains do not Grow on Fermentation Medium Alone

In order to show that co-culture is superior to single culture, the growth and metabolic activity of all single strains contained in the consortium PB002 was compared to co-cultivated PB002 using a batch fermentation on the medium used for co-cultivation in continuous fermentation.

FIG. 9 shows the growth of the single bacteria of the exemplary consortium PB002 inoculated in Hungate tubes in triplicates with 0.8 mL of a 1:10 dilution after 48 h of culture in 3-times buffered fermentation medium as specified in example 14 as compared to the inoculation of 0.8 mL of a 1:10 dilution of effluent from a continuously operated bioreactor containing PB002 (day 15 of fermentation) inoculated to the same medium.

Optical density (OD600) was measured after 48 h of cultivation and completed with strain-specific qPCR quantification as described above.

Both quantification methods showed an impaired growth of single strains as compared to the same strains in co-cultivation when cultivated on the same medium.

After 48 h of batch fermentation only strain 4 representing the functional group A4 was able to grow to an optical density (OD600) comparable to the OD600 observed in co-cultivation indicating their limited capacity of all other strains to grow in a simplified medium if not co-cultivated with the defined functions to control and support their growth.

qPCR quantification of the single strains confirms absence of growth of the strains 1, 2, 5 and 8 representing the functional groups A1, A2, A5 and A8, whereby A1 and A2 were not capable to use the available substrate in isolation while A8 and A5 were missing their respective substrate since they rely on the production of intermediate metabolites produced by another strain.

In conclusion, the co-cultured strains of PB002 showed superiority compared to single cultures in their capacity to grow on simplified media as opposed to the highly complex media used for strict anaerob cultivation.

### Example 11: Experimental Validation of Cross Feeding for Consortium Design

In order to validate the interactions of single stains from the functional groups described in FIG. 1 in vivo, pairs of two strains connected through a metabolite were co-cultivated on YCFA medium containing starch as carbon source. using Hungate tube technique.

0.3 mL of each 48 h culture of the single strains were inoculated alone or in pairs after standardization to an OD600 of 1.

Each strain was inoculated in triplicate for each condition. Single cultures were compared to the co-cultivation of the relative pairs at 24 h and 48 h of growth.

Pairs were chosen according to FIG. 1, combining a starch-degrading primary degrader and a corresponding reutiliser of the produced metabolites (intermediate metabolites).

The following combinations are represented in FIG. 10 as a representative selection of possible combinations:
B. adolescentis (A4) and E. limosum (A6/A9) (lactate/formate-producer and lactate/formate-consumer/butyrate-producer) (1);
Lb. rhamnosus (A3) and A. lactatifermentans (A5) (lactate-producer and lactate-consumer/propionate producer) (2); and
B. xylanisolvens (A10) and P. faecium (A8) (succinate-producer and succinate-consumer/propionate producer) (3)

Optical densities measured after 24 and 48 h showed an improved growth of the co-cultivated pairs as compared to the isolated cultivation of the single strains confirming the beneficial effect of cross-feeding on growth of the single strains, by allowing an increased extraction of energy from the medium.

The cross-feeding was confirmed in the metabolic profiles of the single condition as compared to the co-cultivated conditions.

The first condition described in column 1 of FIG. 10 shows production of acetate, formate and lactate by the B. adolescentis (A4) in single culture while in co-culture with E. limosum (A6) that produces acetate and butyrate when cultivated alone, we measured an increased total growth as measured by the OD600 in row A column 1 and a reduction of the presence of formate and a depletion of lactate in the medium while increasing butyrate production as shown in row B of the column 1. Thereby confirming the predicted cross-feeding of the functional groups A4 and A6 in vitro.

The column 2 shows cocultivation of Lb. rhamnosus (A3) that showed Lactate and formate production in single culture with A. lactatifermentans (A5) a known lactate utilizer showed a decrease of lactate and increase of propionate in co-cultivation as compared to the single culture of A. lactatifermentans (column 2, row B). The OD600 of the co-cultivated strains being higher than the OD of the single strains (column 1, row A), we confirmed the utilisation of lactate for the production of propionate as predicted and a subsequent increase of total biomass produced.

In a third condition shown in the column three B. xylanisolvens (A10) produces lactate, formate and succinate that was subsequently used by P. faecium (A8) a propionate producing succinate utilizer.

Besides the increased growth, seen in column 3 row A we see an increase of propionate as compared to the single culture condition of P. faecium and a depletion of the succinate produced as observed in the single culture of B. xylanisolvens in column 3 row B.

In conclusion, for all co-cultivated bacterial mixes, an increased optical density could be measured after 24 h and 48 h of co-cultivation as compared to the single cultures.

For co-culture 1, complete lactate-utilization was seen after 24 h of fermentation as well as a decrease in formate concentration from 24 h to 48 h confirming the predicted cross-feeding.

For co-culture 2 decrease in lactate concentration combined with a clear increase of propionate was seen from 24 h to 48 h indicating a metabolic succession.

For co-culture 3, complete utilization of the succinate produced by B. xylanisolvens was seen after 24 h of fermentation resulting propionate production.

### Example 12: Comparison of Inoculum Production Methods for Batch Production of Consortia

To establish a method for the production of consortia based on cross-feeding and in a physiologically relevant ratio, as confirmed using continuous fermentation, we explored three types of inoculum production and two types of conservation, namely cryo-preservation and lyophilisation using our exemplary consortium PB002 as listed in the table 4 below:

| Inoculum production | Product production (A) | Product production (B) |
|---|---|---|
| Step 1 | Step 2A | Step 2B |
| Batch + | Cryopreserved Inoculum | Lyophilized Inoculum |
| Continuous (1) | | |
| Batch (2) | Cryopreserved Inoculum | Lyophilized Inoculum |
| Continuous (3) | Cryopreserved Inoculum | Lyophilized Inoculum |

In order to show the necessity of inoculum production process using batch fermentation with subsequent continuous fermentation, we produced PB002 inocula in 3 different ways:
through the whole disclosed inoculum production process using batch and continuous fermentation (Batch+Continuous (1)),
through the first part of the inoculum production process using only batch fermentation (Batch (2)),
through the second part of the inoculum production process using only continuous fermentation (Continuous (3)).

At the end of the 3 fermentations effluent was stored in two different ways:
Cryopreserved using a medium containing glycerol as specified in example 5
Lyophilized using the lyophilization buffer as specified in example 5

These 6 differently produced inocula were used to inoculate step 2 of the disclosed process, 48 h batch fermentations that lead to the final product. Thereby, step 2A was initiated with the cryopreserved inocula and step 2B with the 3 different lyophilized inocula.

After 48 h of the batch fermentations we compared the microbial profiles of the 6 different products. FIG. 11 shows the absolute difference in abundance of each strain of the consortium as compared to the desired composition that is defined by the composition of the consortium strains when cultivated under gut-like continuous fermentation conditions as described in example 14. The desired composition represents the relative abundance of co-cultured strains at the point of inoculum preservation (end of step 1, using batch and subsequent continuous fermentation).

The difference in relative abundance to the desired composition were quantified using specific qPCR primers as described in example 4 and are indicated in copies of the 16S rRNA gene/ml of culture for the strains representing A1 to A9. Error bars represent standard deviations of 3 technical replicates. Two-way ANOVA was performed. Significance (*) is defined with a p-value <0.05.

The data demonstrate that the desired microbial profile established using a continuously produced inoculum can only be reproduced in batch fermentation initiated with inoculum “Batch+Continuous (1)”.

Especially the more sensitive strains are disadvantaged when the inoculum was produced through parts of the original inoculum production process only (2, and 3), such as R. bromii, F. prausnitzii and B. hydrogenotrophica. The effect is even more pronounced for the lyophilized inocula (B).

### Example 13: Presence of all Strains of the Consortium

In order to validate the presence of all strains necessary to maintain stability of a consortium, presence of all strains in a continuously operated bioreactor was measured over 12 weeks of continuous operation as described in example 2. For the exemplary consortium PB002 composition was quantified using specific qPCR primers for all 9 members of the consortium qPCR quantification of the single strains of the consortium was performed using the primers listed in table 5.

TABLE 5

| Group *) | Bacteria strains | Primer FW 5′-3′ | Primer RV 5′-3′ |
|---|---|---|---|
| A1 ¹⁾ | Ruminococcus | CGCGT GAAGG ATGAA | TCAGT TAAAG CCCAG |
| | bromii | GGTTT TC | CAGGC |
| A2 ¹⁾ | Faecalibacterium | CGCGG TAAAA CGTAG | CTGGG ACGTT GTTTC |
| | prausnitzii | GTCAC A | TGAGT TT |
| A3 ¹⁾ | Lactobacillus | GGAAT CTTCC ACAAT | CATGG AGTTC CACTG |
| | rhamnosus | GGACG CA | TCCTC TT |
| A4 ¹⁾ | Bifidobacterium | GTCCATCG CTTAACGG | ACCAC CTGTG AACCC |
| | adolescentis | TGGATC | GC |
| A5 ¹⁾ | Clostridium | GCACT CCACC TGGGG | CAACC TTCCT CCGGG |
| | (Anaerotignum) | AGT | TTATC CA |
| | lactatifermentans | | |
| A6 ²⁾ | Eubacterium | GGCTT GCTGG ACAAA | CTAGG CTCGT CAGAA |
| | limosum | TACTG | GGATG |
| A7 ¹⁾ | Collinsella | GGTAG GGGAG GGTGG | GCGGT CCCGC GTGGG |
| | aerofaciens | AAC | TT |
| A8 ¹⁾ | Phascolarcto- | GGAGT GCTAA TACCG | CCGTG GCTTC CTCGT |
| | bacterium faecium | GATGT GA | TTACT |
| A9 ¹⁾ | Blautia | CGTGA AGGAA GAAGT | TCAGT TACCG TCCAG |
| | hydrogenotrophica | ATCTC GGTA | CAGGC C |
| A1-A9 ³⁾ | All bacteria | GTGST GCAYG GYTGT | ACGTC RTCCC CRCCT |
| | | CGTCA | TCCTC |

*) sources: ¹⁾DECIPHER database; ²⁾Wang et al. (1996), ³⁾Maeda et al., (2003)

DNA from pellets of the fermentation effluent was extracted using the FastDNA™ SPIN Kit for Soil (MP Bio). Genomic DNA extracts were 50-fold diluted using DNA-free H₂. qPCRs were performed using Mastermix SYBR® green 2× and LowRox (Kapa Biosystems), primers (10 μM) and DNA-free H₂O were used in a ABI 7500 FAST thermal cycler (Applied Biosystems) as recommended bythe producer and quantified using standards of amplified whole 16S rRNA gene amplicon sequences of the strains used for the consortium cloned into the pGEMT easy vector (Promega, Madison Wis., USA). Amplification of the whole 16S rRNA gene was performed with a combination of whole 16S rRNA gene amplification primers using one forward and one reverse primer of the primers listed in Table 5. qPCR quantification of the single strains is shown in log 10 copies of genomic 16S rRNA gene per ml of culture in FIG. 12 showing the maintenance of all 9 strains in our model consortium over 12 weeks of continuous operation of the bioreactor.

### Example 14: Assembly of Alternative Consortium Containing Functional Groups A1-A9 with Other Strains of the Same Species as PB002

Composition PB004

| Bacterial strain | Reference | Functional group |
|---|---|---|
| R. bromii | ATCC 27255 | A1 |
| F. prousnitzii | DSM 17677 | A2 |
| Lb. rhomnosus | DSM 20021 | A3 |
| B. adolescentis | DSM 20083 | A4 |
| A. lactatifermantans | DSM 14214 | A5 |
| E. limosum | DSM 20543 | A6 |
| C. aerofaciens | DSM 3979 | A7 |
| P. faecium | DSM 14760 | A8 |
| B. hydrogenotrophica | DSM 10507 | A9 |

In order to establish an alternative consortium (PB004 as described above) using the same rules of assembly and species as used for PB002 in a growing and metabolically interacting manner, a previously validated medium for PB002 was adapted using a simplified medium based on YCFA (DSMZ Media N° 1611). Thereby, the 5 g/L of glucose that are the carbon source in YCFA were replaced by 3 g/L of cellobiose (Sigma Aldrich), 2 g/L of fructo-oligosacharaides (FB97, Cosucra), 3 g/L of soluble potato starch (Sigma Aldrich), and 4 g/L of pea starch (Roquette). A 500 ml bioreactor (Infors HT) was inoculated with a mix of overnight cultures of all 10 strains and inoculated anaerobically at a 1/100 dilution. The bioreactor was consecutively operated at pH 6.0 for 24 h in order to allow growth of primary degraders and subsequent consumption of the produced intermediate metabolites. Growth was monitored by base consumption and optical density. Metabolites were monitored using HPLC-RI as described above. After the first batch-fermentation, new medium was fed by removing half of total volume and refilling with medium to the original volume of 500 ml in the bioreactor. After the second batch fermentation cycle the metabolic profile did not contain any intermediate metabolites and >40 mM acetate and >5 mM of propionate and butyrate each (FIG. 13). From the end of the second batch fermentation on, the bioreactor was operated continuously at a volume of 500 ml, a flow rate of 10.0 ml/h and a pH of 6.0. Subsequently, a stable metabolic profile established within 7 days after inoculation containing exclusively the desired end metabolites of acetate, propionate and butyrate without detection of intermediate metabolites showing constant production of all desired metabolites without washout of any functional group.

PB004 could therefore be cultured in a bioreactor and showed the desired properties of the intestinal microbiome, i.e. degradation of fibers and proteins into exclusively end-metabolites, a clear indication that the desired interactions and metabolic activities described in example 13 were established in a continuously operated bioreactor

### Example 15: Assembly of Alternative Consortium Combining Two Functional Groups (A6 and A9) with One Bacterium

In order to establish a consortium that harbours the same functions as PB002 but with fewer bacteria, a consortium containing a bacterium capable of covering two functional groups (A6 and A9) was developed. In this case, E. limosum was used to combine the functional groups A6 and A9. PB010 was assembled using the same rules as used for PB002 in a growing and metabolically interacting manner, a previously validated for PB002 was adapted using a simplified medium based on YCFA (DSMZ Media N^{o} 1611).

Composition PB010:

| Bacterial strain | Functional group |
|---|---|
| R. bromii | A1 |
| F. prousnitzii | A2 |
| Lb. rhomnosus | A3 |
| B. adolescentis | A4 |
| A. lactablermentans | A5 |
| E. limosum | A6 + A9 |
| C. aerofaciens | A7 |
| P. faecium | A8 |

Thereby, the 5 g/L of glucose that are the carbon source in YCFA were replaced by 3 g/L of cellobiose (Sigma Aldrich), 2 g/L of fructo-oligosacharaides (FB97, Cosucra), 3 g/L of soluble potato starch (Sigma Aldrich), and 4 g/L of pea starch (Roquette). Thereby, the 5 g/L of glucose that are the carbon source in YCFA were replaced by 2 g/L of pectin (Sigma Aldrich), 1 g/L of fructo-oligosacharaides (FB97, Cosucra), 3 g/L of potato starch (Sigma Aldrich), and 2 g/L of corn starch (Sigma Aldrich). A 500 ml bioreactor (Infors HT) was inoculated with a mix of overnight cultures of all 10 strains and inoculated anaerobically at a 1/100 dilution. The bioreactor was consecutively operated at pH 6.0 for 24 h in order to allow growth of primary degraders and subsequent consumption of the produced intermediate metabolites. Growth was monitored by base consumption and optical density. Metabolites were monitored using HPLC-RI as described above. After the first batch-fermentation, new medium was fed by removing half of total volume and refilling with medium to the original volume of 500 ml in the bioreactor. After the second batch fermentation cycle the metabolic profile did not contain any intermediate metabolites and >40 mM acetate and >5 mM of propionate and butyrate each (FIG. 14). From the end of the second batch fermentation on, the bioreactor was operated continuously at a volume of 500 ml, a flow rate of 10.0 ml/h and a pH of 6.0. Subsequently, a stable metabolic profile established within 7 days after inoculation containing exclusively the desired end metabolites of acetate, propionate and butyrate without detection of intermediate metabolites showing constant production of all desired metabolites without washout of any functional group.

PB010 could therefore be cultured in a bioreactor and showed the desired properties of an intestinal microbiome, i.e. degradation of fibers and proteins into exclusively end-metabolites, a clear indication that the desired interactions and metabolic activities described in example 13 were established in a continuously operated bioreactor. It also showed that the selected strain of E. limosum was capable of combining the two functional groups A6 and A9 into one bacterium as seen be the presence of exclusively end-metabolites.

### Example 16: Assembly of Consortium Containing Functional Groups A1-A10

In order to establish an alternative consortium using the same rules of assembly as for PB002 in a growing and metabolically interacting manner, a previously validated for PB002 was adapted using a simplified medium based on YCFA (DSMZ Media N^{o} 1611).

Composition: PB011

| Bacterial strain | Functional Group |
|---|---|
| Eubacterium eligens | A1 |
| Roseburia intestinalis | A2 |
| Enterococcus faecalis | A3 |
| Roseburia hominis | A4/A7 |
| Coprococcus catus | A5 |
| Eubacterium hallii | A6 |
| Eubacterium limosum | A6/A9 |
| Flavomfractor plautii | A8 |
| Bacteroides xylanisolvens | A10/A11 |

Thereby, the 5 g/L of glucose that are the carbon source in YCFA were replaced by 3 g/L of cellobiose (Sigma Aldrich), 2 g/L of fructo-oligosacharaides (FB97, Cosucra), 3 g/L of soluble potato starch (Sigma Aldrich), and 4 g/L of pea starch (Roquette). A 500 ml bioreactor (Infors HT) was inoculated with a mix of overnight cultures of all 10 strains and inoculated anaerobically at a 1/100 dilution. The bioreactor was consecutively operated at pH 6.0 for 24 h in order to allow growth of primary degraders and subsequent consumption of the produced intermediate metabolites. Growth was monitored by base consumption and optical density. Metabolites were monitored using HPLC-RI as described above. After the first batch-fermentation, new medium was fed by removing half of total volume and refilling with medium to the original volume of 500 ml in the bioreactor. After the second batch fermentation cycle the metabolic profile did not contain any intermediate metabolites and >40 mM acetate and >5 mM of propionate and butyrate each (FIG. 15). From the end of the second batch fermentation on, the bioreactor was operated continuously at a volume of 500 ml, a flow rate of 10.0 ml/h and a pH of 6.0. Subsequently, a stable metabolic profile established within 7 days after inoculation containing exclusively the desired end metabolites of acetate, propionate and butyrate without detection of intermediate metabolites showing constant production of all desired metabolites without washout of any functional group.

PB011 could therefore be cultured in a bioreactor and showed the desired properties of the intestinal microbiome, i.e. degradation of fibers and proteins into exclusively end-metabolites, a clear indication that the desired interactions and metabolic activities described in example 12 were established in a continuously operated bioreactor.

### REFERENCES

Throughout this specification, the following references are cited:
Bryant, M. P. (1972). Commentary on the Hungate technique for culture of anaerobic bacteria. The American Journal of Clinical Nutrition, 25(12), 1324-1328.
Chassard, C. & Lacroix, C., 2013. Carbohydrates and the human gut microbiota. Current opinion in clinical nutrition and metabolic care, 16(4), 453-460.
Duncan, S. H., Hold, G. L., Harmsen, H. J. M., Stewart, C. S., & Flint, H. J. (2002). Growth requirements and fermentation products of Fusobacterium prausnitzii, and a proposal to reclassify it as Faecalibacterium prausnitzii gen. nov., comb. nov. International Journal of Systematic and Evolutionary Microbiology, 52(6), 2141-2146.
Kaufmann, P., Pfefferkorn, A., Teuber, M., & Meile, L. (1997). Identification and quantification of Bifidobacterium species isolated from food with genus-specific 16S rRNA gene-targeted probes by colony hybridization and PCR. Applied and Environmental Microbiology, 63(4), 1268-1273.
Khan, M. T., Duncan, S. H., Stams, A. J. M., van Dijl, J. M., Flint, H. J., & Harmsen, H. J. M. (2012). The gut anaerobe Faecalibacterium prausnitzii uses an extracellular electron shuttle to grow at oxic-anoxic interphases. The ISME Journal, 6(8), 1578-1585.
Lane, D. J. (1991). 16S/23S rRNA Sequencing. In E. Stackebrandt & M. Goodfellow (Eds.), Nucleic Acid Techniques in Bacterial Systematics (Chapter 6 pp. 115-148). Sussex: John Wiley and Sons.
Leclerc, M., Bernalier, A., Donadille, G., & Lelait, M. (1997). H2/CO2 metabolism in acetogenic bacteria isolated from the human colon. Anaerobe, 3(5), 307-315.
Maeda, H., Fujimoto, C., Haruki, Y., Maeda, T., Kokeguchi, S., Petelin, M., Takashiba, S. (2003). Quantitative real-time PCR using TaqMan and SYBR Green for Actinobacillus actinomycetemcomitans, Porphyromonas gingivalis, Prevotella intermedia, tetQ gene and total bacteria. FEMS Immunology and Medical Microbiology, 39(1), 81-86.
Mosoni, P., Chaucheyras-Durand, F., Bera-Maillet, C., & Forano, E. (2007). Quantification by real-time PCR of cellulolytic bacteria in the rumen of sheep after supplementation of a forage diet with readily fermentable carbohydrates: effect of a yeast additive. Journal of Applied Microbiology, 103(6), 2676-85.
Muyzer, G., de Waal, E. C., & Uitterlinden, A. G. (1993). Profiling of complex microbial populations by denaturing gradient gel electrophoresis analysis of polymerase chain reaction-amplified genes coding for 16S rRNA. Applied and Environmental Microbiology, 59(3), 695-700.
Wang, R. F., Cao, W. W., & Cerniglia, C. E. (1996). PCR detection and quantitation of predominant anaerobic bacteria in human and animal fecal samples. Applied and Environmental Microbiology, 62(4), 1242-1247.
Zihler, A., Fuentes, S., Dostal, A., Payne, A. N., Vazquez Gutierrez, P., Chassard, C., Grattepanche F., de Vos W. M., Lacroix, C. (2013). Novel Polyfermentor Intestinal Model (PolyFermS) for Controlled Ecological Studies: Validation and Effect of pH. PloS One, 8(10).

1-47. (canceled)

48. A method of manufacturing an in vitro assembled consortium of selected live, viable bacterial strains by an anaerobic co-cultivation in a dispersing medium,
wherein the consortium comprises a plurality of functional groups, each group comprising at least one of the selected bacterial strains,
wherein each functional group of selected bacterial strains performs at least one metabolic pathway of an anaerobic microbiome, in particular of an intestinal microbiome,
wherein the method of manufacturing comprises the steps of
I. providing a sample of the assembled consortium as an inoculum,
wherein the sample of the consortium is obtained from a prior continuous anaerobic co-cultivation process of the selected bacterial strains until a stable microbial profile and a stable metabolic profile characteristic of the in vitro assembled consortium has been established, and
wherein the sample is obtained as a preserved sample;
II. adding the inoculum to the dispersing medium in a bioreactor thereby forming a culture-suspension of the selected bacterial strains;
III. multiplying the selected bacterial strains in the culture suspension by co-cultivation until a stable microbial profile and a stable metabolic profile characteristic of the in vitro assembled consortium is established;
IV. harvesting the consortium of the selected live, viable bacterial strains;
V. optionally, subjecting the harvested consortium to one or more post-treatment steps;
characterized in that step III is performed in an anaerobic batch fermentation process or in an anaerobic fed-batch fermentation process.
